(19) United States
(12) Patent Application Publication
(10) Pub. No.: US 2009/0012088 A1
(43) Pub. Date: Jan. 8, 2009

(54) Compounds for the Treatment of Neurodegeneration and Stroke

(76) Inventors: Paul J. HERGENROTHER, Champaign, IL (US); Karson S. PUTT, Champaign, IL (US); Xianjun LIU, Urbana, IL (US)

Correspondence Address: GREENLEE WINNER AND SULLIVAN P C, 4875 PEARL EAST CIRCLE, SUITE 200, BOULDER, CO, 80301, US

(21) Appl. No.: 12/129,047

(22) Filed: May 29, 2008

Related U.S. Application Data
(62) Division of application No. 11/460,073, filed on Jul. 26, 2006, now abandoned.
(60) Provisional application No. 60/702,445, filed on Jul. 26, 2005.

(51) Int. Cl. A61K 31/5377; A61K 31/50; A61P 25/00; A61K 31/501

(52) U.S. Cl. 514/236.5; 514/247; 514/252.05; 514/252.03

(57) ABSTRACT

Compounds and related methods for synthesis, and the use of compounds for the treatment of neurodegenerative diseases are disclosed. Compounds are disclosed in connection with PARG and/or PARP inhibition. Therapeutic applications are relevant for preventing or inhibiting neurological cell death for a variety of neurodegenerative conditions including Parkinson's disease, ischemia, and stroke. Also disclosed is a high-throughput screen for identifying compounds capable of inhibiting PARG and/or PARP.

### CROSS REFERENCE TO RELATED APPLICATIONS

This application is a divisional application of U.S. patent application Ser. No. 11/460,073 filed Jul. 26, 2006 which claims the benefit of U.S. Provisional Application No. 60/702,445, filed Jul. 26, 2005, each of which are incorporated herein by reference in their entirety to the extent not inconsistent herewith.

### BACKGROUND OF THE INVENTION

DNA repair pathways have been targeted for manipulation by medical therapeutics because DNA repair is important for regulation of cell growth and survival. The enzymes poly(ADP-ribose) glycohydrolase (PARG) and poly(ADP-ribose) polymerase (PARP) are associated with DNA repair. Accordingly, there is a great deal of interest in developing compounds to inhibit PARP and/or PARG. For example, PARG is involved in neuronal cell death (Putt and Hergenrother, 2004). Neurodegenerative disorders, including Parkinson's, Alzheimer's and ALS, are an enormous medical problem that currently afflicts 10-20% of people over the age of 65. At present there are few treatments and no cures for these diseases. Therefore, PARG inhibitors may be useful as medicaments for alleviating and/or treating neurological disorders, including Parkinson's disease.

PARP and PARG are both associated with DNA repair, apoptosis and necrosis. When DNA is mildly damaged, PARPs utilize NAD⁺ to create poly(ADP-ribose) (PAR) on the glutamic acid residues of proteins (including on PARP-1 itself), altering the overall charge and size of the modified protein, and ultimately initiating DNA repair.¹ PAR biopolymers consist of up to about 200 ADP-ribose units, and polymer levels may increase more than 100-fold in minutes.² This poly(ADP-ribosyl)ation of proteins is transient, as PARG operates in both an endo- and exoglycosidic fashion to break down PAR into ADP-ribose monomers.³ PARG catalyzes the hydrolysis of glycosidic bonds of ADP-ribose polymers to produce monomeric ADP-ribose units. PARP-1 also catalyzes the formation of PAR polymers to itself. The polymerized PAR-PARP-1 complex releases the DNA due to electrostatic repulsion with the damaged DNA and the DNA damage is then repaired. The PAR-PARP-1 complex remains in an inactive state, unable to prime damaged DNA for repair, until PARG degrades the PAR polymer. Once the PAR polymer is degraded, PARP-1 can once again bind damaged DNA. Therefore, PARG is required for continued function of PARP-1 DNA repair. In addition to its role in DNA repair and replication, PAR is also involved in control of chromosome migration during cell division, activation of transcription after DNA damage, activation of the proteasome, regulation of telomere maintenance, spindle assemble and function. PAR metabolism is a necessary part of normal neuronal function.

If DNA damage is relatively more severe, caspases inactivate PARP-1 by cleavage, thereby preventing DNA repair via PARP, and the cell undergoes apoptosis. For extremely severe DNA damage, PARP-1 is overactivated, resulting in excessive NAD⁺ depletion and associated breakdown of glycolysis and the Krebs cycle, leading to necrosis.

Because PARP is involved in DNA repair, extended PARP inhibition can have adverse side effects, including increased mutation rates with associated increase in the incidence of cancer. Accordingly, inhibition of PARG, instead of inhibition of PARP, can minimize obstruction of DNA repair mechanisms. In addition, inhibiting PARP prevents formation of PAR, and thus disrupts PAR's normal metabolic function. However, PARG inhibition instead locks PARP into its fully automodified state (the PAR-PARP-1 complex), which is inactive. Therefore, PARG inhibition permits PAR to maintain its normal function within the cell, thereby avoiding or minimizing adverse side-effects that are often associated with application of PARP inhibitors.

PARG inhibitors have been shown to reduce ischemic injury, suppress tumor virus gene expression, and prevent oxidative neuronal cell death. In addition, PARG is involved in apoptosis because it is a substrate for caspase-3³⁶.

PARG inhibitors are important for further elucidating PARG function in vivo, as well as for medical therapeutics. A recently developed convenient and high-throughput assay permits screening of potential PARG inhibitors (Putt and Hergenrother (2004), hereby incorporated by reference in its entirety, and Putt et al. (2005), hereby incorporated by reference in its entirety and attached herein as Appendix A).

Although there are a myriad of PARP-1 inhibitors, very few inhibitors of PARG have been described. Currently, PARG inhibitors can be classified into three major classes: ADP-ribose analogues, DNA intercalators, and tannins. Tannin molecules, however, are only modestly potent inhibitors and are high molecular-weight species making delivery to cells difficult. The other two classes are also modestly effective PARG inhibitors, but also suffer drawbacks. For example, DNA intercalators suffer from considerable toxicity. ADP-ribose analogues lack cell permeability, and because they closely resemble biologics they may have additional side effects. Accordingly, there is a need in the art for novel small-molecule inhibitors of PARG.

Herein we disclose small molecule inhibitors of PARG. Furthermore, we demonstrate the ability of these small molecules to function as neuroprotective agents for whole cells challenged with DNA damaging agents. We believe the small molecules disclosed herein are the first effective cell-permeable small molecule inhibitors of PARG. The small molecules and related methods disclosed herein represent a novel and valuable approach for treating diseases, including stroke and other neurologically-damaging disorders, and certain cancers, including breast cancers with the BRCA2 mutation.

### SUMMARY OF THE INVENTION

The invention broadly provides compounds, methods of making compounds, methods of therapeutic treatment, methods of screening for compounds, and methods of screening for cell and patient suitability for treatment in connection with inhibitors of PARG. In embodiments, the inventions are applicable in the context of a variety of neurological diseases and disorders such as Parkinson's disease, as well as neurological complications arising from adverse biological events, including ischemia and stroke. In an aspect, the compounds are small-molecule inhibitors of PARG and can be used to treat a variety of biological conditions, including but not limited to, Parkison's disease. Because of the different function played by PARG during different DNA damaging events (e.g. DNA repair for mild damage, apoptosis for intermediate damage, and necrosis for severe damage), the PARG inhibitors of the present invention are also amenable for treatment of other diseases including certain cancers, particularly breast cancers, and more particularly breast cancers with the BRCA2 mutation.

An embodiment of the invention is a compound of formula FX1:

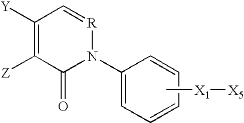

Wherein R is C or N; Y is NH₂, NR₁R₂, halide or acyl; R₁ and/or R₂ are (independently of each other) alkyl groups ranging from between one and about ten carbons inclusive; alkyl groups where two alkyl groups form a cyclic moiety having 5, 6, 7 or 8 ring members where one ring carbon can be O, hydrogen, aryl, substituted alkyl where one or more substituents are halides, alkyl aryl;

wherein Z is NH₂, NR₄R₅, halide, a group that is converted under physiological conditions to NH₂; R₄ and/or R₅ can have values (independently of each other) of R₁, and/or R₂, COR₆, where R₆ is H or small alkyl; and

wherein X₁-X₅ is (independently of each other) hydrogen, halide, alkyl, alkylhalide, OH, OCH₃, OR₇, methyltrihalide, CF₃, where R₇ is alkyl Specifically, X₁ which is meta or para to the C—N bond can have all values given for X₁-X₅.

In an embodiment, the compound has structure FX2:

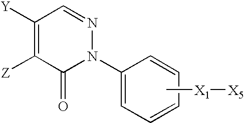

or structure FX3:

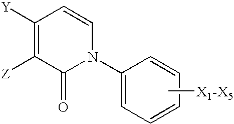

Wherein Y, Z and X₁-X₅ are defined as above.

In an embodiment, the compound has structure FX4:

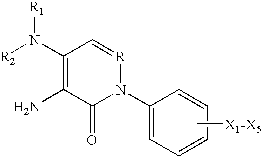

Wherein R, R₁, R₂ and X₁-X₅ are as defined for FX1.

In an embodiment, the compound is any of structures FX1-4, with the exception of PIP-1:

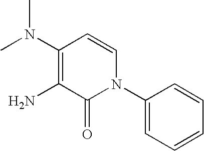

The compound, in an embodiment, is selected from the group consisting of structures 1-39 as provided in Table 2. In an embodiment, the compound is selected from the group consisting of structures providing good and moderate activity as provided in Table 2 (e.g. structures 1, 11-15, 17-25, 37). In an embodiment, the compound is selected from the group consisting of structures 1-22, and 24-39. In an embodiment, the compound is selected from the group consisting of structures 1, 11-13, 15, 17-22, 24, and 25, 37. In an embodiment, the compound is selected from the group consisting of structures 11-12, 14, and 19-21, 37. In an embodiment, the compound is selected from the group consisting of structures 12, 13, 19 and 37, or from the group consisting of structures 12, 13 and 19.

In an embodiment, a composition of the invention is a neuroprotective agent. In an embodiment, a composition of the invention is a chemotherapeutic agent. In an embodiment the compound of the present invention is capable of inhibiting PARG. In an embodiment the compound of the present invention is capable of inhibiting PARP. In another aspect, the compound has an activity level categorized as moderate to good, wherein the activity level correlates to inhibition of PARG. In another aspect, the compound of the present invention is capable of conferring about 5% to about 80%, or about 20% to about 50%, protection in a cell survival assay wherein U937 cells are treated with hydrogen peroxide.

In an embodiment, the invention provides compounds and methods for inhibiting PARG activity in a target cell. The compound can be any of the structures disclosed herein, including structures FX1-FX4, or PIP-1. In an aspect, the compound to be administered for inhibiting PARG activity in a target cell is structure 11, 12, 13, 19-21, 23, or 37. The target cell can be a neurodefective cell. The cell can be within an organism (e.g. in vivo) or a whole cell isolated from the organism (e.g. in vitro). The target cell can be a neuronal cell or a neurodegenerative cell, including a cell from a human with Parkinson's disease, or from an animal model that mimics Parkinson's disease, or a cell suffering from an adverse biological event, including ischemia and stroke.

In an aspect, any of the compounds disclosed herein do not substantially inhibit a PARP molecule in a target cell. In an embodiment, any of the compounds disclosed herein do not inhibit a PARP-1 molecule in a target cell.

In another aspect, the invention is a method of treating a neurodegenerative condition in a patient comprising administering a compound that selectively inhibits PARG, including any of the compounds disclosed herein, or a compound having a chemical structure given by FX1, FX4 or PIP-1, or derivatives of PIP-1.

In an embodiment, the invention provides compounds and methods involving effective concentrations preferably from about 10 nM to about 100 μM of the disclosed structural formulas. In another preferred embodiment, the effective concentrations are from about 200 nM to about 5 μM. In an embodiment, the effective concentration is considered to be a value such as a 50% activity concentration in a direct PARG inhibition assay, in a cell survival assay, or in an animal clinical therapeutic assessment. In a preferred embodiment, such value is less than about 200 μM. In a preferred embodiment, the value is less than about 10 μM.

Compounds of the invention and compounds useful in the methods of this invention include those of the disclosed formulas and salts and esters of those compounds, including preferably pharmaceutically-acceptable salts and esters.

In an embodiment, the invention provides prodrug forms of compositions. Prodrugs of the compounds of the invention are useful in the methods of this invention. Any compound that will be converted in vivo to provide a biologically, pharmaceutically or therapeutically active form of a compound of the invention is a prodrug. Various examples and forms of prodrugs are well known in the art. A biomolecule such as a precursor protein or precursor nucleic acid can be a prodrug. Examples of prodrugs are found, inter alia, in Design of Prodrugs, edited by H. Bundgaard, (Elsevier, 1985), Methods in Enzymology, Vol. 42, at pp. 309-396, edited by K. Widder, et. al. (Academic Press, 1985); A Textbook of Drug Design and Development, edited by Krosgaard-Larsen and H. Bundgaard, Chapter 5, “Design and Application of Prodrugs,” by H. Bundgaard, at pp. 113-191, 1991); H. Bundgaard, Advanced Drug Delivery Reviews, Vol. 8, p. 1-38 (1992); H. Bundgaard, et al., Journal of Pharmaceutical Sciences, Vol. 77, p. 285 (1988); and Nogrady (1985) Medicinal Chemistry A Biochemical Approach, Oxford University Press, New York, pages 388-392).

In an embodiment, the invention provides a therapeutic composition comprising one or more compounds and for each compound a pharmaceutically acceptable salt or ester thereof; wherein the compounds are present in the composition in an amount or in a combined amount effective for obtaining the desired therapeutic benefit. The therapeutic compositions of this invention optionally further comprise one or more pharmaceutically acceptable components, for example carriers and excipients as known in the art.

In an aspect, the invention is a method for making any of compounds disclosed herein, comprising the schemes outlined in scheme 1, FIG. 12 or FIG. 23 and described herein.

Another embodiment of the invention is a screen for identifying compounds capable of inhibiting PARG and/or PARP. In an aspect, the screen involves contacting an isolated whole cell, or whole cell lysate, with an inducer of the PARG and/or PARP enzyme, including for example, an inducer of cell death. The cell or cell lysate is contacted with or without a test compound. The PARG and/or PARP enzyme is then activated and the activity of the enzyme measured. One embodiment of the screen is summarized in FIG. 14 and comprises lysing cells having PARG and/or PARP enzymes, adding NAD⁺ to the lysate along with an inducer. The inducer can be H₂O₂. The compound to be screened is added to the lysate, either before, after, or simultaneous to the inducer. The amount of NAD⁺ is then measured, for example by the fluorescent methodology disclosed herein or by other methods known in the art. A compound is identified as a PARG and/or PARP inhibitor if there is at least a measurable decrease in the amount of NAD⁺ depletion, compared to the level of depletion for lysate or cells not exposed to the compound.

### BRIEF DESCRIPTION OF THE DRAWINGS

FIG. 1: Schematic illustrating various pathways postulated to play an integral role in neuronal cell death in Parkinson's disease. The invention targets poly(ADP-ribose) activity to inhibit neuronal cell death.

FIG. 2A-C: Schematic illustration of poly(ADP-ribosyl)ation in response to DNA damage. A. In response to mild DNA damage, PARP-1 binds to DNA and catalyzes the formation of poly(ADP-ribose) polymers onto protein acceptors including itself. Due to electrostatic repulsion between the damaged DNA and the newly formed polymer, PARP-1 releases the DNA, which is then primed to recruit DNA repair enzymes such as αXRCC1 and DNA ligase. PARP-1 remains in an inactive state until the enzyme PARG degrades the poly(ADP-ribose) polymer, which allows it to once again bind damaged DNA. B. Upon more severe DNA damage, the cell initiates the apoptotic cascade. Release of cytochrome c ultimately leads to cleavage of PARP-1, thus preventing unnecessary cycles of DNA repair to save cellular energy for apoptosis. C. Extreme DNA damages causes PARP-1 overactivation, which results in a loss of cellular energy. With NAD⁺ severely depleted, the cell cannot make ATP, and this leads to inflammation and death through necrosis.

FIG. 3: Schematic diagram of proposed mechanism of the small-molecule PARG inhibitors of the present invention that inhibit PARG without significantly inhibiting PARP-1.

FIG. 4: Domain structure of PARG110, PARG103 and PARG99. Human PARG110 has a two-domain structure, with both a regulatory domain (1-417) and a catalytic domain (418-976). Along with a nuclear export signal (NES) from 126-134, a bipartite-type nuclear localization signal (NLS) is found from 421-426, along with putative classical-type NLS signals from 10-162 and 838-844.89 PARG 103 lacks exon 1, and PARG 99 lacks exons 1 and 2, which encode for the first NLS.

FIG. 5: Schematic illustrating the action of PARP and PARG. PARP catalyzes the formation of poly(ADP-ribose) polymers (from NAD⁺) on glutamic acid residues of protein substrates. This process can occur from either the 2′ position (elongation) or 2″ position (branching). PARG catabolizes the ADP-ribose polymers in both an exo- and endo-glycosidic fashion. ADP-ribose monomers are directly produced from exoglycosidic activity, whereas the endoglycosidic mode of action gives smaller ADP-ribose polymers that can be converted into ADP-ribose through further processing.

FIG. 6: Conversion of ADP-ribose to a fluorescent dye with benzamidine.

FIG. 7: Intercalator and intercalator-like inhibitors of PARG and their IC₅₀ values.

FIG. 8: Tannins and their representative IC₅₀ values.

FIG. 9: Substrate analogue PARG inhibitors and their comparative IC₅₀ values.

FIG. 10A-B: (A) PIP-1 inhibits PARG in vitro with an IC₅₀=0.49 uM. (B) PIP-1 protects differentiated PC-12 cells (and SK-N-SH cells, see FIG. 20) from MPP⁺ toxicity.

FIG. 11: PIP-1 derivative structures.

FIG. 12: Scheme for rapid synthesis of derivatives of PIP-1.

FIG. 13: Effects of each of PIP-1 and structures 1-26 on survival of U-937 cells treated with H₂O₂.

FIG. 14: A. Chemical reaction summary for quantifying NAD⁺ levels in vitro and in cell-based assays. The amount of product is measured fluorometically with excitation light at about 372 nm and emission measured at about 444 nm. B. The technique outlined in A can be incorporated into a whole cell system to obtain a screen for PARG and/or PARP inhibitors.

FIG. 15: A. About 22,000 small molecules are screened in a two-tiered system to identify inhibitors of PARP and/or PARG. The initial high-throughput screen identified 38 compounds. These compounds are then screened using standard PARP and standard PARG assays to identify PARG-specific inhibitors. B. The structures of three PARG inhibitors identified from the screen. C. The percent inhibition of NAD⁺ depletion in the whole cell assay screen at 50 μM by the identified PARG inhibitors. The identified inhibitors did not inhibit PARP-1 in vitro at concentrations up to 100 μM and the IC₅₀ values for PARG activity in vitro are determined by quantitation of free ADP-ribose monomers as described herein. The “Intercalator” column refers to whether the compound intercalates with DNA.

FIG. 16: Whether a compound intercalates with DNA is determined by analyzing the absorbance spectra. A shows the absorbance spectra for compound 2 and B shows the spectra for PIP-1. The ability to intercalate into DNA is determined by monitoring the change in absorbance spectra upon DNA addition to compound. The shift in spectra in A with increasing DNA concentration indicates compound-DNA intercalation. No such shift is observed for PIP-1.

FIG. 17: Toxicity screen of the compound 2 identified as a DNA intercalator in FIG. 16. The data shows cell death increases with compound concentration, suggesting the compound is toxic to cells.

FIG. 18: A. PIP-1 inhibits PARG in vitro in a dose dependent manner. B. PIP-1 is a cell permeable PARG inhibitor. PC-12 cells are pre-treated with PIP-1 then exposed to H₂O₂ to induce PARP activity. Cells are then fixed in ethanol, stained with an anti-poly(ADP-ribose) antibody and the amount of poly(ADP-ribose) present in each cell is determined using a fluorescently labeled secondary antibody and flow cytometry.

FIG. 19: PIP-1 and lactam 4 both inhibit NAD⁺ depletion in the whole cell assay, inhibit PARG activity in vitro, and protect neuronally-differentiated PC-12 cells from rotenone treatment. However, other structurally similar analogues 5 and 6 that do not inhibit NAD⁺ depletion or PARG activity, do not protect rotenone-treated, neuronally-differentiated PC-12 cells.

FIG. 20: A PIP-1 protects neuronally differentiated PC-12 cells from MPP⁺ (EC₅₀=1.81 μM) and rotenone (EC50=5.36 μM) induced toxicity. B. PIP-1 protects neuronally differentiated SK-N-SH cells from MPP⁺ (EC50=1.64 μM) and rotenone (EC₅₀=2.18 μM) induced toxicity. C. PIP-1 protects dopaminergic neurons from rotenone induced cell death. Neuronal cells from the midbrain of embryonic rats are isolated and treated with rotenone and a range of concentrations of PIP-1. Cells are stained with antibodies to MAP2 and TH to determine the proportion of dopaminergic neurons remaining. PIP-1 treated cells that are significantly different than the rotenone treated cells are denoted by asterisks (p-value<0.05). D. Compound 5 (see FIG. 19 for structure), a compound that is structurally similar to PIP-1 but does not inhibit PARG, does not significantly protect isolated dopaminergic neurons from rotenone induced cell death. The data in panels C and D are presented as ‘% TH⁺ and MAP2+ cells’ to normalize for differences in cell plating density from one slide to another. The approximately 3-fold decrease in the relative number of TH-positive neurons upon treatment with 100 nM rotenone resulted from a 6-fold decrease in the absolute number of TH-positive neurons, compared to only a 2-fold decrease in the absolute number of MAP2-positive neurons. Strikingly, PIP-1 has a more pronounced protective effect on the dopaminergic neurons compared to the total (MAP2 positive) neurons in rotenone-treated primary cultures. The negative-control compound 5 has no protective effect on either the dopaminergic or MAP2 positive neurons.

FIG. 21: A Blood-brain-barrier penetrance of PIP-1. B-D PIP-1 protects dopaminergic neurons in a mouse model of Parkinson's disease as assessed by the presence of dopamine and dopamine metabolites. C57 Black mice are given a single i.p. injection of varying amounts of PIP-1 and a single i.p. injection of MPTP (30 mg/kg). After 30 days, the mice are sacrificed, the striata are dissected, and HPLC used to determine the amount of dopamine and the dopamine metabolic products DOPAC and HVA. PIP-1/MPTP treatments that are significantly different than the MPTP treated mice are denoted by asterisks (p-value<0.05). E. PIP-1 has no effect on serotonin levels. The differences between the controls, PIP-1 treated mice, and PIP-1/MPTP treated mice are not statistically significant in this experiment (p-value<0.05).

FIG. 22: A. Scheme for synthesis of PIP-1 and compounds 5 and 6 (see FIG. 19 for structures). (i) Pd(OAc)₂, BINAP, t-BuOK, Toluene; (ii) 10% HCl, 100° C., 6 h; (iii) acetyl chloride, 40° C., 3 h; (iv) dimethylamine, THF, 140° C., Microwave; (v) NaOEt, EtOH, reflux, 6 h. B. Synthesis of 4-(dimethylamino)-3-amino-2-phenylpyridinone, compound 4. (a) HNO₃, Δ; (b) cycloheptyl amine, MeOH, POCl₃, 25° C.; (c) dimethylamine, THF, A; (d) phenylboronic acid, Cu(OAc)₂, molecule sieve, 25° C.; (e) Na₂S₂O₄, dioxane/water/NH₄OH.

### DETAILED DESCRIPTION OF THE INVENTION

All references cited herein are hereby incorporated by reference to the extent not inconsistent with the disclosure herewith. The following definitions are provided to clarify their specific use in the context of the invention.

When used herein, the term “inhibit PARG” or “inhibiting PARG” refers to a compound that measurably inhibits the activity of PARG as measured by an experimental assay known in the art, including the assay of Putt and Hergenrother (2004) and may inhibit PARG activity by at least 10%, at least 25%, at least 50%, at least 75%, or at least 90%, and all ranges between 10% and 100%. In an embodiment the IC₅₀ (e.g. concentration required for 50% PARG inhibition) value of the PARG inhibitor may be less than about 100 μM, less than about 10 μM, less than about 3 μM, or less than about 1 μM. The PARG activity can also be assessed using whole cell assays wherein the EC₅₀ (e.g. concentration required for 50% inhibition of cell death) for a PARP inhibitor may be less than about 100 μM, less than 50 μM, less than 25 μM, less than 10 μM, less than 5 μM, or less than about 2 μM. PARG activity can also be assessed using whole animal models.

When used herein, the term “PARG inhibitor” refers to any substance capable of inhibiting PARG, and includes substances that can reduce or prevent the death of target cells as compared to the death rate of target cells not exposed to the PARG inhibitor. In an embodiment the PARG inhibitor refers to any substance capable of reducing or preventing the death of neurological cells compared to neurological cells not exposed to the PARG inhibitor. In an embodiment the neurological cells are contained in a patient suffering from a neurodegenerative disease, including Parkinson's disease, as well as from a patient suffering from an ischemic event, including stroke. “Target cell” is used broadly herein, and refers to cells whose survival improves when PARG is inhibited. In the case of cancer cells as target cells, however, PARG inhibitors can function so as to decrease the survival rate of these cancerous target cells. A compound is said to “not substantially inhibit a PARP molecule” when the IC₅₀ value for PARP is at least ten times greater than the IC₅₀ value for PARG (e.g., greater than 100 μM for PARP inhibition and less than 10 μM for PARG).

When used herein, the term “chemotherapeutic agent” refers to any substance capable of reducing or preventing the growth, proliferation, or spread of a cancer cell, a population of cancer cells, tumor, or other malignant tissue. The term is intended also to encompass any antitumor or anticancer agent. In an embodiment the PARG inhibitor is a chemotherapeutic agent.

When used herein, the term “neurodegenerative defective cell” refers to a neurological cell suffering from a biological challenge, wherein the challenge results in a decrease in survivability and/or function of the neurological cell. Accordingly, the term encompasses cells suffering from a neurodegenerative disease such that the patient is, or is predisposed to, neurological impairment. The term also encompasses challenges arising externally from the cell, including ischemic (and reperfusion) events wherein decrease in oxygen and/or sudden reperfusion results in an adverse challenge to the neurological cells, thereby resulting in increased cell death.

When used herein, the term “effective amount” is intended to encompass contexts such as a pharmaceutically effective amount or therapeutically effective amount. For example, in embodiments the amount is capable of achieving a beneficial state, beneficial outcome, functional activity in a screening assay, or improvement of a clinical condition.

When used herein, the term “cancer cell” is intended to encompass definitions as broadly understood in the art. In an embodiment, the term refers to an abnormally regulated cell that can contribute to a clinical condition of cancer in a human or animal. In an embodiment, the term can refer to a cultured cell line or a cell within or derived from a human or animal body. A cancer cell can be of a wide variety of differentiated cell, tissue, or organ types as is understood in the art.

The term “alkyl” refers to a monoradical branched or unbranched saturated hydrocarbon chain preferably having from 1 to 22 carbon atoms and to cycloalkyl groups having one or more rings having 3 to 22 carbon atoms. Small alkyl groups are those having 1 to 6 carbon atoms including methyl, ethyl, propyl, butyl, pentyl and hexyl groups, including all isomers thereof. Long alkyl groups are those having 8-22 carbon atoms and preferably those having 12-22 carbon atoms as well as those having 12-20 and those having 16-18 carbon atoms. The term “cycloalkyl” refers to cyclic alkyl groups of from 3 to 22 carbon atoms having a single cyclic ring or multiple condensed rings. Cycloalkyl groups include, by way of example, single ring structures such as cyclopropyl, cyclobutyl, cyclopentyl, cyclohexyl, cyclooctyl, and the like, or multiple ring structures such as adamantanyl, and the like.

The term “alkenyl” refers to a monoradical of a branched or unbranched unsaturated hydrocarbon group preferably having from 2 to 22 carbon atoms and to cycloalkenyl groups having one or more rings having 3 to 22 carbon atoms wherein at least one ring contains a double bond. Alkenyl groups may contain one or more double bonds (C═C) which may be conjugated. Preferred alkenyl groups are those having 1 or 2 double bonds. Short alkenyl groups are those having 2 to 6 carbon atoms including ethylene (vinyl) propylene, butylene, pentylene and hexylene groups, including all isomers thereof. Long alkenyl groups are those having 8-22 carbon atoms and preferably those having 12-22 carbon atoms as well as those having 12-20 carbon atoms and those having 16-18 carbon atoms. The term “cycloalkenyl” refers to cyclic alkenyl groups of from 3 to 22 carbon atoms having a single cyclic ring or multiple condensed rings in which at least one ring contains a double bond (C═C). Cycloalkenyl groups include, by way of example, single ring structures such as cyclopropenyl, cyclobutenyl, cyclopentenyl, cyclooctenyl, cyclooctadienyl and cyclooctatrienyl.

The term “alkynyl” refers to a monoradical of an unsaturated hydrocarbon preferably having from 2 to 22 carbon atoms and having one or more triple bonds (C≡C). Alkynyl groups include ethynyl, propargyl, and the like. Short alkynyl groups are those having 2 to 6 carbon atoms, including all isomers thereof. Long alkynyl groups are those having 8-22 carbon atoms and preferably those having 12-22 carbon atoms as well as those having 12-20 carbon atoms and those having 16-18 carbon atoms.

The term “aryl” refers to a group containing an unsaturated aromatic carbocyclic group of from 6 to 22 carbon atoms having a single ring (e.g., phenyl), one or more rings (e.g., biphenyl) or multiple condensed (fused) rings, wherein at least one ring is aromatic (e.g., naphthyl, dihydrophenanthrenyl, fluorenyl, or anthryl). Aryls include phenyl, naphthyl and the like. Aryl groups may contain portions that are alkyl, alkenyl or akynyl in addition to the unsaturated aromatic ring(s). The term “alkaryl” refers to the aryl groups containing alkyl portions, i.e., -alkylene-aryl and -substituted alkylene-aryl). Such alkaryl groups are exemplified by benzyl, phenethyl and the like.

Alkyl, alkenyl, alkynyl and aryl groups are optionally substituted as described herein and may contain 1-8 non-hydrogen substituents dependent upon the number of carbon atoms in the group and the degree of unsaturation of the group.

The term “alkylene” refers to a diradical of a branched or unbranched saturated hydrocarbon chain, preferably having from 1 to 10 carbon atoms, more preferably having 1-6 carbon atoms, and more preferably having 2-4 carbon atoms. This term is exemplified by groups such as methylene (—CH₂—), ethylene (—CH₂CH₂—), more generally —(CH₂)ₙ—, where n is 1-10 or more preferably 1-6 or n is 2, 3 or 4. Alkylene groups may be branched. Alkylene groups may be optionally substituted. Alkylene groups may have up to two non-hydrogen substituents per carbon atoms. Preferred substituted alkylene groups have 1, 2, 3 or 4 non-hydrogen substituents.

The term “arylene” refers to the diradical derived from aryl (including substituted aryl) as defined above and is exemplified by 1,2-phenylene, 1,3-phenylene, 1,4-phenylene, 1,2-naphthylene and the like.

The term “amino” refers to the group —NH₂ or to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl and heterocyclic provided that both R's are not hydrogen.

Alkyl groups are optionally substituted as discussed herein and may, dependent upon the size of the alkyl group, have preferably from 1-10 substituent groups. Substituted alkyl groups include those that carry 1 to 8 substituents, 1 to 5 substituents, 1 to 3 substituents, and 1 or 2 substituents.

“Haloalkyl” refers to alkyl as defined herein substituted by one or more halo groups as defined herein, which may be the same or different. Representative haloalkyl groups include, by way of example, trifluoromethyl, 3-fluorododecyl, 12,12,12-trifluorododecyl, 2-bromooctyl, 3-bromo-6-chloroheptyl, and the like.

The term “heteroaryl” refers to an aromatic group of from 2 to 22 carbon atoms having 1 to 4 heteroatoms selected from oxygen, nitrogen and sulfur within at least one ring (if there is more than one ring). Heteroaryl groups may be optionally substituted

The term “heterocycle” or “heterocyclic” refers to a monoradical saturated or unsaturated group having a single ring or multiple condensed rings, from 2-22 carbon atoms and from 1 to 6 hetero atoms, preferably 1 to 4 heteroatoms, selected from nitrogen, sulfur, phosphorus, and/or oxygen within at least one ring. Heterocyclic groups may be substituted.

The term “ester” refers to chemical entities as understood in the art and in particular can include groups of the form (RCO—).

As to any of the above groups which contain one or more substituents, it is understood, that such groups do not contain any substitution or substitution patterns which are sterically impractical and/or synthetically non-feasible. The compounds of this invention include all novel stereochemical isomers arising from the substitution of disclosed compounds.

The invention may be further understood by the following non-limiting examples.

### EXAMPLE 1

### Small Molecule Inhibition of PARG

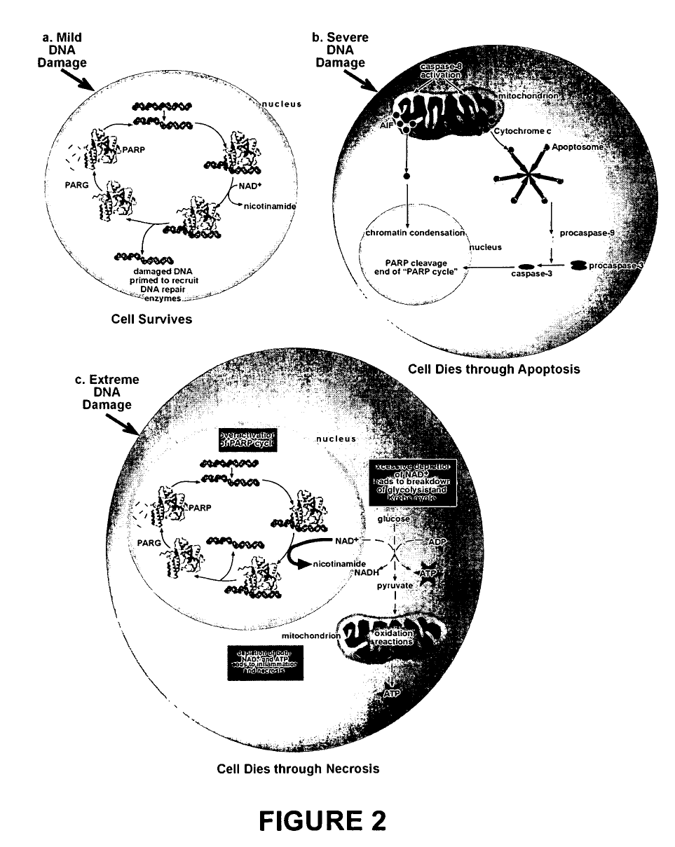

Driven by DNA damage, the poly(ADP-ribose) polymerases (PARPS) utilize NAD⁺ to create poly(ADP-ribose) (PAR) on the glutamic acid residues of proteins, drastically altering the overall charge and size of the modified protein and ultimately initiating DNA repair.¹ PAR biopolymers consist of up to 200 ADP-ribose units, and polymer levels may increase more than 100-fold in minutes.² This poly(ADP-ribosyl)ation of proteins is transient, as the enzyme poly(ADP-ribose)glycohydrolase (PARG) operates in both an endo- and exoglycosidic fashion to break down PAR into ADP-ribose monomers.³ If more severe DNA damage occurs, PARP-1 becomes cleaved by caspases into 89- and 24-kDa subunits,^{4,5} which separates the DNA binding domain from the automodification and catalytic domains of PARP, thus inactivating the enzyme. Most likely this PARP-1 cleavage plays a role in preserving cellular energy for apoptosis by preventing futile cycles of DNA repair.⁶ Finally, in cases of extreme DNA damage PARP-1 is overactivated, which depletes the cell's valuable energy resources, thus leading to death by necrosis.⁷ The cellular activity and processing of PARP-1 in response to these three levels of DNA damage is depicted in FIG. 2. While PAR has been shown to play a role in DNA repair and replication,⁸ it has also been implicated in control of chromosome migration during cell division,⁹ activation of transcription after DNA damage,¹⁰ activation of the proteasome,¹¹ and regulation of telomere maintenance¹² and is required for spindle assembly and function.¹³ Failure to dynamically regulate PAR metabolism causes increased cytotoxic sensitivity and leads to early embryonic lethality.¹⁴

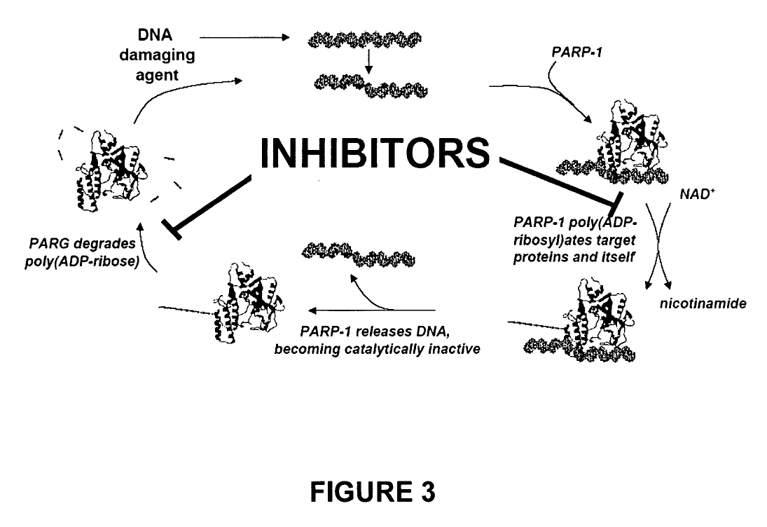

Due to these key roles of PAR in the cell, the PARP isozymes and PARG are potential drug targets (FIG. 3). Overactivation of PARP caused by severe DNA damage quickly leads to the depletion of NAD⁺, and this loss of energy inside the cell usually culminates in cell death through necrosis.⁷ In cases of ischemic injury, when oxygen deprivation alone drastically decreases cellular energy output, overstimulation of PARP can also cause a drop of NAD⁺ and ATP to less than 20% of the normal level.¹⁵ Inhibition of either PARP or PARG in such cases could maintain cellular energy at acceptable levels, thus decreasing necrotic cell death. Conversely, when utilized in the context of tumor cells, PARP or PARG inhibitors may enhance the cytotoxicity of cancer therapies already in use.¹⁶ The addition of such an inhibitor prevents the recovery of tumor cells from lethal DNA damage after radiation or other cancer therapies. The requirement for PAR during spindle assembly¹³ suggests utility of spindle-associated PARP and PARG as cancer drug targets. Finally, both PARP and PARG are potential targets for neuroprotection. Just as inhibition of these enzymes can alleviate damage in cases of ischemic injury, inhibition of PARP and PARG can reduce neuronal death.¹⁷ Additionally, although it is unknown if abnormal accumulation of PAR is found in humans, a number of neurodegenerative disorders such as Alzheimer's and Parkinson's diseases are associated with the accrual of protein aggregates, and study of PAR accumulation in cases of PARG inhibition can be useful in further understanding these conditions.¹⁸ Clearly, PAR's pivotal role in the cell indicates that both PARP and PARG inhibitors are promising drug targets of medicaments (FIG. 2).

Disadvantages of PARP inhibitors. Although they have limited cellular uptake and low potency and specificity, the most commonly used PARP inhibitors are nicotinamide, the endogenous inhibitor of PARP, and 3-aminobenzamide.¹⁹⁻²² Many other PARP inhibitory compounds fall into two classes: monoaryl amides and bi-, tri-, or tetracyclic lactams, which often contain either an aromatic carboxamide or a carbamoyl group attached to a polyaromatic heterocyclic skeleton.¹⁹ To date, the PARP family comprises at least 18 isozymes,²³ and due to the highly conserved NAD⁺ binding site of these PARPs, isozyme-specific inhibitors will be difficult to develop.²⁴ Although some preliminary studies have been completed that compare the inhibitory effects of phenanthridinones on PARP-1 versus PARP-2,²⁵ the issue of isozyme specificity has not been fully examined. While it cannot be denied that PARP inhibitors have been shown to have a beneficial effect in many in vivo studies,^{19,26,27} before they can be clinically utilized some important safety aspects must first be considered. Although PARP-deficient mice do not show an increased level of spontaneous tumors, they do display an increased number of chemically induced tumors in comparison to wild-type ones,²⁸ and even the most well-documented PARP inhibitors display undesired metabolic side effects by knocking out the normal physiologic functions of PAR.^{29,30} Long-term studies have not yet been completed, but due to PARP's active roles in DNA repair and preservation of genomic integrity,^{8-10,12,13} extended PARP inhibition could increase mutation rates and ultimately lead to cancer.¹⁹

PARG inhibition. Obstruction of DNA repair mechanisms by PARP inhibition can be avoided by inhibiting PARG instead of PARP. Although cellular PARG is 13-50 times less than that of PARP (it has been reported that the number of PARG molecules present per cell may be as low as 2000), inhibitors of PARG can be just as useful as any previously demonstrated PARP inhibitors; in fact, the specific catalytic activity of PARG is 50- to 70-fold higher than that of PARP.^{31,32} While PARP inhibition prevents the formation of PAR and consequently disrupts its normal metabolic functions, PARG inhibition indirectly slows PARP activity by locking PARP in the fully automodified (with PAR) and inactive state. PARG inhibition can allow PAR to maintain its normal function within the cell.^{29,30} Finally, in stark contrast to the burgeoning PARP family of enzymes there has only been one PARG identified to date,²³ and thus its inhibition would not be complicated by the need for isozyme specificity. By searching for direct inhibitors of PARG enzymatic activity, potent, specific, and pharmacologically acceptable drugs can be discovered, including small molecules described herein.

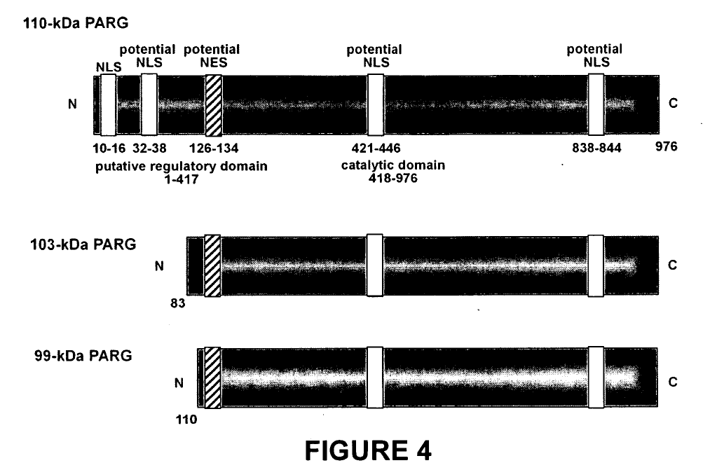

There are at least 18 proposed members of the PARP family,²³ but only one PARG has been identified to date, and its sequence is highly conserved among mammals.³ Three PARG isoforms are known: the 110-kDa nuclear form, and the more abundant 103- and 99-kDa cytoplasmic forms^{2,33} (FIG. 4). Homology searches indicate that the PARG sequence contains both a nuclear localization signal (NLS)³⁴ and a leucine-rich nuclear export signal (NES),³⁵ suggesting that PARG shuttles between the cytoplasm and nucleus to maintain tight control of PAR catabolism (FIG. 4).³³ Interestingly, PARG is a substrate for caspase-3, but the functional implications of this cleavage are unclear to date.³⁶ Although it is stable between pH 5 and 9, PARG's optimum pH for enzymatic activity has been found to be 7.0 to 7.5. The enzyme exhibits substantial thermostability, and its activity increases up to temperatures of 50° C.; thereafter, PARG activity decreases sharply.³¹

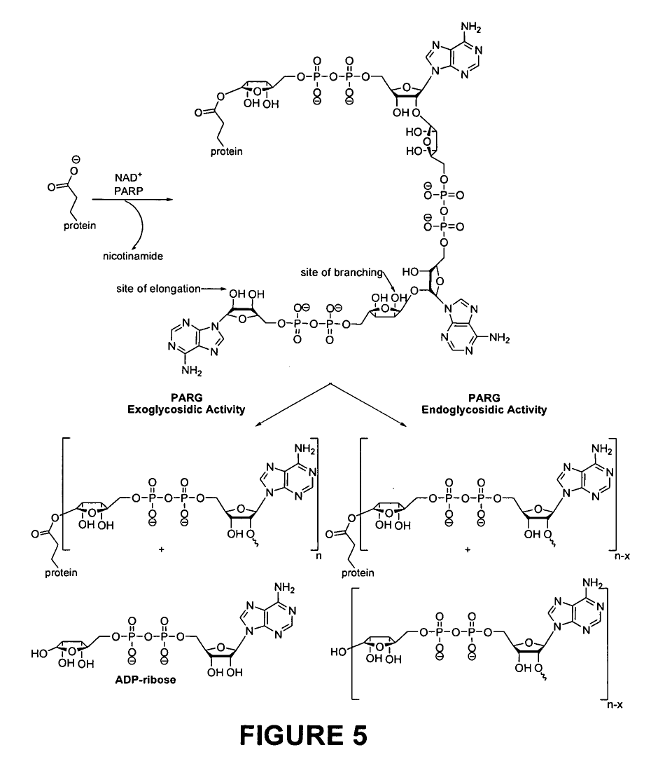

As highlighted earlier, although PARG has a low cellular concentration, it is constitutively active and has a high specific activity (5400 turnovers per minute);³¹ as such, the in vivo half-life of PAR in DNA-damaged cells is only ˜1 minute.³⁷ The nuclear PAR concentration is approximately one to two times the K_{M} of PARG (0.3 μM),^{3,31} and it has been found that the rates of PAR hydrolysis are a function of both concentration and structure.³⁸ Long chain polymers are catabolized more quickly, leaving behind 20- to 35mers, which are poorer substrates for PARG.^{34,39} In early stages of PAR catabolism, the enzyme degrades larger polymers in both an endo- and exoglycosidic fashion (FIG. 5), leaving behind both ADP-ribose monomers and smaller polymers, usually less than half the size of the original polymer. Conversely, smaller PAR are degraded at a rate that is 20-fold slower (for larger PAR the K_{M} is approximately 100-fold lower than that of small polymers), suggesting that PARG differentially controls the levels of large and small PAR.³¹ Interestingly, it is known that binding of photolabeled adenosine diphosphate (hydroxymethyl)pyrrolidinediol (APD-HPD), an ADP-ribose analogue inhibitor of PARG, competes with the binding of short PAR (5- to 15mer) but not with the longer, highly branched polymer.⁴⁰ For PARG, substrate identification and binding is a complex event, with possible multiple attachment points or subsites for polymer recognition. It also must be noted that these striking differences in substrate processing may be attributed to higher-order structures of large PAR; those polymers with a chain length of more than nine ADP-ribose units exhibit a secondary structure, while shorter chain length polymers do not.⁴¹

No crystal structure for PARG has yet been reported. However, from studies of the catalytic fragment of recombinant bovine PARG, it is believed that the active site consists of at least some residues in the Leu-771 to Arg-801 region. Photoderivatization studies with a known PARG inhibitor indicate Tyr-796 (Y796), which is a highly conserved residue in PARGs across a wide range of organisms, as a possible amino acid important for initial binding of PAR. Site-directed mutagenesis to replace Y796 with an alanine residue (Y796A) leads to an 8-fold decrease in catalytic efficiency (K_{cat}/K_{M}), a difference attributed to the ring-stacking ability of the Y796 residue with the adenine of the PAR substrate.^{42,43} From homology studies it is known that Gly-262 and Gly-263 in Arabidopsis PARG are found in all mammalian PARGs, and a third Gly-264 is highly conserved.⁴⁴ When Gly-262 is replaced with a glutamic acid residue to create the G262E mutant PARG enzyme, Arabidopsis seedlings accumulate much higher levels of PAR as compared to wild-type. These data, together with the high degree of amino acid conservation in this region of the PARG sequence, suggest that this GGG motif plays an important role in the regulation of PARG activity.⁴⁴

Mutation in the PARG gene, tej, in plants has been demonstrated to influence the transcription of genes involved in circadian rhythm regulation. Using a cycling bioluminescence reporter phenotype, tej mutants with abnormal circadian oscillations were discovered in Arabidopsis thaliana. While the effects of this recessive tej mutation are independent of light input, tej plants exhibit a period length 2 hours longer than the wild-type and always flower earlier under both long- and short-day conditions. Interestingly, PARP inhibitors rescue the mutant tej phenotype. By slowing or stopping the rate of PAR synthesis, the known PARP inhibitor 3-aminobenzamide causes tej mutants to exhibit a wild-type circadian rhythm, thus indicating that poly(ADP-ribosyl)ation establishes a period length of the Arabidopsis circadian oscillator.⁴⁴

To date few studies have been completed with PARG knockout organisms. Loss of the catalytic domain of PARG causes lethality under normal conditions for development (25° C.) in the larval stages of Drosophila melanogaster, but if the incubation temperature is increased to 29° C., approximately 25% of the PARG knockouts survive to the adult stage¹⁸. Organisms lacking PARG have excessive accumulation of PAR in the central nervous system (CNS), progressive neurodegeneration, reduced locomotor activity, and shortened lifespan. Accumulation of PAR in the CNS can be explained by any of the following: PAR cannot be diluted in the CNS because neuronal cells in the CNS do not divide; PARP is possibly more active in the brain, therefore polymer builds up more quickly there;⁴⁵ or the other enzymes responsible for degrading PAR are less active in the brain than in other organs of D. melanogaster. Whatever the reason for the excessive amounts of PAR found in the CNS of these PARG knockouts, these studies indicate that PAR metabolism is a necessary part of normal neuronal function.

Through gene targeting in embryonic stem cells and in mice, deletion of the 110-kDa PARG protein, which is normally found in the nucleus,² has been thoroughly studied. While no full-length PARG mRNA is detected in the PARG knockout embryonic fibroblasts, a 60-kDa truncated version of PARG remains present.⁴⁶ Mice deficient in the full-length PARG do not show any obvious phenotype, are fertile, and do not exhibit excessive buildup of PAR in tissue as might first be expected. This is attributed to the fact that the 60-kDa PARG remains unaffected in such cases and can still be observed by Western blot analysis. On the other hand, specific PARG activity is significantly lower in cells obtained from 110-kDa PARG-deficient animals, and they are hypersensitive to DNA alkylating agents, radiation, lipopolysaccharide-induced septic shock, and streptozotocin-induced diabetes.⁴⁶ Accordingly, PARG inhibition can be used in concert with other cancer treatment regimens (e.g. radiation, chemotherapy) to improve treatment outcome.

In vivo studies indicate that catabolism of PAR by PARG is necessary for genomic stability in higher order eukaryotes.¹⁴ In contrast to the above study in which PARG activity remained in the 110-kDa PARG knockout mice due to the presence of alternatively spliced isoforms, complete PARG knockouts are lethal. While PARG^{+/-} mice are viable and fertile, homozygous null mice (PARG^{-/-}) arrest before gastrulation at embryonic day 3.⁵ Under conditions of PARP inhibition (treatment with the PARP inhibitor benzamide), PARG^{-/-} embryonic trophoblast stem (TS) cells can be cultured, although removal of the PARP inhibitor causes abnormal morphology, and after 3 days approximately 60% of the PARG^{-/-} TS cells undergo apoptosis.¹⁴ Cells remaining after removal of the benzamide PARP inhibitor also exhibit slow growth rates, PAR accumulation, and increased sensitivity to the cytotoxic agent MNNG. Due to the lethality of the knockouts, it is difficult to study PARG via traditional genetic approaches.

PARG purification and assays. Although provocative data indicate PARG inhibitors can be useful for a range of maladies, the lack of detailed structural knowledge has hampered PARG inhibitor development. As a result, substrate analogues and compounds identified through random screening are the only known PARG inhibitors. However, this invention utilizes advances in both PARG production and high-throughput assays to discover PARG inhibitors. Described in this example are methods for obtaining the PARG enzyme, assay protocols, and a comprehensive examination of known PARG inhibitors.

Due to the very low content of PARG in various tissues and its instability during purification, it is difficult to obtain pure quantities of this enzyme. Since its initial discovery in 1971,⁴⁷ PARG has been purified and characterized from a wide range of cell types, which has allowed for full enzymatic characterization. Initial attempts at obtaining PARG focused on its extensive purification from calf thymus and other tissues, a process which can involve up to six purification steps and produces low yields of purified enzyme.^{31,41,48-50} Additionally, although PARG is known to have a molecular weight of about 110 kDa,^{34,51} during purification it is easily proteolyzed to 59- and 74-kDa fragments,⁵² which originally caused uncertainty as to whether different types of PARG protein exist in the cell.⁵³ However, full-length PARG was purified in 1997³⁴ utilizing recombinant expression from the 4.1-kb PARG cDNA in Escherichia coli to produce the 110-kDa protein, thus eliminating the controversy surrounding PARG's size.

PARG enzymatic activity assays. Many assays for measuring PARG enzymatic activity are limited by the fact that they are neither convenient nor high-throughput. The most common technique used to evaluate PARG activity first involves the preparation of ³²P-labeled PAR using PARP and ³²P-labeled NAD⁺. Then, following purification and reaction of the radiolabeled PAR with PARG, either a thin-layer chromatography (TLC) technique or high-resolution polyacrylamide gel electrophoresis is utilized to separate the catabolized ADP-ribose from PAR, and radioactivity is measured.^{54,55} Although both these radiometric assays have been helpful in determination of IC₅₀ values for PARG inhibitors^{42,56,57} and in studies of PARG kinetics,^{42,58} neither is particularly useful in screening large libraries of compounds in a high-throughput manner. Additionally, PARG works in an endo- and exoglycosidic fashion, and these TLC-based methods are only able to quantitate monomeric ADP-ribose; the direct products of endoglycosidic cleavage cannot be detected.⁵⁹

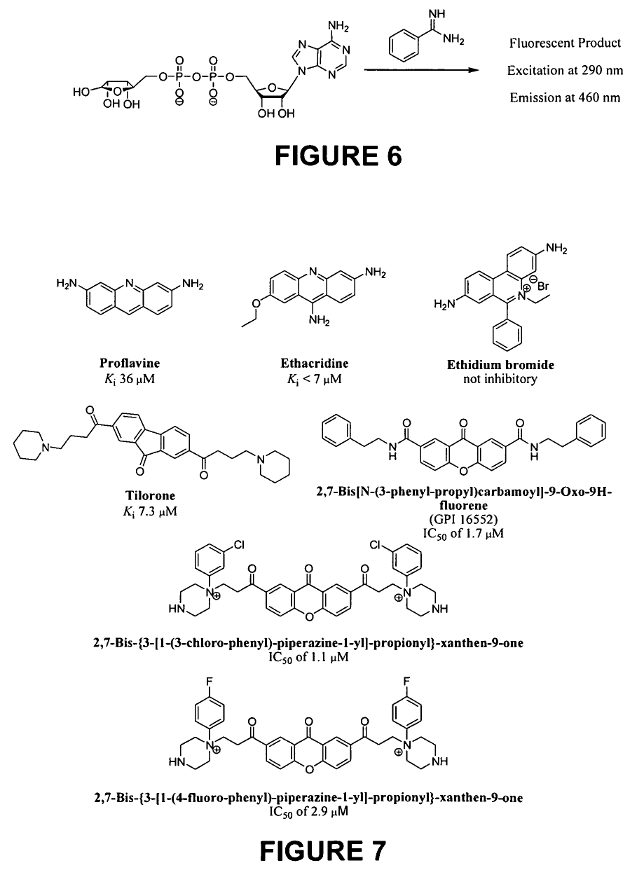

More recently, a nonradiometric assay involving the conversion of the ADP-ribose product of PARG activity into a fluorescent molecule has been developed. Taking advantage of the fact that reducing sugars are known to react with aromatic amidines to give fluorescent products,⁶⁰⁻⁶² this assay involves the reaction of benzamidine with ADP-ribose (a reducing sugar) to create a fluorophore (FIG. 6).⁵⁹ A simple fluorescence reading can be used to measure the amount of free ADP-ribose hemiacetal, thus quantifying PARG activity. One limitation of this assay is that it cannot be used in cases in which potential PARG inhibitors contain aldehydes or reducing sugars. Unlike the aforementioned PARG assays, this fluorescence-based method is capable of detecting both exo- and endoglycosidic cleavage of PAR, and this method is highly sensitive. Additionally, development of an assay based on fluorescence rather than radioactivity greatly reduces both cost and time expenditures and allows compounds to be screened in a high-throughput manner.

PARG inhibitors. From a drug-development perspective, an ideal PARG inhibitor would be highly potent, specific, and of modest size (molecular weight<500). Although the current classes of inhibitors do not necessarily fit into all the categories of an ideal pharmaceutical, they have certainly offered insights into the structure and function of PARG. These compounds can be broadly grouped into three categories: (i) DNA intercalators; (ii) tannins; and (iii) substrate analogues.

DNA intercalators. Of the few classes of PARG inhibitors known, the ones that potentially offer the best cell permeability are polyaromatic compounds typically thought of as DNA intercalators, depicted in FIG. 7. These compounds were initially evaluated as a consequence of their known DNA binding properties and the known role of PARG in the DNA damage and repair process.

Using the ³²P-PAR assay, the K_{I} for ethacridine was estimated at <7 μM, the K_{I} for Tilorone was ˜7 μM, and the K_{I} for profavine was 36 μM, while another well-known DNA intercalator, ethidium bromide, is not inhibitory.⁶³ These were all relative to a K_{M} for the PAR substrate that was calculated as 1.7 μM in this particular assay system. In general, these inhibitors appear to give competitive inhibition, and there is some evidence that this inhibition may be due to the compounds forming a complex with the PAR polymer, thus blocking substrate binding to the enzyme; this seems to be particularly true for ethacridine.^{63,64} Further support of this hypothesis is found in the fact that ethacridine and other intercalators not only inhibit the degradation of PAR by PARG, but also prevent degradation of the polymer by snake venom phosphodiesterase and hinder ethanol/acetate precipitation of PAR.⁶³ Recent work has focused on the design and synthesis of symmetrically disubstituted aromatic compounds that exhibit PARG-inhibitory properties.¹⁶ Representative members of two of the most potent classes, 2,7-substituted-fluorenes and -xanthen-9-ones, which are structurally very similar to the DNA intercalator Tilorone, are also depicted in FIG. 7. In conclusion, while these compounds are modestly potent inhibitors of PARG and have been used in several in vivo studies (see below), questions about their selectivity for PARG inhibition versus DNA binding are likely to doom their use as therapeutic agents or chemical tools to extensively investigate PARG biology.

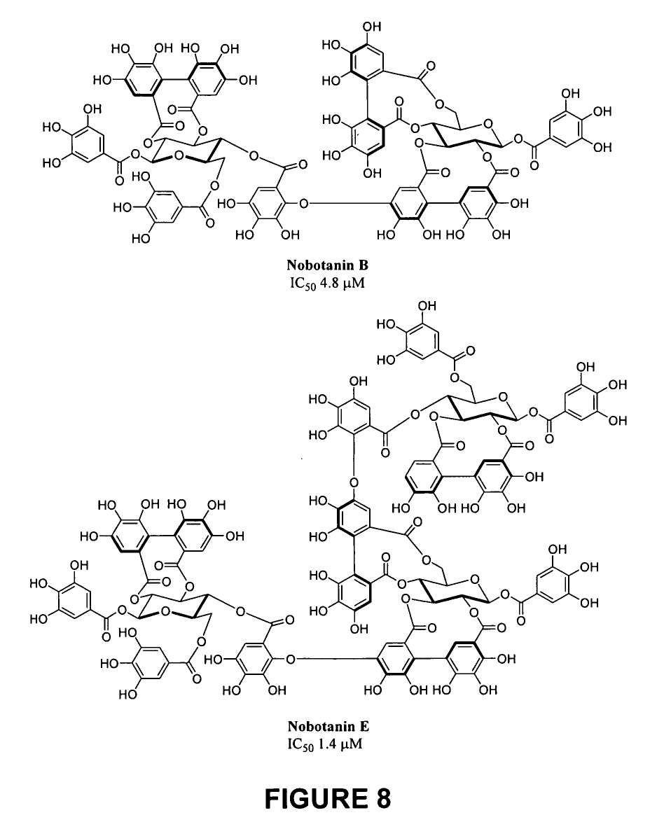

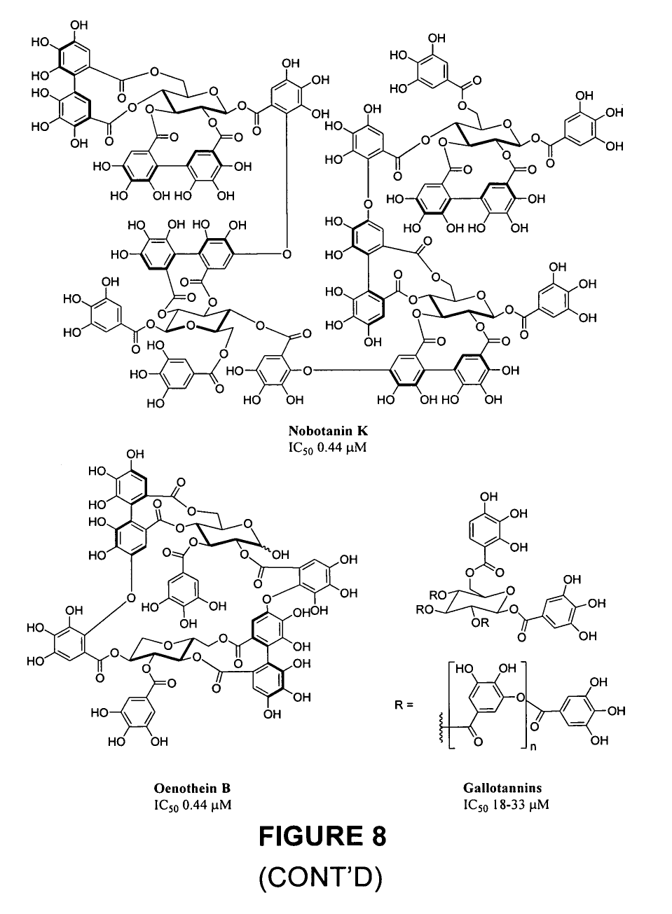

Tannins. The naturally occurring polymethoxy-phenolic compounds called tannins are by far the most well-studied PARG inhibitors (FIG. 8). Initially extracted from green tea leaves and pinecone fractions, it has been found that the oligomeric ellagitannins such as nobotanin B, E, and K are the most potent tannin inhibitors of PARG, with in vitro IC50 values ranging from 0.44 μM for nobotanin K to 4.8 μM for nobotanin B.⁶⁵ In comparison, the circular dimeric ellagitannin Oenothein B (Oen B) inhibits with an IC₅₀ of 3.8 μM.⁵⁸ Inhibitory activity of oligomeric ellagitannins increases with the number of monomeric residues (dimeric<trimeric<tetrameric), with condensed tannins having little effect on PARG activity even at very high concentrations, possibly indicating that differences in inhibitory effects are related to variations in secondary structures of the tannins.⁶⁶ Only hydrolyzable tannins (gallotannins and ellagitannins) were able to inhibit PARG. While tannins of the trimeric and tetrameric variety exhibit mixed-type inhibition, nobotanin B, (a dimer) has been found to competitively inhibit PARG with respect to PAR.⁶⁵

Several studies with cell extracts have confirmed the inhibitory properties of these phenolic phytochemicals against PARG. When 200 μM tannin was added to HeLa nuclear extracts, a significant 40-fold increase of the PAR level was observed, and it was confirmed that this increase was indeed due to inhibition of the catalytic activity of PARG.⁶⁷

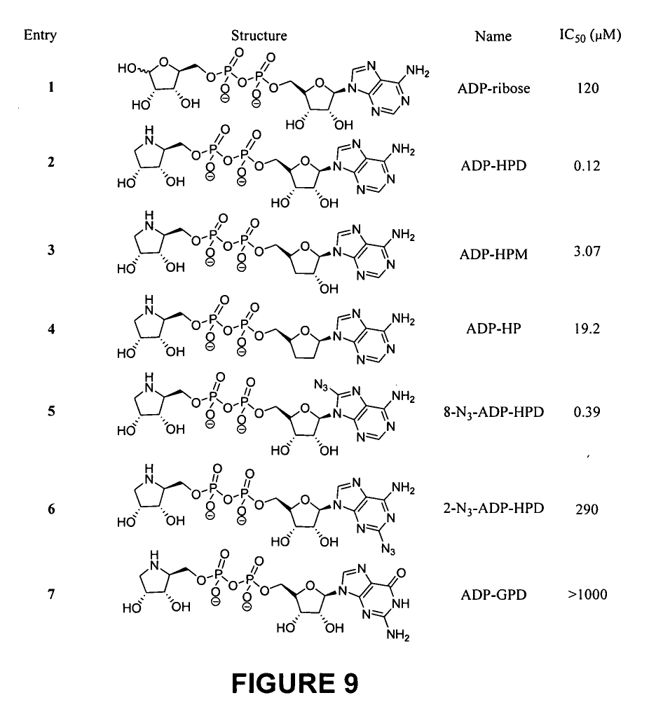

Substrate analogues. Substrate analogues of PAR have been synthesized and are shown to be both potent and specific inhibitors of PARG (FIG. 9). Though ADP-ribose monomer units inhibit PARG with an IC₅₀ of 120 μM, ADP-HPD, an NH-analogue of ADP-ribose, has been shown to inhibit the action of PARG with a 1000-fold lower IC₅₀ value (entry 2).⁵⁷ Structure-activity analysis utilizing ADP-HPD analogues containing various alterations to the purine base has revealed the importance of the adenine moiety for optimal inhibition. It was discovered that substitution at the 2-position of adenine negatively affects PARG inhibition (entry 7), while substitution at the 8-position has no adverse effects (entry 5). Both the pyrrolidine cis-hydroxyls of ADP-HPD are necessary for inhibitor binding; removal of one increases the IC₅₀ value to 3.07 μM (entry 3) and complete elimination of these hydroxyls results in a 160-fold increase in IC₅₀ value as compared to ADP-HPD (entry 4).⁴²

Other inhibitors. Additionally, through random screening it has been found that some cyclic peptides are capable of inhibiting PARG activity. One cyclic hexadepsipeptide (known as PD 124,966) isolated from the fermentation products of actinomycetes was found to have an IC₅₀ value of 40 μg/ml, while pargamicin, which is purified from the fermentation broth of Amicolatopsis exhibits an IC₅₀ value of 28 μg/ml.⁵⁶ Interestingly, linear peptides containing the same piperazic acid residues as PD 124,966 and pargamicin appear to have no effect on PARG activity.

In vivo studies. Studies with DNA intercalators. The second generation intercalator-like PARG inhibitors are ideal for in vivo studies due to their low molecular weights and good cell permeability properties. One member of the Tilorone family of PARG inhibitors, GPI 16552 or 2,7-Bis[N-(3-phenyl-propyl)carbamoyl]-9-Oxo-9H-fluorene, has exhibited neuroprotective properties in a rat model of focal cerebral ischemia. Both a 30-minute pre-ischemia treatment or a 1- or 2-hour post-ischemia treatment with GPI 16552 reduces the total infarct volume by 47-53%, with greater protection observed in the cortical areas (43-59%) than the subcortical areas (28-40%) of the brain.²⁹ Furthermore, application of the novel PARG inhibitor GPI 18214 substantially reduces the septic shock-like syndrome caused by zymosan in mice.⁶⁸ Zymosan is a nonbacterial, nonendotoxic agent that produces liver, intestine, lung, and kidney failure in test animals. In mice treated with 40 mg/kg of GPI 18214, which has an IC₅₀ value of 3.0 μM, all signs of pancreatic, renal, and liver damage were abolished, and lung injury was significantly reduced after induction of zymosan.⁶⁸ The protective effects of GPI 18214 correlate with reduced neutrophil and PMN lysosome infiltration into the intestine and lung, which have been known to augment tissue damage through inflammatory effects.^{69,70} It is hypothesized that these PARG inhibitors might not only reduce infarct volumes by stopping the vicious cycle of NAD⁺ depletion, but also by their anti-inflammatory effects, as described below.^{29,32,68}

Studies with tannins. The in vivo effects of tannins have been thoroughly studied. While the more potent PARG inhibitors nobotanin E (trimer) and K (tetramer) have little inhibitory effect on the catabolism of PAR in intact 34I cells, it has been found that nobotanin B causes concentration-dependent inhibition of PAR catabolism.⁶⁵ The inability of nobotanin E and K to inhibit PARG is due to the poor penetration of these compounds into plasma membranes. When cell extracts incubated with various tannins were analyzed by HPLC, it was found that only nobotanin B could be taken up by the cells in a significant level,⁶⁵ thus suggesting that a major drawback of these compounds are their high molecular weight.

PARG enzymatic activity also appears to directly aid the process of DNA damage repair.⁷¹ Using the specific competitive inhibitor Oen B, the role of PAR catabolism by PARG in synchronized HeLa S3 cells at G1 phase has been studied. Due to the fact that the process of excision repair synthesis of DNA requires large amounts of energy, it has been hypothesized that the energy produced during PAR catabolism by PARG is directly involved in this DNA repair. In fact, when ³H-labeled thymidine 5′-triphosphate (3H-dTTP) was added to poly(ADP-ribosyl)ated nuclei from G1-sychronized HeLa cells that were subsequently treated with the DNA-damaging agent MNNG, ³H-dTMP incorporation into the DNA began after a short 3-minute lag.⁵⁸ On the other hand, in the presence of Oen B very little incorporation of ³H-dTMP was observed, suggesting that energy in the form of ATP produced by PAR catabolism serves as a direct energy source for repair synthesis. Furthermore, in cases of moderate DNA damage, such as that caused by NO donors such as spermine nonoate (SNO), addition of the PARG inhibitor gallotannin greatly decreases the ability of rat germinal cells to recover.⁷² In this case, the important role of PARG in protection from DNA damage might be twofold. First, PARP-1 seeks out and repairs DNA strand breaks while suppressing transcription to prevent the expression of damaged genes.^{73,74} If PARP-1 is inhibited by its own auto-poly(ADP-ribosyl)ation, PARG works to restore PARP to its original active status; in short, PARG inhibition ultimately prevents PARP's DNA repair mechanism from functioning properly. Additionally, as explained above, by metabolizing PAR into ADP-ribose monomers and thus adding to the NAD⁺ energy stores, PARG possibly supplies energy needed for the DNA ligation step in base excision repair.⁷¹

On the other hand, in cases of severe DNA damage, PARG inhibition can reduce the amount of cell death observed. A significant decrease in H₂O₂-induced cell death is observed when cultured murine astrocytes are preincubated with as little as 100 nM gallotannin.⁷⁸ Amazingly, gallotannin is 10-fold more potent than the PARP inhibitor benzamide in preventing oxidative cell death. Later studies have shown that nobotanin B, a PARG inhibitor isolated from the plant Tibouchina semidecandra, also offers neuroprotection against both oxidative (induced by both H₂O₂ and N-methyl-D-aspartate) and excitotoxic (induced by MNNG) cell death.¹⁷ Attempts to discover the mechanism of tannin cytoprotection led to the discovery that while gallotannin increases the amount of PAR accumulation in astrocyte cultures, the known PARP inhibitor benzamide actually decreases protein poly(ADP-ribosyl)ation.¹⁷ This indicates that PARG inhibitors do not necessarily inhibit PARP-1 directly but instead slow the turnover of PAR and therefore stop the vicious cycle of NAD⁺ consumption that ultimately leads to cell death through necrosis. While the above explanation of PARG inhibitors involving prevention of an energy crisis in the cell is conventionally accepted, other mechanisms could possibly contribute to the known protective effects of PARG inhibition in cases of severe DNA damage. Since PARP-1 is inhibited by its own automodification by PAR,^{76,77} PARG inhibitors could work to indirectly knock out PARP by preventing catabolism of the inhibitory polymers. Another enzyme, Ca²⁺/Mg²⁺-dependent endonuclease, which is responsible for DNA fragmentation, is also inhibited by poly(ADP-ribosyl)ation and could be indirectly inhibited by PARG in a similar fashion.^{79,80} Although the exact mechanism of action is not totally clear, PARG inhibition by tannin-related drugs has potential as a neuroprotective tool.

In vivo it has been found that PAR metabolism, which is controlled by the concerted action of PARP and PARG, plays a role in the transfer of metabolites, proteins, and drugs across cellular membranes through ATP-binding cassette (ABC) transporters.⁸¹ UVB irradiation causes oxidative stress in cells due to the generation of peroxynitrite (PN) and inhibits the activity of ABC transporters.⁸² Application of either 3,4-dihydro-5(4-(1-piperindinyl)butoxy)-1(2H)-isoquinoline (DPQ), a PARP inhibitor, or the known PARG inhibitor gallotannin restores function of ABC transporters following irradiation, most likely because ADP-ribose itself actually serves as an ABC transport inhibitor.⁸¹ Multiple-drug resistance of tumor cells has often been linked to activity of ABC transporters⁸³, and this discovery highlights yet another potential application of PARG to cancer therapy.

Remarkably, PARG has also been linked to the regulation of transcription and consequently the expression of proinflammatory genes. When a PARG inhibitor such as gallotannin is incubated with macrophages, PAR accumulates, which in turn causes expression of inducible nitric-oxide synthase (iNOS) and cyclooxygenase-2 (COX-2), but not interleukin-1β or tumor necrosis factor-α.³² This discovery is further supported by the fact that silencing of PARG with small interfering RNA (siRNA) prevents gallotannin-mediated expression of iNOS and COX-2.³² At this point, it is necessary to mention that chromatin condensation, which is one of the markers of gene down-regulation, has been shown to be tied to PARG and its associated PAR catabolism.⁸⁴ Conversely, inhibition of PARG and the consequent buildup of PAR is associated with chromatin loosening,⁸⁵ which allows for gene transcription. Taken together, these data indicate that PARG inhibition can increase immune cell activation and therefore have major impacts on inflammation.

Through the use of the tannins as putatively selective inhibitors of PARG, the neuroprotective effects of PARG inhibition have been demonstrated. However, recently it has been suggested that while gallotannin may act as a PARG inhibitor, this compound is promiscuous and ineffective in vivo.⁸⁶ Initial questions concerning the specificity of gallotannin for PARG first arose upon close examination of its properties as provided by its manufacturer, namely that gallotannin is known to form insoluble complexes with proteins and acts as a fixative/stain. In models of H₂O₂- and MNNG-mediated cytotoxicity, it was found that while gallotannin prevents cell death in oxidative models as expected, in the studies involving nonoxidative DNA damage this polyphenol has no effect. Additionally, two other polyphenols, quercetin and catechin, were shown to alleviate cell death in the same model at concentrations similar to gallotannin, although they have never been reported as PARG inhibitors. One would also expect that PARG inhibition by gallotannin in living cells would enhance PAR production, but it has actually been found to reduce PAR formation, an effect attributed to its reactive oxygen species scavenging ability.⁸⁶ The strong H₂O₂-scavenging properties of tannins at low concentrations have been well documented,⁸⁷ and the major component of gallotannin, tetra-galloyl-glucose, contains 13 phenolic hydroxyl groups, and 12 are in ortho positions and primed to react with hydroxyl radicals. While gallotannin does act as a PARG inhibitor in cell-free assays, it also has antioxidant properties and can protect cells from oxidative stress without PARG inhibition. Therefore, because gallotannin is not ideal for the evaluation of PARG in cellular death models, all results obtained previously using this compound must be carefully reviewed.⁸⁶

Studies with substrate analogues. Along with an array of microtubules and their associated proteins, which act to align and separate chromosomes, it has been found that PAR is a nonproteinaceous, nonchromosomal component of the spindle necessary for bipolar spindle assembly and function.¹³ Enriched at both the spindle poles and kinetochores, cellular PAR concentration markedly increases during metaphase and anaphase-telophase due to heightened PARP activity,⁸⁸ which indicates that PARG itself might be important in regulating spindle disassembly/assembly as well. After addition of 100 μg/ml of PARG to Xenopus egg extract spindles, a rapid breakdown of spindle structure is observed. Due to this hydrolytic activity of PARG, which can indeed be inhibited by the addition of ADP-HPD, microtubules were observed to turn outward from the spindle center, causing disconnection of the two half-spindles. Along with PARP and PAR, PARG is found to co-localize with spindle microtubules in BSC1 and HeLa cells, once again indicating that it actively works to regulate PAR levels in the spindle.

As highlighted above, three major classes of PARG inhibitors exist to date: ADP-ribose analogues, DNA intercalators, and tannins. While the last two classes of compounds show considerable potency, neither are ideal because of their high toxicity, high molecular weight, and inability to act as specific inhibitors. Due to their highly charged nature, ADP-ribose analogues are not cell-permeable and are therefore unsuitable for use as inhibitors in a biological context. Additionally, such substrate analogues closely resemble biologically relevant molecules, which may ultimately be a source of unwanted side effects. Despite the lack of optimal inhibitors, it is clear that PARG is an attractive target for drug discovery and development. In consideration of the aforementioned problems, other classes of novel and potent PARG inhibitors are currently being sought. Armed with new methods to rapidly screen for PARG inhibition (see Putt & Hergenrother (2004); Putt et al. (2005) and other examples herein), combinatorial libraries and/or small molecule collections can be screened to locate a novel potent inhibitor of PARG. As new and selective inhibitors of PARG are identified and developed, these inhibitors can be used to elucidate and define new and exciting roles for cellular poly(ADP-ribosyl)ation.

### EXAMPLE 2

### Identification of PARG Inhibitors

As mentioned herein, there is considerable evidence indicating small molecule inhibitors of PARG are likely effective neuroprotection agents in Parkinson's Disease (PD). Unfortunately, to date no adequate cell-permeable PARG inhibitors have been identified, due in large part to the lack of suitable enzyme assays and screens. Significantly, we have recently developed a high-throughput screen for the identification of PARG inhibitors.⁵⁹ We have now obtained potent, cell permeable small molecule inhibitors of PARG, and have demonstrated that these potent PARG inhibitors are neuroprotective in cell culture models of PD.

Poly(ADP-ribosyl)ation and its relevance to Parkinson's Disease. Poly(ADP-ribosyl)ation, PARP, and PARG. In response to DNA damage, the enzyme poly(ADP-ribose) polymerase (PARP) catalyzes the addition of ADP-ribose (through NAD⁺) units to glutamic acid residues of substrate proteins.^{1,90} (FIG. 5) The poly(ADP-ribose) (PAR) polymer produced can be well over 100 monomer units long and often contains multiple sites of branching. Although PARP-1 accounts for the majority of cellular PARP activity, there are at least 17 predicted PARP isozymes, and 8 have been identified to date.²⁴ One of the major substrates for PARP-1 is the PARP-1 protein itself, and this automodification ultimately leads to the inactivation of the enzyme through electrostatic repulsion between the PAR polymer and the damaged DNA. In contrast to the multiple PARP isozymes, the cell uses a single enzyme, poly(ADP-ribose) glycohydrolase (PARG) to catabolize PAR to ADP-ribose.³ This “PARP cycle” is depicted in FIG. 2A. As a consequence of this cycle, inhibition of PARG results in the inhibition of PARP—PARG inhibition locks PARP in the auto-modified, inactive state.

PARP-1 is activated in MPTP-induced cell death. Animal models based on the toxicity of 1-methyl-4-phenyl-1,2,3,6-tetrahydropyridine (MPTP) replicate many of the features of PD, including selective death of neurons in the substantia nigra pars compacta, α-synuclein aggregations, and severe Parkinsonian symptoms indistinguishable from sporadic PD. This is largely accepted to be the most accurate model of sporadic PD.⁹¹ The lipophilic MPTP quickly passes through the blood-brain-barrier and is oxidized to MPP⁺, which localizes to dopaminergic neurons through its affinity for dopamine transporters. Once inside these neurons, MPP⁺ builds up in the mitochondria and inhibits complex I of the electron transport chain, setting off a cascade of events that ultimately results in the generation of substantial amounts of reactive oxygen species (ROS). ROS cause DNA damage, which in turn induces PARP-1 activity. This PARP-1 activity, in addition to exacerbating the already-reduced levels of ATP, leads to the translocation of Apoptosis Inducing Factor (AIF) from the mitochondria to the nucleus, resulting in DNA fragmentation and cell death.^{92,93} This connection between MPTP-mediated cell death and PARP activity was profoundly demonstrated with PARP-1^{(-/-)} mice—mice lacking the PARP-1 gene are almost totally saved from MPTP neurotoxicity.⁹⁴

PARG inhibitors as potential neuroprotective agents in PD. A variety of PARP inhibitors have been described,^{27,95} and studies with these compounds have shown their administration will protect animals from MPTP toxicity and other neurotoxins.⁹⁶⁻⁹⁹ However, it is widely recognized that these inhibitors are not ideal due to the multiple number of PARP isozymes (no isozyme-specific PARP inhibitors have been identified to date) and the fact that virtually all of these inhibitors bind to the NAD⁺ binding site of the PARPs. Both of these traits lead to multiple non-specific off-target effects; additionally these compounds have poor bioavailability in the central nervous system.⁹⁴ Because inhibition of PARG results in the inhibition of PARP (see FIGS. 2-3), PARG inhibitors have great potential as neuroprotective agents for PD treatment. With only one isozyme, off-target effects of appropriate PARG inhibitors would be minimal. Unfortunately, there are no reported potent, specific, cell-permeable inhibitors of PARG.¹⁰⁰

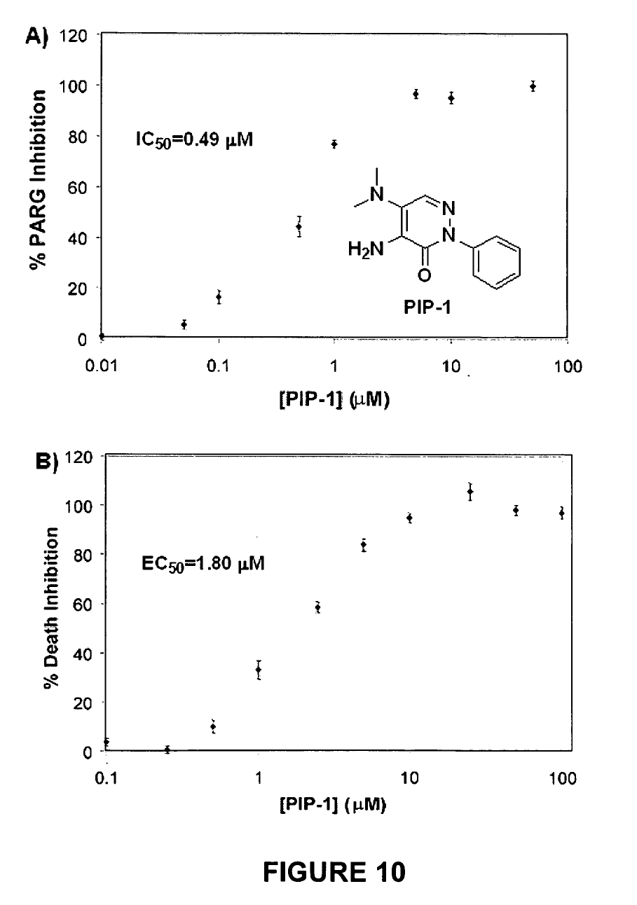

Identification of a potent, cell-permeable PARG inhibitor by screening compound collections and subsequent cell culture studies. The Hergenrother laboratory owns a collection of ˜22,000 small molecules, each formatted as 10 mM stock solutions in DMSO in 384-well plates. Approximately 8,000 of these compounds were synthesized or collected. Recently, we screened this collection using a modified variant of our high-throughput PARG assay as discussed herein. We have identified three highly potent small molecule PARG inhibitors. The structure of the most potent of these compounds, which we have named PIP-1 (Pyridazine Inhibitor of PARG), is shown in Table 2 (structure labeled PIP-1, and structure 23, synthesized PIP-1). PIP-1 inhibits PARG in vitro with an IC₅₀ of 0.49 μM (FIG. 10A), and shows excellent protection of differentiated PC-12 and SK-N-SH cells from MPP⁺ toxicity, with an IC₅₀ of ˜1.8 μM in these cell culture assays (FIG. 10B). This level of protection at micromolar PIP-1 concentrations is quite remarkable; for instance, a recent study showed that 1 mM concentrations of talipexole are required for protection in the MPP⁺ human neuroblastoma cell culture model.¹⁰¹ Thus PIP-1 is not only a promising drug candidate, but the protection observed with this compound is fully consistent with the notion that PARG inhibition can provide neuroprotection in PD. Importantly, this compound also appears to be completely non-toxic, having no adverse effects on the differentiated neurons at concentrations as high as 100 μM. Finally, we show that PIP-1, through its PARG inhibition causes the inhibition of PARP-1 activity in cell culture.

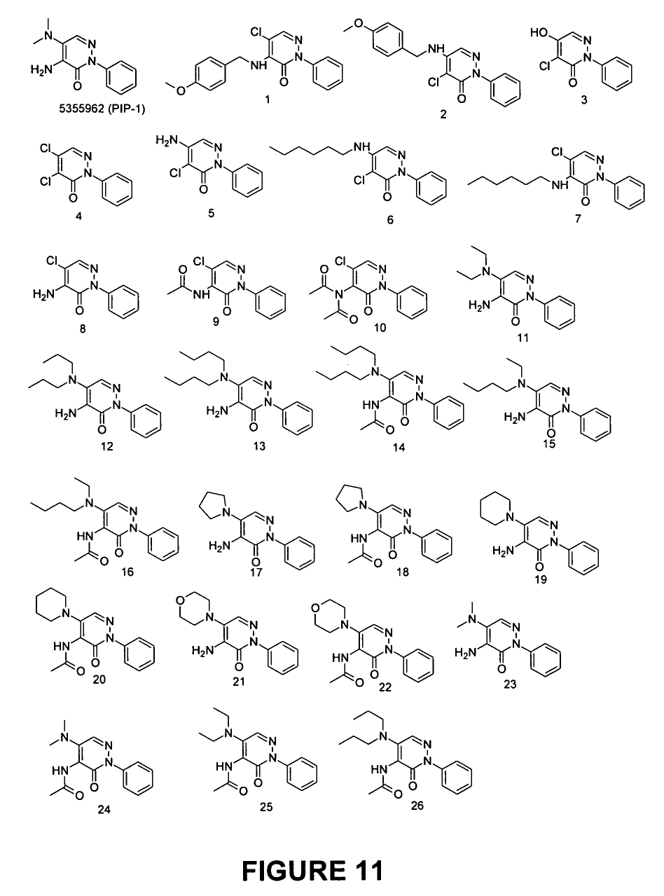

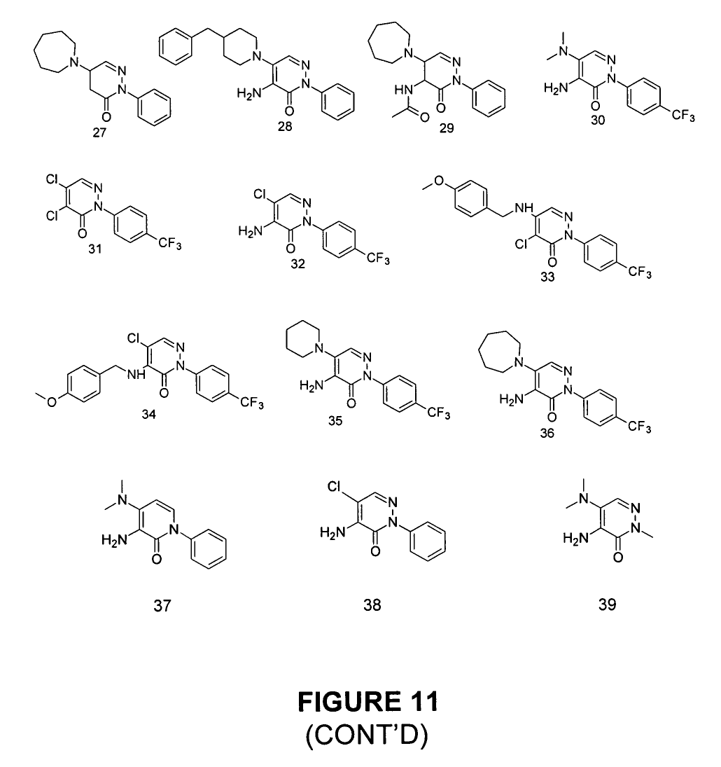

We have made multiple derivatives of PIP-1; these compounds are shown below in Table 2 and in FIG. 11 and labeled Structures 1-39. The neuroprotective effects of structures 1-26 were tested in the cellular assay and compared to the neuroprotective effect of PIP-1 (FIG. 13). As shown in FIG. 13, structures 11, 12, 13, 19, 20, 21, 23 have activity similar or better than PIP-1. Structures 2, 15, 17, 18, 22, 24, had moderate activity. Structures 1, 3, 4-10, 14, 16, 26 showed low or no activity in this assay:

### EXAMPLE 3

### Method of Making 4-Amino-5-(Disubstituted Amino)-3(2H)Pyridazinones

Scheme 1 illustrates one possible synthesis route for 4-Amino-5-(Disubstituted amino)-3(2H)Pyridazinones and TABLE 1 various R groups.

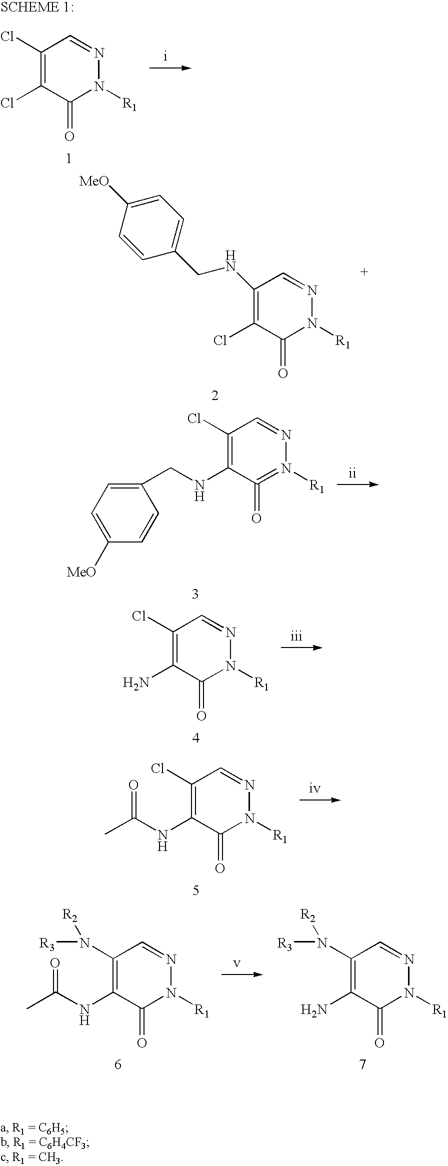

TABLE 1
Representative R₂ and R₃ Groups for Compounds 6 and 7

| 6, 7 of Scheme 1 | R₂ | R₃ |
|---|---|---|
| 1 | CH₃ | CH₃ |
| b | C₂H₅ | C₂H₅ |
| c | C₃H₇ | C₃H₇ |
| d | C₄H₉ | C₄H₉ |
| e | C₂H₅ | C₄H₉ |

| | |
|---|---|
| f* | —C₄H₈— |
| g* | —C₅H₁₀— |
| h* | —C₆H₁₀— |
| i* | —(CH₂)₂O(CH₂)₂— |
| j* | 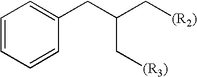 |

*Lettered substituent groups f, g, h, I, and J indicate R₂ and R₃ groups taken together. For example, substituent J explicitly denotes the connection points for R₂ and R₃ which together ultimately form a ring with the N atom of compounds 6 and/or 7 of Scheme 1.

The starting material 4,5-Dichloro-2-phenyl-3(2H) pyridazinone (1a) was purchased from Aldrich. 4,5-Dichloro-2-(4-trifluoromethyl)phenyl-3(2H) pyridazinone (1b); 4,5-Dichloro-2-methyl-3(2H) pyridazinone (1c) were prepared by reactions of mucochloric acid with the respective hydrazine in dilute hydrochloric acid.

4-Methoxylbenzylamino-5-chloro-3(2H)pyridazinone (3): A 10 mL vial was charged with 4,5-dichloro-3(2H)pyridazinones 1 (10 mmol), 4-methoxylbenzylamine (2.1 g, 15 mmol), Pd(OAc)₂ (1% mol), BINAP (2% mol), and potassium tert-butoxide (15 mmol). Anhydrous toluene (5 mL) was added into the mixture, and the vial was then sealed with a cap. The reaction mixture was stirred at 140° C. under microwave irradiation (300 W, 250 PSI) for 5 minutes. After the starting 4,5-dichloro-3(2H)pyridazinones 1 was consumed, as monitored by TLC, it was found that two compounds were formed which were separated by eluting with hexane/ethyl acetate, the first fraction was product 3 (yield 76%), the second fraction was product 2 (yield 24%).

4-Amino-5-chloro-3(2H)pyridazinone (4): In a 100 mL round flask, compound 3 (100 mmol) was suspension in 10% HCl (50 mL), the mixture was heated at 100° C. for 6 h, the mixture was cooled down, extracted with CH₂Cl₂ (3×10 mL), the organic layer was combined together, dried with MgSO₄, the product was purified by silica gel with hexane/ethyl acetate (3:1) as elute to give product 4 (yield 95%).

4-Acetylamino-5-chloro-3(2H)pyridazinone (5): To acetyl chloride (50 mL) in a round flask, 4-amino-5-chloro-3(2H)pyridazinone (4) (100 mmol) was added and the reaction mixture was stirred under reflux 3 h. After the reaction mixture was cooled down, evaporated acetyl chloride, the residue was purified by silica gel using hexane/ethyl acetate (1:1) to yield compound 5 (97%).

4-Acetylamino-5-(disubstituted amino)-3(2H)pyridazinone (6): A 10 mL vial was charged with 4-Acetylamino-5-chloro-3(2H)pyridazinone 5 (10 mmol), an appropriate secondary or cyclic amine (15 mmol). Anhydrous THF or pyridine (5 mL) was added into the mixture, and the vial was then sealed with a cap. The reaction mixture was stirred at 140° C. under microwave irradiation (300 W, 250 PSI) for 5 minutes. The solvent was evaporated and purified by silica gel, the products were isolated by eluting with CH₂Cl₂/MeOH (20:1) to give products 6 (yield 98%).

4-Amino-5-(disubstituted amino)-3(2H)pyridazinone (7): To a solution of 4-Acetylamino-5-(disubstituted amino)-3(2H)pyridazinone 6 (10 mmol) in ethanol, sodium ethanolate (5 mmol) was added and the reaction mixture was refluxed for 6 h. After the starting material was consumed, ethanol was evaporated, CH₂Cl₂ (20 mL) was added, and washed with H₂O (3×10 mL), the product was purified by silica gel using hexane/ethyl acetate (3:1) to give 7 (yield 96%).

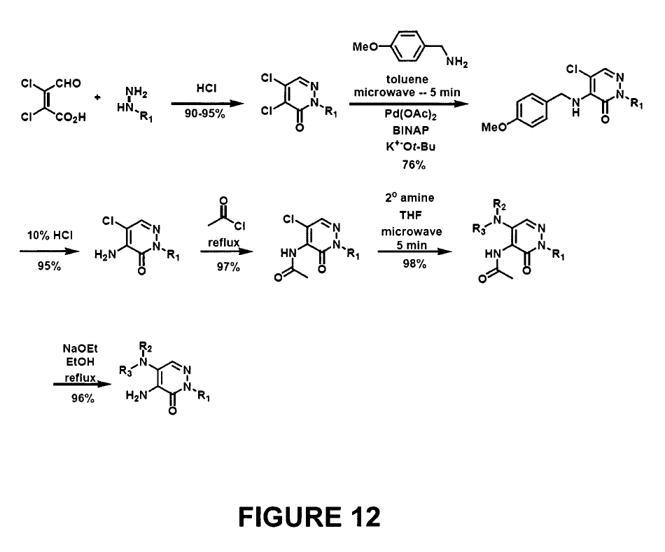

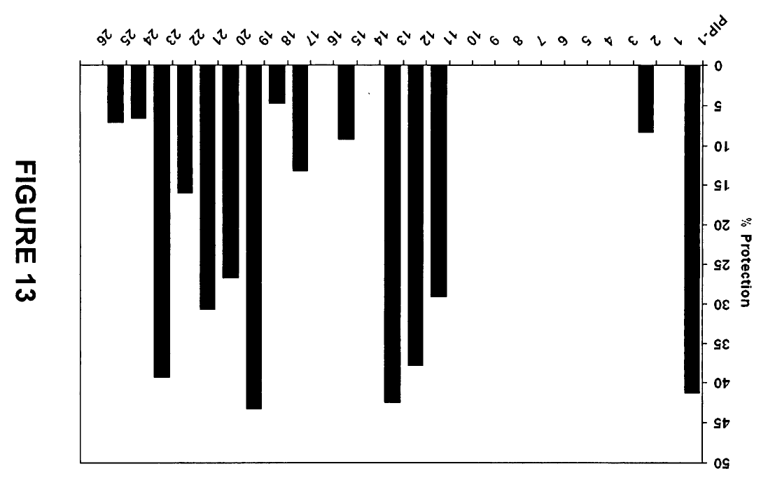

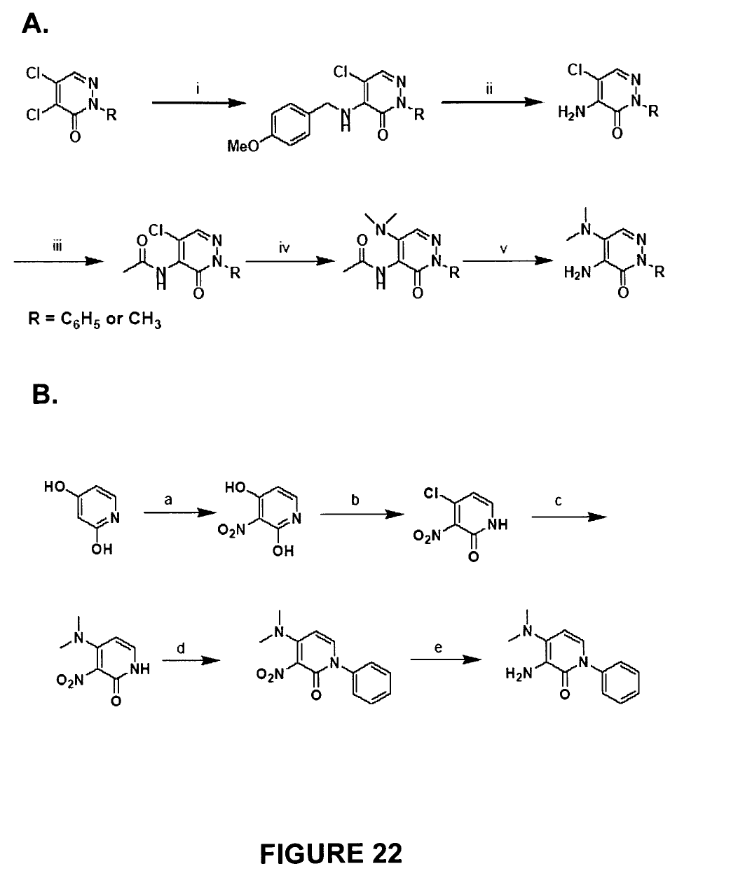

Rapid synthesis of derivatives of PIP-1. FIGS. 12 and 22 further summarize schemes for rapid synthesis of derivatives of PIP-1. Using this scheme, any number of derivatives of the base structure (e.g. PIP-1) is made. More than 100 compounds were made employing one or more of these schemes.

Further variations are prepared according to methods known in the art. In addition, it is understood in the art that alternative schemes can be employed in synthesizing the structures disclosed in the present application.

Representative Structures

TABLE 2
Representative structures of small molecules tested for PARG
inhibition activity (relative to PIP-1).

| Structure Number | Structure | Activity |
|---|---|---|
| PIP-1(see also 23) | 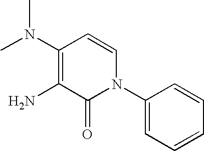 | good |
| 1 | 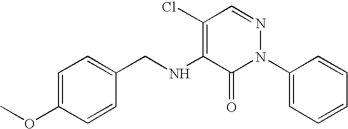 | moderate |
| 2 | 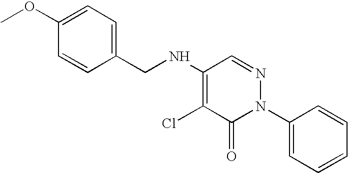 | low |
| 3 | 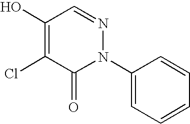 | low |
| 4 | 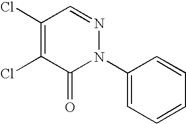 | low |
| 5 | 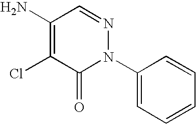 | low |
| 6 | 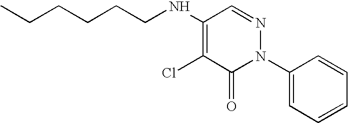 | low |
| 7 | 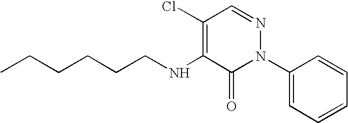 | low |
| 8 | 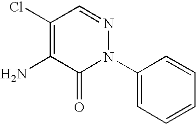 | low |
| 9 | 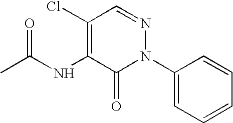 | low |
| 10 | 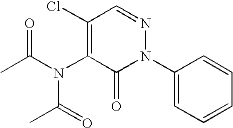 | low |
| 11 | 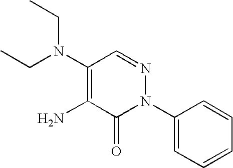 | good |
| 12 | 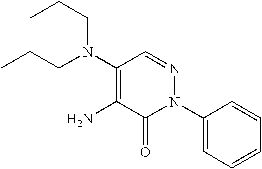 | good |
| 13 | 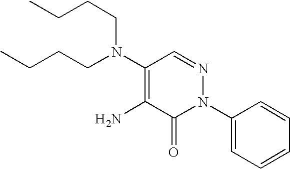 | good |
| 14 | 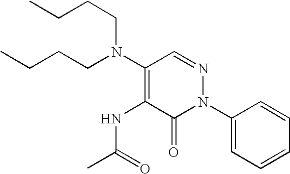 | low |
| 15 | 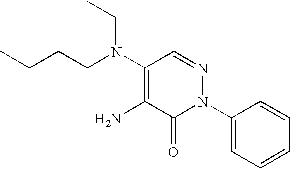 | moderate |
| 16 | 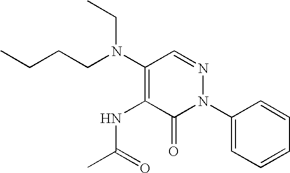 | low |
| 17 | 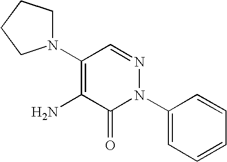 | moderate |
| 18 | 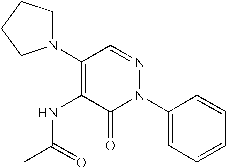 | moderate |
| 19 | 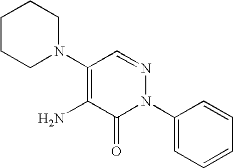 | good |
| 20 | 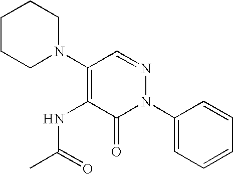 | good |
| 21 | 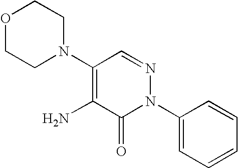 | good |
| 22 | 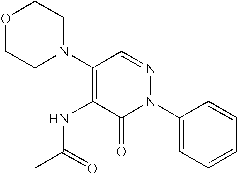 | moderate |
| 23 | 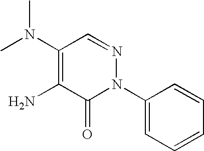 | good |
| 24 | 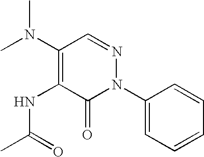 | moderate |
| 25 | 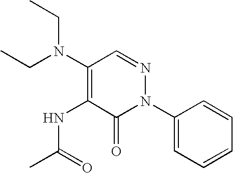 | moderate |
| 26 | 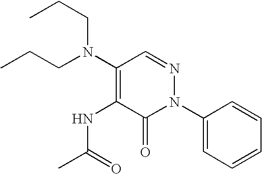 | low |
| 27 | 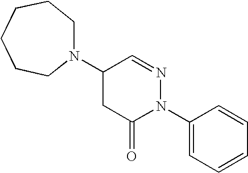 | |
| 28 | 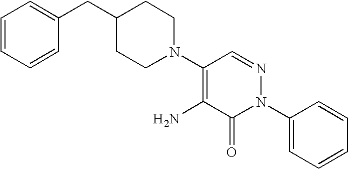 | |
| 29 | 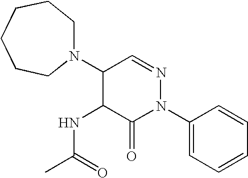 | |
| 30 | 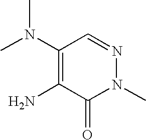 | |
| 31 | 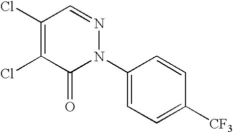 | |
| 32 | 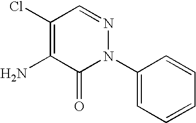 | |
| 33 | 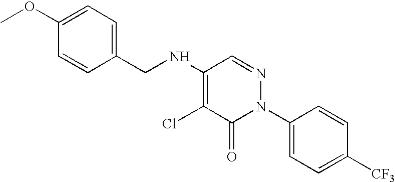 | |
| 34 | 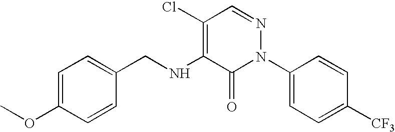 | |
| 35 | 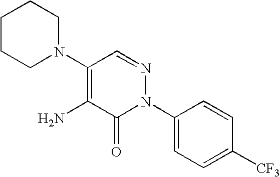 | |
| 36 | 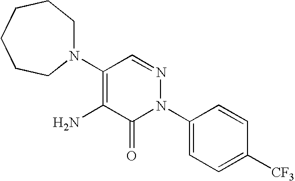 | |
| 37 | 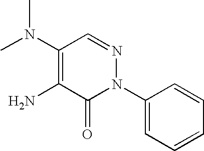 | good |
| 38 | 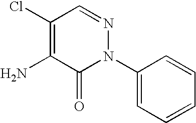 | low |
| 39 | 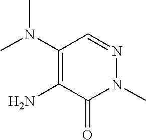 | low |

PIP-1 and structures 1-26 were screened for protection against H₂O₂ treatment of U-937 cells FIG. 13. In this initial evaluation for neuroprotection, compounds 11, 12, 13, 19, 20, 21, and 23 had activity similar or better than PIP-1. Compounds 2, 15, 17, 18, 22, 24, 25 had moderate activity in this assay. Compounds 1, 3-10, 14, 16, and 26 had low or no activity in this assay.

### EXAMPLE 4

### High-Throughput Screen for PARP/PARG Inhibition

Disclosed herein are methods for quantifying PARP activity as a means to distinguish necrotic and apoptotic death in cell and tissue samples and rapid screen for PARP and/or PARG inhibitors. Putt & Hergenrother (2004) disclose a fluorescent method to quantify PARP activity and can be utilized in concert with other techniques disclosed herein as a basis for a high-throughput screen for PARP inhibitors. The screening assay can be effective in whole animal tissue and can be useful as a research or clinical diagnostic tool.

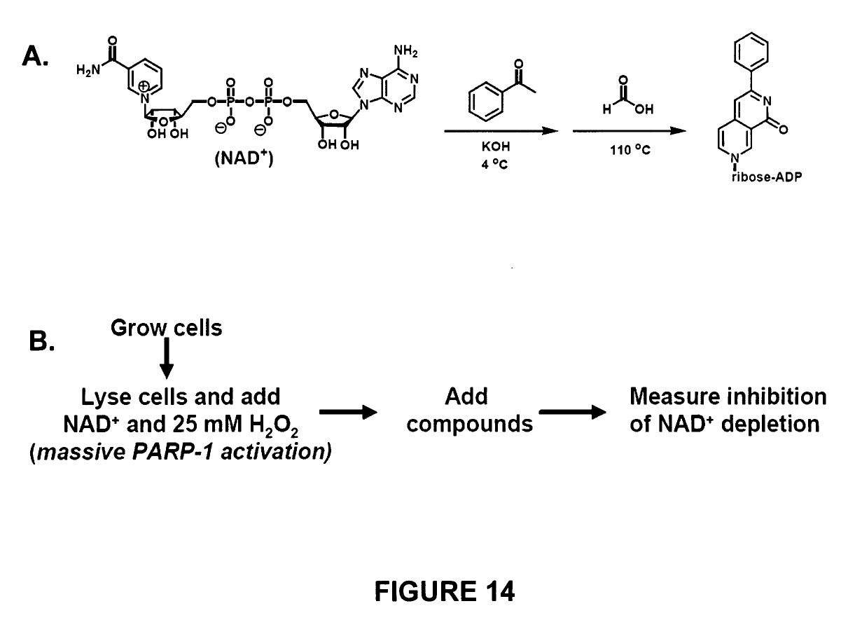

PARP-1 uses NAD⁺ to make poly(ADP-ribose) (FIG. 5). Accordingly, PARP-1 and PARG inhibitors prevent NAD⁺ depletion. Assays disclosed herein identify compounds as possible inhibitors of PARP and/or PARG by measuring NAD⁺ levels in a cell-based system. NAD⁺ levels can be quantified in vitro and in cell-based assays, as summarized in FIG. 14A (see also Putt and Hergenrother, Anal. Biochem 326: 78 (2004) and Putt et al., ChemBioChem 6:53 (2005)). Briefly, the sample is incubated for about 10 minutes at 4° C. in a KOH and acetophenone solution. Formic acid is then added and the sample incubated for 5 min. at 110° C. The sample is then cooled and fluorescence measured at an emission wavelength of about 444 nm with an excitation wavelength of about 372 nm.

The high-throughput screen for PARP and PARG inhibitors can employ a method summarized by the flow diagram of FIG. 14B. A cell line is grown and lysed, to which NAD⁺ and H₂O₂ are added. Any activator of PARP or PARG can be used. H₂O₂ is a known activator of PARP-1 and 25 mM H₂O₂ causes massive PARP-1 activation. Accordingly, NAD⁺ levels are depleted. The compound to be screened is added to the solution and levels of NAD⁺ measured, including fluorometically measured, as summarized in FIG. 14A. A compound is identified as a potential PARG inhibitor if there is an inhibition of NAD⁺ depletion. This screen is used to screen about 22,000 compounds, with 38 hits, as discussed in the next example.

The methods and screens disclosed herein are further adapted so as to provide a screen for PARG inhibitors. The screening assay is effective in whole animal tissue and is useful as a research or clinical diagnostic tool.

### EXAMPLE 5

### Protection by a Small Molecule PARG Inhibitor in Models of Parkinson's Disease

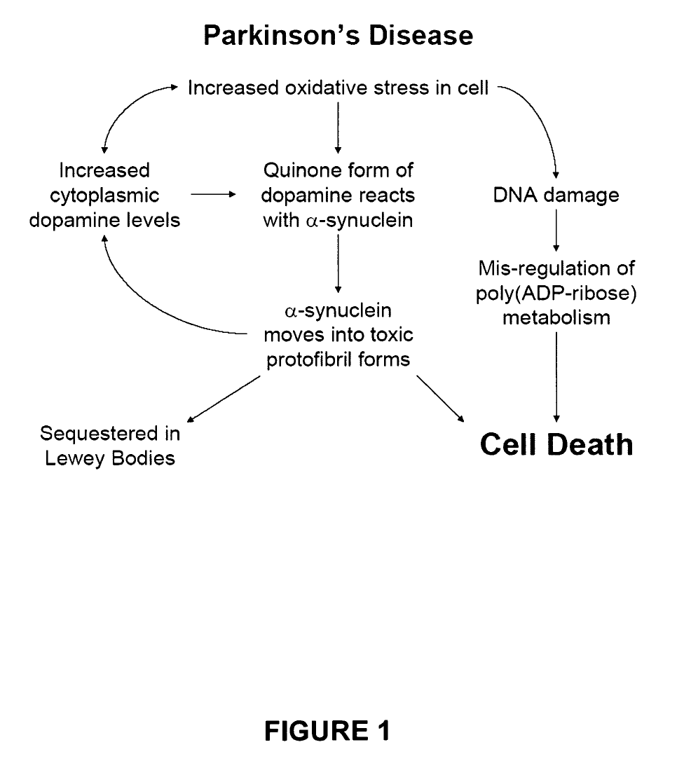

Parkinson's disease (PD) is a debilitating neurodegenerative disorder afflicting 1-2% of the over-65 population. FIG. 1 provides a summary of the mechanisms of PD believed to play a role in neurodegeneration. No available drugs have been shown to be neuroprotective in PD patients, and none slow the progression of this disease. Previous work shows that mice lacking the gene for poly(ADP-ribose) polymerase-1 (PARP-1) are resistant to the neurotoxic effects of 1-methyl-4-phenyl-1,2,3,6-tetrahydropyridine (MPTP), a compound known to induce a Parkinson's-like state in mice, non-human primates, and humans. Unfortunately, small molecule inhibitors of PARP-1 are problematic for the treatment of PD due to their lack of specificity and lack of bioavailability in the brain. This example shows that the inhibition of poly(ADP-ribose) glycohydrolase (PARG) provides protection in cell culture and animal models of PD. Inhibition of PARG is known to lock PARP-1 in a poly(ADP-ribosyl)ated, inactive state, thus PARG inhibitors indirectly inhibit PARP-1. As no satisfactory PARG inhibitors were available, we first identified a potent PARG inhibitor (called PIP-1) through a high-throughput screen. PIP-1 induces the buildup of poly(ADP-ribose) in cell culture, and provides protection of differentiated neuronal cell lines from the common neurotoxins rotenone and MPP+. PIP-1 also protects primary dopaminergic neurons from rotenone, and derivatives of PIP-1 that do not inhibit PARG show no protection in cell culture. Finally, PIP-1 penetrates the blood-brain-barrier and protects mice from the toxic effects of MPTP. The combined data indicate that inhibition of PARG, including by PIP-1 and derivatives thereof, is a viable therapeutic strategy for the treatment of PD.

PD is the second most common neurodegenerative disorder, affecting approximately 1-2% of the population over the age of 65 and over 1 million people in the United States alone. The characteristic symptoms of PD include resting tremor, bradykinesia, and postural instability, while the two major pathological hallmarks are the death of dopaminergic neurons in the substantia nigra pars compacta, and the formation of intracellular inclusions know as Lewey bodies, which primarily consist of fibrillar α-synuclein (see FIG. 1). There is currently no cure for PD, and no therapies even slow the progression of the disease. Some symptoms of PD can be temporarily treated with dopamine replacement therapy (through administration of L-dopa), dopamine agonists (pramipexole, ropinirole), or inhibition of dopamine metabolism (entacapone, selegiline), but all of these treatment regimens lose their effectiveness over time. (1, 2)

Although the clinical manifestations of PD typically arise after significant dopaminergic neuronal cell death has taken place, advancing technologies for early PD detection have made viable therapeutic strategies based on neuroprotection. Unfortunately, no drugs are available that provide neuroprotection to PD patients (3, 4) despite the recognized need for such treatments. (5) In both familial and sporadic PD, mitochondrial dysfunction appears to be at the heart of the aberrant neuronal cell death. (6) In particular, considerable evidence has implicated the inhibition of mitochondrial complex I in the PD brain, which leads to an increase in free radicals, oxidative stress, and ultimately cell death. Complex I poisons such as the pro-toxin 1-methyl-4-phenyl-1,2,3,6-tetrahydropyridine (MPTP) and rotenone have been shown to induce states strongly resembling PD in laboratory animals (7) and humans unknowingly exposed to MPTP present with a Parkinson's-like syndrome (8) In addition, patients with familial PD harbor mutations are believed to lead to mitochondrial dysfunction (6, 9)

It is increasingly recognized that the protein poly(ADP-ribose) polymerase-1 (PARP-1) plays a significant role in the neuronal cell death in PD brains. (10) PARP-1 is activated by DNA damage and catalyzes the synthesis of poly(ADP-ribose) (PAR) polymers from NAD⁺. PARP-1 appends PAR to a variety of substrates, including itself in an auto-modification event; this PAR synthesis leads to DNA damage repair through a variety of mechanisms. The auto-modification of PARP-1 leads to a strong electrostatic repulsion between the poly(ADP-ribosyl)ated PARP-1 and the DNA, and PARP-1 is inactivated by this auto-modification. A second enzyme, poly(ADP-ribose) glycohydrolase (PARG), catalyzes the catabolism of PAR polymers to monomeric ADP-ribose units in an endo- and exo-glycosidic manner. (11) (FIG. 5) This removal of PAR from PARP-1 reactivates PARP-1 by freeing it to once again bind damaged DNA. Thus PARP-1 and PARG form a cycle of poly(ADP-ribosyl)ation/PARP-1 inactivation, PAR catabolism/PARP-1 re-activation;(12) a consequence of this cycle is that inhibition of PARG results in the inhibition of PARP-1, as PARG inhibition locks PARP-1 in the auto-modified, inactive state.

As the oxidative stress observed in PD is believed to induce PARP-1 activity, (13) the role played by PARP-1 in PD has been investigated. Evidence for the involvement of PARP-1 in PD comes from a study showing elevated levels of PARP-1 in dopaminergic neurons of PD patients. (14) In addition, studies with both PARP-1 knockout mice and small molecule inhibitors of PARP-1 have implicated PARP-1 as a key mediator of the MPTP-induced neurotoxicity in mice: PARP-1^{(-/-)} mice are resistant to the effects of MPTP, (15) and pharmacological inhibitors of PARP-1 show protective effects in the MPTP mouse model of PD. (16-18) Further studies have shown that nitric oxide(NO)-induced DNA damage is required for PARP-1 activation and MPTP toxicity, likely through the combination of NO with O²⁻ to form peroxynitrite. (15) This PARP-1 activation results in the depletion of cellular NAD⁺/ATP stores, ultimately leading to cell death.

Unfortunately, small molecule inhibition of PARP-1 for disease treatment is complicated by several factors. First, the PARP family consists of at least 18 isozymes, (19) and to date no compounds have shown any significant isozyme-specific inhibition; that is, compounds that inhibit PARP-1 inhibit the other PARP isozymes to a similar degree. Although the function of the majority of these isozymes is unknown, some of them (e.g., tankyrase (20)) appear to play very important and non-redundant roles in the cell, and their inhibition with a non-specific PARP inhibitor could complicate this therapeutic strategy. Second, the vast majority of these inhibitors bind to the PARP NAD⁺ binding site, (21) increasing the chance for off-target effects with other NAD⁺ binding proteins. Third most of these PARP inhibitors have poor bioavailability in the central nervous system. (22) Finally, direct inhibition of PARP-1 leaves the cell in a low PAR state, one unable to activate the endogenous DNA damage repair system.

The direct inhibition of PARG locks PARP-1 in the inactive state and thus indirectly inhibits PARP-1. PARG inhibition offers advantages for neuroprotection over direct PARP-1 inhibition, namely, there is only one PARG isozyme, and PARG inhibition leaves PAR on the protein substrates, possibly facilitating cellular repair and survival. (11, 23) Unfortunately, typical PARG inhibitors are either large molecules that inhibit the enzyme non-specifically (e.g., tannins), (24, 25) DNA intercalators that are toxic (e.g., ethacridine), (26) or substrate analogues that are not cell permeable (e.g., ADP-HPD). (27) The lack of a potent, specific, cell permeable small molecule inhibitor of PARG has hampered the interrogation of PARG as a therapeutic target. Herein we report the identification of a novel PARG inhibitor and describe its protective effects in cell culture, primary neurons, and in a mouse model of PD.

To identify a non-toxic, non-DNA intercalating, cell permeable small molecule inhibitor of PARG, a high-throughput screen is devised. Given the severe challenges in isolating enough of the PAR substrate for an in vitro screen directly for PARG inhibition, we develop a primary screen that utilizes cell lysates and, in particular, mammalian cell lysates. In this assay, HL-60 cells are treated with H₂O₂ to damage their DNA (which activates PARP-1) and then lyse the cells. In individual wells of a 384-well plate, this cell lysate is incubated with 22,000 different small molecules from an in-house compound collection; these compounds are either purchased (from ChemBridge Corp.) or collected from various sources at the University of Illinois. (28) NAD⁺ levels are measured using a fluorescent method based on the chemical conversion of NAD⁺ to a fluorescent product (29, 30) (FIG. 14A). The amount of NAD⁺ consumed in each well is monitored, and any compounds that prevent NAD⁺ consumption are classified “hits” in this primary screen. Compounds that prevent NAD⁺ consumption in the assay can do so either by inhibition of PARP-1 (which inhibits the conversion of NAD⁺ to the poly(ADP-ribose) product), or through the inhibition of PARG (which locks PARP-1 in its inactive state), or through some other mechanism.

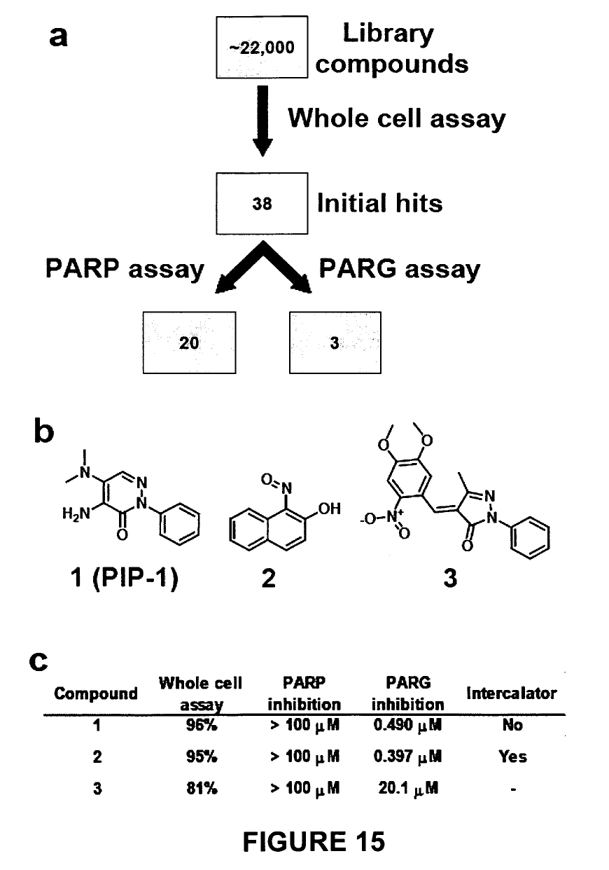

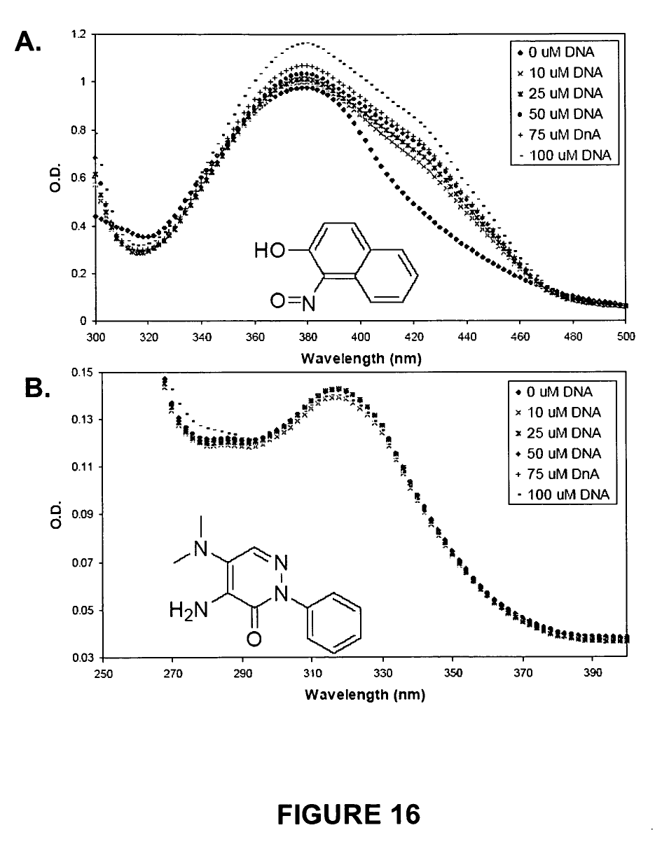

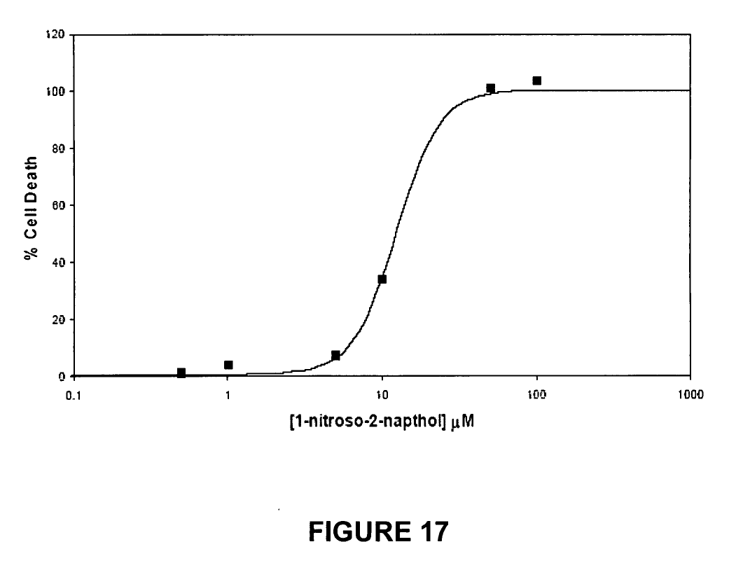

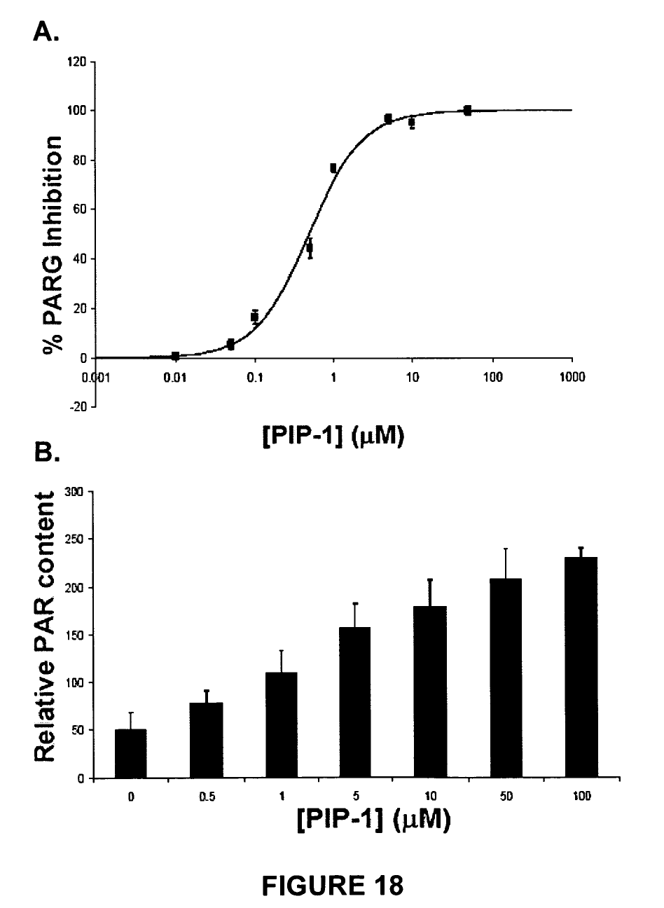

Screening of the 22,000 compounds in this manner produced 38 primary hits (FIG. 15A—“initial hits”). These 38 compounds are assessed for their ability to inhibit PARP-1 and PARG using standard in vitro assays. (12, 29) 20 compounds are PARP-1 inhibitors, and 3 are PARG inhibitors (FIG. 15A). The structures of the three PARG inhibitors are shown in FIG. 15B. Compounds 1 and 2 inhibit PARG quite well, with IC₅₀ values of ˜0.4-0.5 μM (FIG. 15C). Further experimentation with these compounds reveal that while compound 1 does not intercalate into DNA, compound 2 is a DNA intercalator (FIG. 16) and is toxic to cells in culture (FIG. 17), and is thus unattractive as a candidate for in vivo PARG inhibition. Subsequent efforts therefore focused on compound 1, which is named PIP-1 (pyridazinone inhibitor of PARG), and derivatives thereof. PIP-1 inhibits PARG in vitro in a dose-dependent manner (FIG. 1d) with an IC50=0.49 μM. To determine if PIP-1 could induce the build-up of PAR in cell culture, PC-12 cells were treated with a range of PIP-1 concentrations; H2O2 was then added to induce PARP-1 activity and the cellular PAR levels were quantitated with an anti-PAR antibody and flow cytometry. As shown by the graph in FIG. 1e, PIP-1 causes a dose-dependent accumulation of intracellular PAR, presumably through the direct inhibition of PARG, indicating it is a cell permeable inhibitor of PARG.

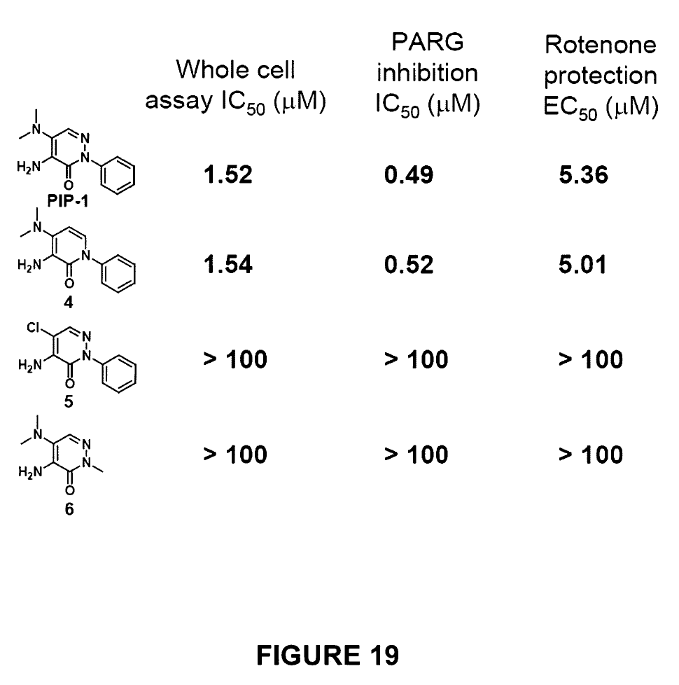

Rotenone is a complex I poison and is widely used in cell culture and animal models of PD. (20, 31, 32) To determine if PIP-1 protects cells from rotenone insult, PC-12 cells are neuronally differentiated (with neuronal growth factor β for 10 days) and then treated with 10 μM rotenone and a range of PIP-1 concentrations. After 72 hours, cell death is assessed using propidium iodide staining as measured by flow cytometry. PIP-1 provides cellular protection in this assay, with an IC₅₀ of 5.36 μM (FIG. 19). To determine the features on PIP-1 that are important for PARG inhibition and cell protection, three derivatives are constructed that lacked key functionality (see FIG. 19 compounds labeled 3, 4 and 5). Deletion of one of the ring nitrogens of the pyridazinone had no effect on activity, as lactam 4 had virtually identical PARG inhibition and cell protection properties as PIP-1. In contrast, a compound lacking the dimethyl amine (compound 5) and one missing the phenyl ring (compound 6) are inactive in these assays (FIG. 19).

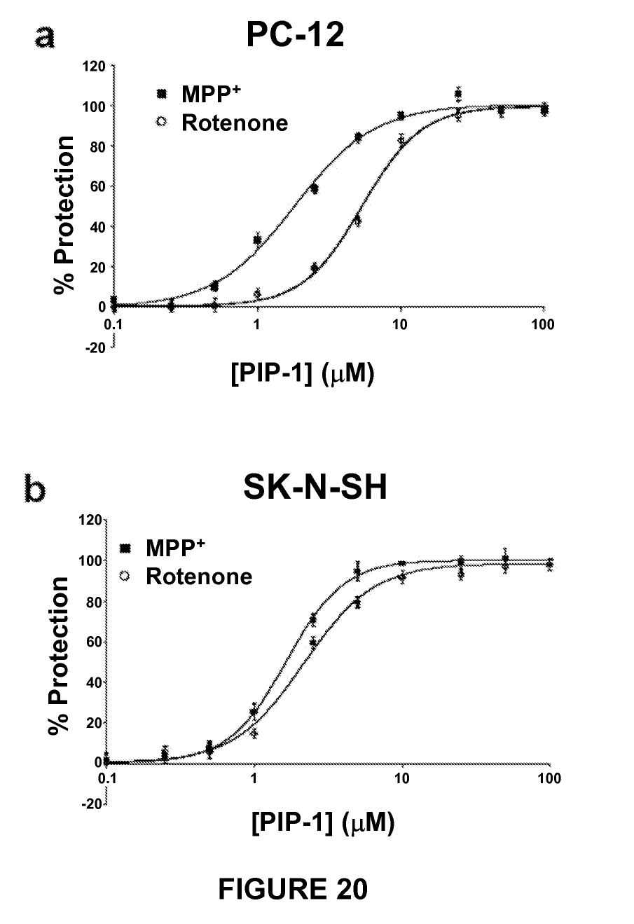

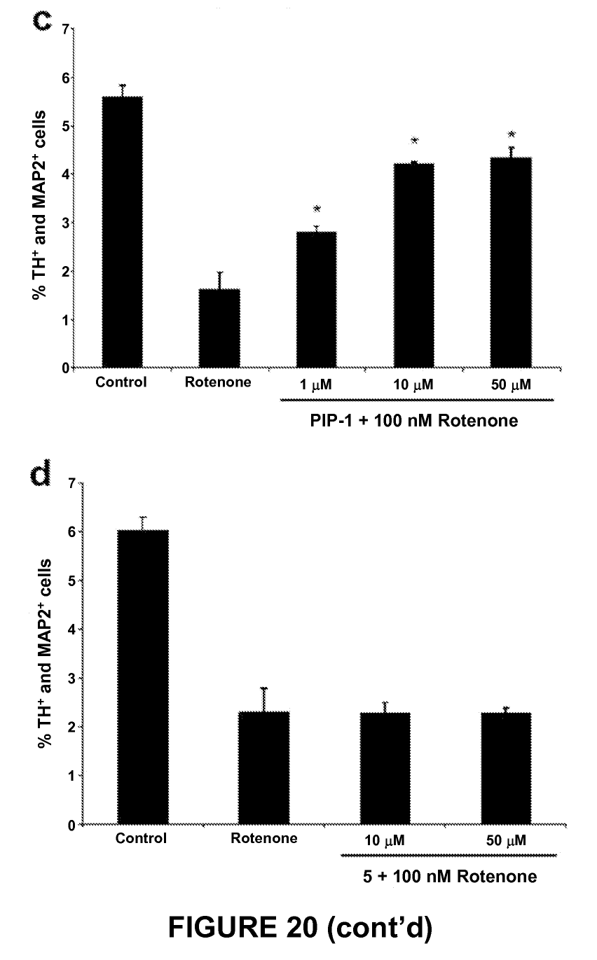

In vivo MPTP is metabolized to its active form, the 1-methyl-4-phenylpyridinium ion (MPP⁺), and MPP⁺ has proven very useful in a variety of cell culture models of PD. To determine the protective effect of PIP-1 on cells subjected to MPP⁺ insult, PC-12 (differentiated as above) and SK-N-SH (differentiated with retinoic acid for 12 days) cells are treated with 10 μM MPP⁺ and a range of PIP-1 concentrations, and cell death is assessed by propidium iodide staining and flow cytometry. As shown by FIGS. 20A and 20B, PIP-1 provides protection from MPP⁺-induced cell death in both of these cell lines, with IC₅₀ values of 1.81 μM and 1.64 μM for PC-12 and SK-N-SH, respectively. As shown in FIGS. 20A and 20B, PIP-1 also protects differentiated PC-12 and SK-N-SH cells from rotenone-induced toxicity as well. Importantly, PIP-1 showed no cellular toxicity in any of these assays at concentrations up to 100 μM.

With in vitro PARG inhibition, cell permeability, a structure-activity-relationship, and protection in cell culture models established, the protective properties of PIP-1 are evaluated in cultures of primary dopaminergic neurons taken from the midbrain of embryonic rats. After isolation, these neurons are treated with 100 nM rotenone and a range of PIP-1 concentrations. Cells are stained with antibodies to MAP2 and tyrosine hydroxylase (TH) to determine the proportion of dopaminergic neurons remaining. Again, PIP-1 showed considerable protection in this assay (FIG. 20C). Importantly, compound 5 of FIG. 19, a compound that is structurally similar to PIP-1 but does not inhibit PARG, showed no protection in this assay (FIG. 20D).

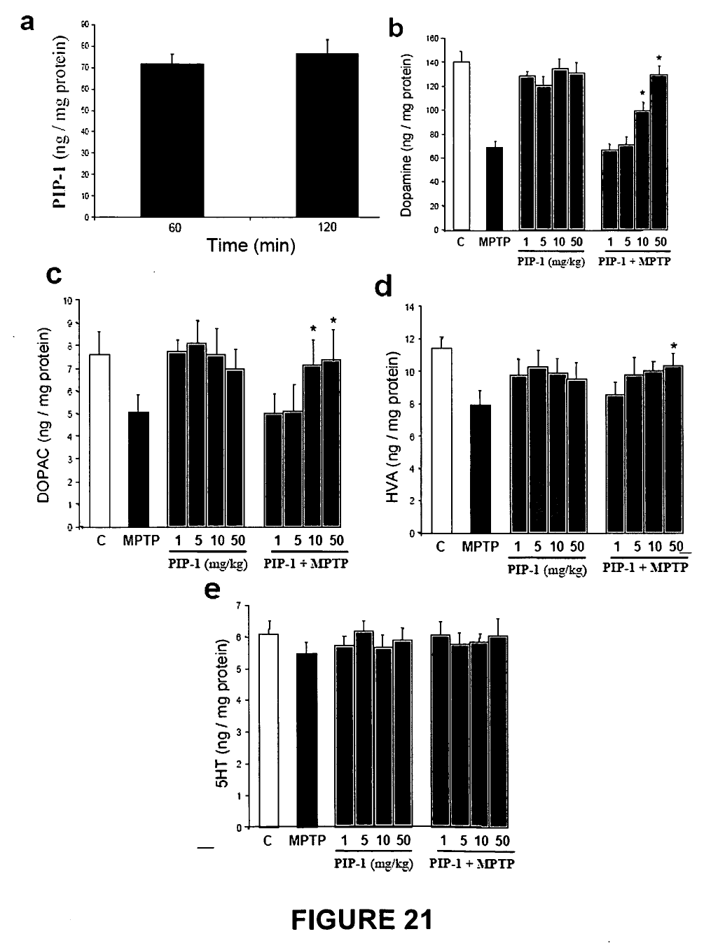

As a prelude to assessment of PIP-1 in a mouse model of PD, the ability of PIP-1 to penetrate the blood-brain-barrier is first assessed. PIP-1 is injected i.p. at a dose of 50 mg/kg, and mice are sacrificed at 1 and 2 h post injection. After sacrifice, to establish the presence of PIP-1 in the brain the striatum was dissected, sonicated in perchloric acid, and centrifuged. The clarified supernatant is analyzed by HPLC. No endogenous compounds interfere with this assay for PIP-1 as no signal is detected in dissected striata from non-treated mice. As shown by FIG. 21A, the striatal penetrance of PIP-1 is at the level of 70 ng/mg of protein. This amount of PIP-1 cannot be explained by compound in brain vessels in the striatum; for example, samples of peripheral blood at the same time points contain no detectable PIP-1. Given this blood-brain-barrier penetrance by PIP-1, the compound is assessed in the MPTP mouse model of PD. Mice are given a one-time i.p. injection of PIP-1 followed 30 minutes later by an injection of MPTP (30 mg/kg). PIP-1 is assessed at concentrations of 0 mg/kg, 1 mg/kg, 5 mg/kg, 10 mg/kg, and 50 mg/kg, and five mice are used in each group. After 30 days, the mice are sacrificed and the striatum dissected and analyzed by HPLC to determine the levels of dopamine and various dopamine metabolites. As shown in FIG. 21B-21D, MPTP-treated mice that received 50 mg/kg PIP-1 had levels of dopamine and dopamine metabolites very similar to the control mice that received no MPTP. In addition, PIP-1 by itself does not appear to have any adverse effects on the levels of dopamine or dopamine metabolites. As expected, neither MPTP nor PIP-1 has an effect on serotonin levels (FIG. 21E). Control experiments show no difference in MPP⁺ levels in the straita of PIP-1-treated and non-treated mice, thus PIP-1 is not simply altering the metabolism of MPP⁺.

None of the currently approved drugs for the treatment of PD offer neuroprotection or halt disease progression. Described herein is the first evidence suggesting that inhibition of PARG with a small molecule is an attractive strategy for preventing neurodegeneration in PD. The identification of PIP-1 as a non-toxic, potent, and cell permeable PARG inhibitor is critical to this process. Furthermore, based on common mechanisms of oxidative stress, small molecule inhibition of PARG is a viable treatment strategy for a variety of neurodegenerative diseases, and other disorders (such as stroke and myocardial infarction) that are due to oxidative stress and over-activation of PARP-1.

1. C. G. Goetz, W. Poewe, O. Rascol, C. Sampaio, Mov Disord 20, 523-39 (May, 2005). 2. A. J. Lees, R. Katzenschlager, J. Head, Y. Ben-Shlomo, Neurology 57, 1687-94 (Nov. 13, 2001). 3. C. E. Clarke, Lancet Neurol 3, 466-74 (August, 2004). 4. C. E. Clarke, Mov Disord 19, 491-8 (May, 2004). 5. W. Poewe, Neurology 66, S2-9 (May 23, 2006). 6. P. M. Abou-Sleiman, M. M. Muqit, N. W. Wood, Nat Rev Neurosci 7, 207-19 (March 2006). 7. S. Shimohama, H. Sawada, Y. Kitamura, T. Taniguchi, Trends Mol. Med. 9, 360-365 (2003). 8. J. W. Langston et al., Ann. Neuro. 46, 598-605 (1999). 9. J. T. Greenamyre, T. G. Hastings, Science 304, 1120-1122 (2004). 10. H. Wang, M. Shimoji, S. W. Yu, T. M. Dawson, V. L. Dawson, Ann. N.Y. Acad. Sci. 991, 132-139 (June 2003). 11. L. Davidovic, M. Vodenicharov, E. B. Affar, G. G. Poirier, Exp. Cell Res. 268, 7-13 (2001). 12. K. S. Putt, P. J. Hergenrother, Anal. Biochem. 333, 256-264 (2004). 13. D. W. Koh, T. M. Dawson, V. L. Dawson, Pharmacol Res 52, 5-14 (July, 2005). 14. J. Soos, J. I. Engelhardt, L. Siklos, L. Havas, K. Majtenyi, Neuroreport 15, 1715-8 (Aug. 6, 2004). 15. A. S. Mandir et al., Proc. Natl. Acad. Sci. 96, 5774-5779 (1999). 16. A. Iwashita et al., J Pharmacol Exp Ther 309, 1067-78 (June 2004). 17. C. Cosi, M. Marien, Ann N Y Acad Sci 890, 227-39 (1999). 18. C. Cosi, F. Colpaert, W. Koek, A. Degryse, M. Marien, Brain Res 729, 264-9 (Aug. 12, 1996). 19. S. Smith, Trends Biochem. Sci. 26, 174-179 (2001). 20. T. B. Sherer et al., J Neurosci 23, 10756-64 (Nov. 26, 2003). 21. G. J. Southan, C. Szabo, Curr. Med. Chem. 10, 321-340 (2003). 22. C. Szabo, V. L. Dawson, Trends Pharm. Sci. 19, 287-298 (1998). 23. J. Falsig et al., Eur. J. Pharmacol. 497, 7-16 (2004). 24. W. H. Ying, R. A. Swanson, Neuroreport 11, 1385-1388 (2000). 25. Y.-J. Tsai et al., J. Biol. Chem. 267, 14436-14442 (1992). 26. M. Tavassoli, T. Hajihosaini, S. Shall, Biochim. Biophysica. Acta 827, 228-234 (1985). 27. J. T. Slama et al., J. Med. Chem. 38, 389-393 (1995). 28. P. J. Hergenrother, Curr Opin Chem Biol 10, 213-8 (June 2006). 29. K. S. Putt, P. J. Hergenrother, Anal. Biochem. 326, 78-86 (2004). 30. K. S. Putt, G. J. Beilman, P. J. Hergenrother, Chembiochem 6, 53-55 (2005). 31. R. Betarbet et al., Nat Neurosci 3, 1301-6 (December, 2000). 32. C. Perier, J. Bove, M. Vila, S. Przedborski, Trends Neurosci 26, 345-6 (July, 2003).

Methods and Compound Manufacture

Cell culture conditions: HL-60 and SK-N-SH cells are grown in RPMI 1640 media supplemented with 10% FBS and PC-12 cells are grown in RPMI 1640 media supplemented with 10% HS and 5% FBS. All cell lines are incubated at 37° C. in a 5% CO₂, 95% air atmosphere. HL-60 cells are split every two to three days as needed and SK-N-SH cells are split when they reach approximately 90% confluency at a 1:6 ratio. SK-N-SH cells are differentiated by adding media containing 10 μM retinoic acid (in DMSO). Media is replaced every other day and cells are differentiated for a total of 12 days. PC-12 cells are differentiated by adding media containing 25 ng/mL Neuronal Growth Factro β. Media is replaced every other day and cells are differentiated for a total of 10 days. Differentiation is observed by microscopy.

Library screen: 12.5 μL of Lysing PARP buffer (50 mM Tris, 10 mM MgCl₂, pH 8.0, 1% Triton X-100) containing 100 μM NAD⁺ is added to each well of a 384-well plate. Library compounds at 10 mM in DMSO are transferred using a 384-well pin transfer device that delivers 0.1 μL. HL-60 cells are spun down at 200 g for 5 min. and resuspended in Lysing PARP buffer to a concentration of 8×10⁶ cells/mL. 10 μL of a 30% H₂O₂ solution is added per mL of cells. 12.5 μL of the activated cell lysate is added to the plates. The plates are incubated for 60 min. at 37° C. The concentration of NAD⁺ is then determined using a fluorescent assay¹. 10 μL of a 2 M KOH solution and 10 μL of a 20% acetophenone solution (in ethanol) is added to each well. The plate is then incubated at 4° C. for 10 min. 45 μL of a 88% formic acid solution was added to each well. The plate was then incubated for 5 min. in a 110° C. oven. Once the plate cools, the fluorescent intensity of each well is determined on a Criterion Analyst AD (Molecular Devices) with an excitation of 360 nm and an emission of 445 nm.

PARP assay: To determine the IC₅₀ values of the library hits, 20 μL of a 1 μM solution of NAD⁺ in PARP assay buffer (50 mM Tris, 10 mM MgCl2, pH 8.0), 10 μL of activated DNA (Trevigen, Gaithersburg Md.) at a concentration of 50 μg/mL (in PARP assay buffer), and 10 μL of the compounds at varying concentrations (in PARP assay buffer) is added into the wells of a 96-well plate. The reaction is initiated by adding 10 μL of PARP-1 (Trevigen, Gaithersburg Md.) at a concentration of 10 μg/mL (in PARP assay buffer), bringing the final concentration to 2 μg/mL PARP-1, 10 μg/mL DNA, and 400 nM NAD⁺ with varying concentrations of compounds in a total volume of 50 μL. The plate is incubated for 15 min at room temperature and the concentration of NAD⁺ is determined by the fluorescence method as described above.

PARG assay: Production and purification of PAR. PARP-1 (20 units) is added to 900 μL of PARP assay buffer containing 1 mM NAD⁺ and 22.5 μg activated DNA. This reaction was run at 37° C. for 30 min and stopped by the addition of 300 μL of cold 100% trichloroacetic acid. The proteins thus precipitated are collected by centrifugation at 18,000 g for 30 min at 4° C. After removal of the supernatant, 1 mL of 1 M NaOH and 100 mM EDTA are added to the pellet and incubated for 2 h at 37° C. to cleave the PAR from the PARP protein acceptor. Then 9 mL of PAR buffer A (250 mM ammonium acetate, 6 M guanidinium HCl, 10 mM EDTA, pH 9.0) is added to the PAR and applied to a PROSEP-PB (Millipore) phenyl boronate column that is pre-equilibrated with PAR buffer A. The flowthrough is then reapplied to the column to ensure that all PAR is bound. The column is then washed with 25 mL of PAR buffer A, followed by 10 mL of PAR buffer B (1 M ammonium acetate and 1 mM EDTA, pH 9.0). The bound PAR is eluted with 4 mL of water collected in 1-mL fractions. Fractions containing PAR are pooled and concentrated. The concentration, measured as monomer, is determined by measuring the absorbance at 258 nM. PAR has an extinction coefficient of 13,500 cm⁻¹ at 258 nm.

Inhibition of PARG Activity: To determine the PARG IC₅₀ values of the compounds, 39 μL of a 25.6 μM solution of PAR in PARG assay (50 mM KCl, 50 mM KH2PO4, pH 7.2) buffer and 1 μL of the compounds at varying concentrations (in PARG assay buffer) are added into the wells of a 384-well plate. The reaction is initiated by the addition of 10 μL of PARG (Biomol) at a concentration of 1 ng/ml (in PARG assay buffer), bringing the final concentration to 200 μg/ml PARG and 20 μM PAR with varying concentrations of compounds in a total volume of 50 μL. The plate is incubated for 20 min at room temperature. The amount of free ADP-ribose hemiacetals is determined by the addition of 5 μL of an aqueous 8.5M KOH solution and 5 μL of an aqueous 850 μM benzamidine solution to each well. The plate is then incubated for 10 min in an oven set at 110° C. The plate is allowed to cool and then read on a Criterion Analyst AD with an excitation of 360 nm and an emission of 445 nm.

Intracellular PAR staining: 1 mL of 10⁶ cells/mL PC-12 cells is added to the wells of a 24-well plate. Varying concentrations of PIP-1 or DMSO as a control are added to the cells and incubated for 2 h at 37° C. H₂O₂ is added to the cells to approximately 500 mM. After a 2 h incubation at 37° C., the cells are thoroughly washed in PBS and fixed overnight in 4° C. ethanol. The cells are again washed in PBS and stained with a 1:50 dilution of anti-poly(ADP-ribose) antibody for 4 h at 4° C. The cells are then washed in PBS and stained with a Cy-5 labeled secondary antibody for 4 h at 4° C. After washing with PBS, the fluorescent intensity of each cell is determined by flow cytometry.

Protection of Rotenone and MPP⁺ induced toxicity in PC-12 and SK-N-SH cells: SK-N-SH and PC-12 cells are neuronally differentiated as described above. 10 μM rotenone, 10 μM MPP⁺ and various concentrations of PIP-1 or its derivatives are added to the cells and incubated for 72 h. The cells are then washed in PBS and the viability is determined by adding propidium iodide to 1 μg/mL and measuring the fluorescent intensity of each cell by flow cytometry.

Preparation of Primary Mesencephalic Cultures: Primary Midbrain Cultures are prepared using a modified version of a previously described protocol². All of the procedures involving animal handling are reviewed and approved by the Purdue Animal Care and Use Committee. Briefly, whole brains are dissected from day 17 rat embryos, and the mesencephalic region containing the substantia nigra and ventral tegmental area are isolated stereoscopically. The mesencephalic neurons and glia are dissociated from neuronal tissue with trypsin (final concentration, 26 μg/mL in 0.9% [w/v] NaCl) and plated on coverslips previously treated with poly-L-lysine (5 μg/mL). The media consisted of DMEM, 10% (v/v) FBS, 10% (v/v) HS, penicillin (100 U/ml), and streptomycin (100 μg/ml). Four days later, the cells are treated for 48 h with AraC (20 μM) to inhibit the growth of glial cells.

Treatment of primary cultures with rotenone and PIP-1: AraC-treated primary cultures are incubated in fresh media for 72 h. The cells are then treated with fresh media supplemented with vehicle (DMSO, 0.1% [v/v]) or rotenone (100 nM), in the absence or presence of PIP-1 (1, 10, or 50 μM) or the control compound 5 (10 or 50 μM). After 48 h, the cells are analyzed immunocytochemically as described below.

Immunocytochemistry: Primary cells are fixed in 4% (w/v) paraformaldehyde in PBS for 30 min. The cells are permeabilized and blocked simultaneously for 1 h with PBS containing 1% (w/v) BSA, 10% (v/v) FBS, and 0.3% (v/v) Triton X-100. After washing with PBS, the cells are treated overnight at 4° C. with an anti-MAP2 monoclonal IgG (1:500) and an anti-TH polyclonal antibody (1:500) to monitor relative dopaminergic cell viability. After washing, the cells are treated for 1 h at room temperature with goat anti-mouse IgG conjugated to AlexaFluor 488 (1:1000) and goat anti-rabbit IgG conjugated to AlexaFluor 594 (1:2000). The primary and secondary antibodies are prepared by diluting stock antibody solutions in PBS with 1% (w/v) BSA. The coverslips are mounted onto slides using ProLong Gold Antifade reagent, dried at room temperature overnight, and sealed with clear nail polish. MAP2- and TH-immunoreactive primary neurons are counted in 10 randomly chosen observation fields for each experimental condition using a Nikon TE2000-U inverted fluorescence microscope at a total magnification of 200×. The data are expressed as the percentage of MAP2-positive neurons that are also TH-positive. Each experiment is repeated 3 to 6 times using embryonic neurons isolated from independent pregnant rats. Statistical analyses are carried out using the program GraphPad Prism, Version 4.0 (http://www.graphpad.com/prism/Prism.htm).

Assessment of PIP-1 levels in the brain: PIP-1 was injected into mice i.p. at 50 mg/kg and were sacrificed at 1 and 2 h post injection. To determine the amount of PIP-1 in the brain, the striatum was dissected, sonicated in 0.1 N perchloric acid and centrifuged. The clarified supernatant was then injected into an HPLC and the concentration of PIP-1 was determined by comparison to a standard curve. No endogenous compound interfered with the assay of PIP-1 as no signal was detected when striata from non-treated mice was injected.

Chromatographic conditions: A Beckman 125 dual pump equipped with a UV detector (166, Beckman) set at 250 nm, with an ODS column (250×4.6 mm, 5 um, Beckman) and a Rheodyne 7725i injector with a 50 μL loop is used for the analysis of PIP. The mobile phase consisted of methanol/water (45/55) filtered on a GH Polipro (Hydrophilic Polypropylene membrane, 47 mm, 0.2 um) filter before use. PIP-1 separation is performed isocratically at room temperature at a flow rate of 1 mL/min; the pump pressure is 1.01 Kpsi. The HPLC analysis is completed in 12 min, the retention time for PIP-1 is 7.56 min, with a limit of quantitation of 0.10 nmol/mL. The linear correlation between peak areas and concentrations is assessed in the range 0.30-200 nmol/ml with a correlation coefficient of 0.998.

Mouse PIP-1 and MPTP Treatments: C57 Black mice of 9 weeks of age (Charles River, Italy) are divided in the following groups: Controls (n=6) receiving saline; animals received a single i.p. injection of MPTP hydrochloride (36 mg/kg corresponding to 30 mg/kg of free MPTP; n=6); animals treated with various doses of PIP-1, 1 mg/kg (n=5), 5 mg/kg (n=5), 10 mg/kg (n=5) or 50 mg/kg (n=5); mice receiving one dose of MPTP (30 mg/kg, i.p.) after treatment with various doses of PIP-1 (n=8 for each group). Mice are sacrificed by decapitation after 30 days after the injection of saline or MPTP. The striata are dissected and used for the determination of monoamine levels.

Monoamine assay: The corpus striatum is homogenized by sonication in 0.6 ml of ice-cold 0.1 M PCA. Fifty μL of the homogenate are used for protein determination. The remaining aliquot is centrifuged at 8000 g for 10 min, and 20 μl of the supernatant was injected into an HPLC equipped with an autosampler 507 (Beckman Instruments, Fullerton, Calif.), a programmable solvent module 126 (Beckman), an analytical C-18 reverse-phase column kept at 30° C. (Ultrasphere ODS 5 μm, 80 Å pore, 250×4.6 mm (Beckman), and a Coulochem II electrochemical detector (ESA, Inc., Chelmsford, Mass.). The holding potentials are set at +350 and −350 mV for the detection of dopamine (DA), 3,5-dihydroxyphenylacetic acid (DOPAC), homovanillic acid (HVA), 5-hydroxytryptamine (5-HT) and 5-hydroxyindoleacetic acid (5-HIAA). The mobile phase consisted of 80 mM sodium phosphate, 40 mM citric acid, 0.4 mM EDTA, 3 mM 1-heptansulphonic acid and 8.5% methanol, brought to pH 2.75 with phosphoric acid (run under isocratic conditions, at 1 ml/min).

CHEMICAL METHODS: ¹H NMR and ¹³C NMR spectra were recorded on Varian unity 400 and on Varian unity 500 spectrometers in CDCl₃, CD₃OD, DMSO-d₆. The data is reported as follows: chemical shifts in ppm (δ), multiplicities are indicated as s-singlet; d-doublet; t-triplet; q-quarted; m-multiplet; br-broad. Coupling constants J are reported in Hz. Infrared spectra were recorded on Perkin Elmer spectrum BX spectrophotometer, and the peaks reported in cm⁻¹. Mass spectra data reported in m/e (intensity to 100%). Analytical thin-layer chromatography was performed on Merck silica gel plated with F254 indicator. Melting points were determined on a Thomas-Hoover Capillary Melting point Apparatus and are uncorrected. The starting material 4,5-Dichloro-2-phenyl-3(2H) pyridazinone; 4,5-Dichloro-2-methyl-3(2H) pyridazinone were prepared by reactions of mucochloric acid with the respective hydrazine in dilute hydrochloric acid.

The synthesis of PIP-1, and compounds 5 and 6 are shown in FIG. 22A.

Synthesis of 4-Methoxylbenzylamino-5-chloro-3(2H)pyridazinones (step i in FIG. 22A): A 10 mL vial is charged with the appropriate 4,5-dichloro-3(2H)pyridazinones (10 mmol), 4-methoxylbenzylamine (2.1 g, 15 mmol), Pd(OAc)₂ (1% mol), BINAP (2% mol), and potassium tert-butoxide (15 mmol). Anhydrous toluene (5 mL) is added into the mixture, and the vial is then sealed with a cap. The reaction mixture is stirred at 140° C. under microwave irradiation (300 W, 250 PSI) for 5 minutes. After the starting 4,5-dichloro-3(2H)pyridazinones is consumed, as monitored by TLC, it is found that two compounds are formed which are separated by eluting with hexane/ethyl acetate, the first fraction is product.

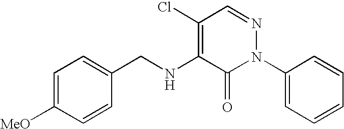

5-Chloro-4-(4-methoxy-benzylamino)-2-phenyl-2H-pyridazin-3-one Yield 76%, white solid. ¹H NMR (500 MHz, CDCl₃) δ ppm: 7.64 (s, 1H), 7.56 (d, 2H), 7.46 (t, 2H), 7.39 (t, 1H), 7.24 (d, 2H), 6.90 (d, 2H), 6.23 (br, 1H), 4.91 (d, 2H), 3.80 (s, 3H). ¹³C NMR (125 MHz, CDCl3) δ ppm: 159.4, 156.4, 141.6, 140.4, 140.3, 130.7, 129.0, 128.9, 128.5, 125.6, 114.4, 106.6, 55.5, 47.7. HRMS (ESI): m/e calcd for C₁₈H₁₆ClN₃O₂ (M+H⁺) 342.1009, found 342.1019. IR (KBr): 3325, 1610 cm⁻¹. m. p.=125-126° C.

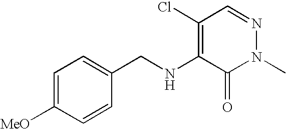

5-Chloro-4-(4-methoxy-benzylamino)-2-methyl-2H-pyridazin-3-one Yield 75%, white solid. ¹H NMR (400 MHz, CDCl3) δ ppm: 7.47 (s, 1H), 7.22 (d, 2H), 6.87 (t, 2H), 6.09 (br, 1H), 4.85 (d, 2H), 3.79 (s, 3H), 3.71 (s, 3H). ¹³C NMR (100 MHz, CDCl3) δ ppm: 159.3, 156.6, 139.7, 139.6, 130.8, 128.9, 114.4, 107.1, 55.5, 47.6, 40.2. HRMS (ESI): m/e calcd for C₁₃H₁₄ClN₃O₂ (M+H⁺) 280.0879, found 280.0870. IR (KBr): 3325, 1610 cm⁻¹. m. p.=94-96° C.

Synthesis of 4-Amino-5-chloro-3(2H)pyridazinones (step ii in FIG. 22A): In a 100 mL round flask, the appropriate 4-methoxylbenzylamino-5-chloro-3(2H)pyridazinone (100 mmol) was suspension in 10% HCl (50 mL), the mixture was heated at 100° C. for 6 h, the mixture was cooled down, extracted with CH₂Cl₂ (3×10 mL), the organic layer was combined together, dried with MgSO₄, the product was purified by silica gel with hexane/ethyl acetate (3:1) as elute to give product.

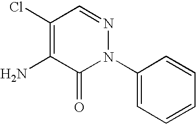

4-Amino-5-chloro-2-phenyl-2H-pyridazin-3-one, compound 5 Yield 92%, white solid. ¹H NMR (500 MHz, CDCl₃) δ ppm: 7.78 (s, 1H), 7.58 (d, 2H), 7.48 (t, 2H), 7.40 (t, 1H), 5.30 (br, 2H). ¹³C NMR (125 MHz, CDCl₃) δ ppm: 155.2, 141.4, 141.2, 138.4, 129.1, 128.6, 125.6, 109.2. HRMS (ESI): m/e calcd for C₁₀H₈ClN₃O (M+H⁺) 222.0518, found 222.0534. IR (KBr): 3330, 1610 cm⁻¹. m. p.=136-137° C.

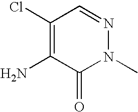

4-Amino-5-chloro-2-methyl-2H-pyridazin-3-one Yield 90%, white solid. ¹H NMR (500 MHz, CDCl₃) δ ppm: 7.58 (s, 1H), 5.24 (br, 2H), 3.75 (s, 3H). ¹³C NMR (125 MHz, CDCl₃) δ ppm: 155.6, 140.5, 137.6, 109.6, 40.0. HRMS (ESI): m/e calcd for C₅H₆ClN₃O (M+H⁺) 160.0225, found 160.0228. IR (KBr): 3330, 1608 cm⁻¹. m. p.=106-107° C.

Synthesis of 4-Acetylamino-5-chloro-3(2H)pyridazinones (step iii in FIG. 22A): To acetyl chloride (50 mL) in a round flask, the appropriate 4-amino-5-chloro-3(2H)pyridazinone (100 mmol) was added and the reaction mixture was stirred at 40° C. for 3 h. After the reaction mixture was cooled down, acetyl chloride was evaporated, and the residue was purified by silica gel using hexane/ethyl acetate (1:1).

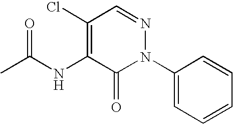

N-(5-Chloro-3-oxo-2-phenyl-2,3-dihydro-pyridazin-4-yl)-acetamide Yield 89%, white solid. ¹H NMR (500 MHz, CDCl₃) δ ppm: 8.31 (s, 1H), 7.91 (s, 1H), 7.53 (d, 2H), 7.49 (t, 2H), 7.43 (t, 1H), 2.13 (s, 3H). ¹³C NMR (125 MHz, CDCl₃) δ ppm: 167.5, 157.4, 140.9, 139.1, 134.0, 129.2, 129.0, 128.5, 125.5, 23.9. HRMS (ESI): m/e calcd for C₁₂H₁₀ClN₃O₂ (M+H⁺) 264.0540, found 264.0552. IR (KBr): 3330, 1610 cm⁻¹. m. p.=177-178° C.

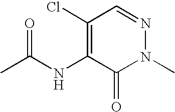

N-(5-Chloro-2-methyl-3-oxo-2,3-dihydro-pyridazin-4-yl)-acetamide Yield 90%, white solid. ¹H NMR (400 MHz, CDCl₃) δ ppm: 8.35 (s, 1H), 7.76 (s, 1H), 3.77 (s, 3H), 2.23 (s, 3H). ¹³C NMR (125 MHz, CDCl₃) δ ppm: 167.4, 157.9, 138.3, 133.0, 129.1, 40.6, 24.0. HRMS (ESI): m/e calcd for C₇H₈ClN₃O₂ (M+H⁺) 202.0358, found 202.0360. IR (KBr): 3325, 1610 cm⁻¹. m. p.=147-148° C.

Synthesis of 4-Acetylamino-5-(dimethyl amino)-3(2H)pyridazinones (step iv in FIG. 22A): A 10 mL vial was charged with the appropriate 4-acetylamino-5-chloro-3(2H)pyridazinone (10 mmol) and dimethylamine (15 mmol). Anhydrous THF (5 mL) was added into the mixture, and the vial was then sealed with a cap. The reaction mixture was stirred at 140° C. under microwave irradiation (300 W, 250 PSI) for 5 minutes. The solvent was evaporated and the compound was purified by silica gel using CH₂Cl₂/MeOH (20:1).

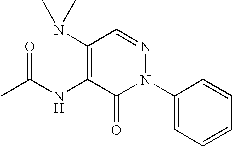

N-(5-Dimethylamino-3-oxo-2-phenyl-2,3-dihydro-pyridazin-4-yl)-acetamide Yield 96%, white solid. ¹H NMR (500 MHz, CDCl₃) δ ppm: 8.09 (s, 1H), 7.80 (s, 1H), 7.52 (d, 2H), 7.42 (t, 2H), 7.34 (t, 1H), 3.01 (s, 6H), 2.13 (s, 3H). ¹³C NMR (125 MHz, CDCl₃) δ ppm: 164.9, 159.5, 142.9, 141.6, 132.9, 128.8, 125.5, 112.5, 39.9, 23.2. HRMS (ESI): m/e calcd for C₁₄H₁₆N₄O₂ (M+H⁺) 273.1364, found 273.1356. IR (KBr): 3330, 1605 cm⁻¹. m. p.=186-187° C.

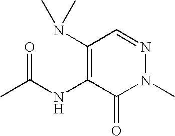

N-(5-Dimethylamino-2-methyl-3-oxo-2,3-dihydro-pyridazin-4-yl)-acetamide Yield 95%, white solid. ¹H NMR (500 MHz, CDCl₃) δ ppm: 8.45 (s, 1H), 7.66 (s, 1H), 3.68 (s, 3H), 3.00 (s, 6H), 2.19 (s, 3H). ¹³C NMR (125 MHz, CDCl₃) δ ppm: 164.7, 160.0, 144.2, 132.1, 112.2, 62.8, 40.2, 23.2. HRMS (ESI): m/e calcd for C₉H₁₄N₄O₂ (M+H⁺) 211.2358, found 211.2365. IR (KBr): 3320, 1605 cm⁻¹. m. p.=123-124° C.

Synthesis of 4-Amino-5-(dimethyl amino)-3(2H)pyridazinones (step v in FIG. 22A): To a solution of the appropriate 4-acetylamino-5-(dimethyl amino)-3(2H)pyridazinone (10 mmol) in ethanol, sodium ethanolate (5 mmol) was added and the reaction mixture was refluxed for 6 h. After the starting material was consumed, ethanol was evaporated, CH₂Cl₂ (20 mL) was added, washed with H₂O (3×10 mL), and the product was purified by silica gel using hexane/ethyl acetate (3:1).

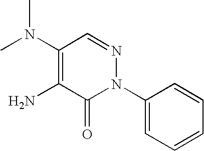

4-Amino-5-dimethylamino-2-phenyl-2H-pyridazin-3-one, PIP-1 Yield 96%, white solid. ¹H NMR (500 MHz, CDCl₃) δ ppm: 7.76 (s, 1H), 7.59 (d, 2H), 7.45 (t, 2H), 7.36 (t, 1H), 4.84 (br, 2H), 2.75 (s, 6H). ¹³C NMR (125 MHz, CDCl₃) δ ppm: 157.0, 142.1, 135.4, 133.7, 130.0, 128.9, 128.1, 125.7, 41.8. HRMS (ESI): m/e calcd for C₁₂H₁₄N₄O (M+H⁺) 273.1364, found 273.1356. IR (KBr): 3326, 1610 cm⁻¹. m. p.=188-189° C.

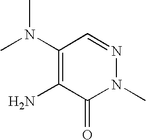

4-Amino-5-dimethylamino-2-methyl-2H-pyridazin-3-one, compound 6 Yield 94%, white solid. ¹H NMR (500 MHz, CDCl₃) δ ppm: 7.59 (s, 1H), 4.75 (s, 2H), 3.75 (s, 3H), 2.68 (s, 6H). ¹³C NMR (125 MHz, CDCl₃) δ ppm: 157.2, 134.9, 132.8, 130.5, 41.9, 39.9. HRMS (ESI): m/e calcd for C₇H₁₄N₄O (M+H⁺) 169.1254, found 169.1326 IR (KBr): 3325, 1610 cm⁻¹. m. p.=160-162° C.

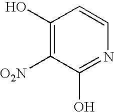

The synthesis of 4-9dimethylamino)-3-amino-2-phenylpyridinon (compound 4) is shown in FIG. 22B.

3-Nitro-pyridine-2,4-diol. A solution of 2,4-dihydroxypyridine (5.0 g, 45.0 mmol) in nitric acid (20 mL) was heated at 90° C. for 3 h. After cooling to room temperature, the solution was poured into crushed ice (50 mL). The resulting precipitate was filtered, washed with cold water, and dried to afford 6.46 g (92%) of the title compound as colorless solid.

¹H NMR (500 MHz, DMSO-d₃) δ ppm: 12.42 (brs, 1H), 11.87 (s, 1H), 7.42 (d, J=7.0, 1H), 6.00 (d, J=7.2, 1H). ¹³C NMR (125 MHz, DMSO-d₃) δ ppm: 161.3, 156.9, 138.8, 128.2, 98.7. HRMS (ESI): m/e calcd for C₅H₄N₂O₄ (M+H⁺) 157.0249, found 157.0256. m. p.>230° C.

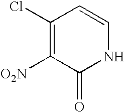

4-chloro-3-nitro-1H-pyridin-2-one. A solution of 3-nitro-pyridine-2,4-diol (5.0 g, 32.0 mmol) and cycloheptyl amine (4.0 g, 35.3 mmol) in MeOH (20 mL) was heated at 60° C. for 3 h. The solvent was evaporated, and the residue was taken up in POCl₃ (10 mL). The resulting solution was stirred at room temperature for 24 h. The volume of the solution was reduced by 70% in vacuo, and the cooled mixture was poured onto crushed ice. The resulting precipitate was filtered, washed with cold water, and dried to constant weight in vacuo to afford 5.1 g (91%) of the title compound as pale yellow solid.

¹H NMR (400 MHz, DMSO-d₆) δ ppm: 13.08 (brs, 1H), 7.76 (d, J=7.0, 1H), 6.58 (d, J=7.2, 1H). ¹³C NMR (100 MHz, DMSO-d₆) δ ppm: 154.9, 141.2, 139.7, 139.4, 106.5. HRMS (ESI): m/e calcd for C₅H₃ClN₂O₃ (M+H⁺) 174.9825, found 174.9913. m. p.=213-215° C.

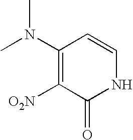

4-Dimethylamino-3-nitro-1H-pyridin-2-one. A 10 mL vial was charged with 4-chloro-3-nitro-1H-pyridin-2-one (2.0 g, 11.5 mmol) and dimethylamine (15 mmol). Anhydrous THF (5 mL) was added into the mixture, and the vial was then sealed with a cap. The reaction mixture was stirred at 140° C. under microwave irradiation (300 W, 250 PSI) for 5 minutes. The solvent was evaporated and purified by silica gel, the products were isolated by eluting with CH₂Cl₂/MeOH (20:1) to give 1.95 g (94%) of the product as yellow solid.

¹H NMR (500 MHz, DMSO-d₆) δ ppm: 11.31 (br s, 1H), 7.25 (d, J=7.0, 1H), 6.01 (d, J=7.2, 1H), 2.85 (d, 6H). ¹³C NMR (125 MHz, DMSO-d₆) δ ppm: 157.3, 152.4, 136.3, 124.6, 97.1, 41.4. HRMS (ESI): m/e calcd for C₇H₉N₃O₃ (M+H⁺) 184.0678, found 184.0698. m. p.=208-209° C.

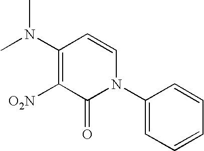

4-Dimethylamino-3-nitro-1-phenyl-1H-pyridin-2-one. A mixture of the 4-dimethylamino-3-nitro-1H-pyridin-2-one (1.5 g, 8.2 mmol), phenylboronic acid (1.5 g, 12.2 mmol), anhydrous cupric acetate (2.3 g, 12.2 mmol), activated 4 Å molecular sieves (1.5 g) pyridine (1.22 mL, 15.2 mmol) and triethylamine (2.1 mL, 15.2 mmol) in dichloromethane (30 mL) was treated at room temperature for 24 h. The reaction mixture was filtered through Celite, washed with dichloromethane and purified by flash chromatography on silica gel with hexane/ethylacetate (10:1) to provide 1.87 g (88%) of the product as yellow solid.

¹H NMR (400 MHz, CDCl₃) δ ppm: 7.43 (t, 2H), 7.37 (d, J=7.0, 1H), 7.31 (d, J=7.2, 2H), 7.23 (t, 1H), 6.00 (d, J=7.0, 1H), 3.04 (s, 6H). ¹³C NMR (100 MHz, CDCl₃) δ ppm: 156.7, 151.5, 139.9, 138.0, 129.4, 128.7, 126.7, 97.5, 41.3. HRMS (ESI): m/e calcd for C₁₃H₁₃N₃O₃ (M+H⁺) 260.1095, found 260.1087. m.p.=192-193° C.

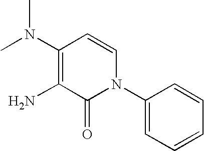

3-Amino-4-dimethylamino-1-phenyl-1H-pyridin-2-one, compound 4. 4-Dimethylamino-3-nitro-1-phenyl-1H-pyridin-2-one (1.0 g, 3.86 mmol) was dissolved in dioxane/water/NH₄OH (1:1:1, 10 mL). A solution of Na₂S₂O₄ (2.01 g, 11.58 mmol) in water (5 mL) was added. The reaction mixture was allowed to stir for 4 h at room temperature, then the mixture was filtered through Celite, washed with dichloromethane, and purified by flash chromatography on silica gel with hexane/ethylacetate (2:1) to provide 0.75 g (85%) of the product as white solid.

¹H NMR (400 MHz, CDCl₃) δ ppm: 7.45 (t, 2H), 7.38 (m, 3H), 6.81 (d, J=7.2, 1H), 6.17 (d, J=7.0, 1H), 4.10 (br s, 2H), 2.75 (s, 6H). ¹³C NMR (100 MHz, CDCl₃) δ ppm: 158.1, 141.4, 140.1, 129.3, 128.2, 128.0, 126.8, 125.6, 101.2, 41.5. HRMS (ESI): m/e calcd for C₁₃H₁₅N₃O (M+H⁺) 230.1268, found 230.1275. m. p.=128-129° C. ¹ Putt K S, Hergenrother P J. “An enzymatic assay for poly(ADP-ribose) polymerase-1 (PARP-1 via the chemical quantitation of NAD⁺: application to the high-throughput screening of small molecules as potential inhibitors.” Anal Biochem. 2004. 326:78-86.2 Zhou, W., Hurlbert, M. S., Schaack, J., Prasad, K. N., and Freed, C. R. (2000) Overexpression of human α-synuclein causes dopamine neuron death in rat primary culture and immortalized mesencephalon-derived cells. Brain Res 2000. 866:33-43.

The following describes information relevant to pharmaceutical and pharmacological embodiments and is further supplemented by information in the art available to one of ordinary skill. The exact formulation, route of administration and dosage can be chosen by an individual physician in view of a patient's condition (see e.g. Fingl et. al., in The Pharmacological Basis of Therapeutics, 1975, Ch. 1 p. 1).

It should be noted that the attending physician would know how to and when to terminate, interrupt, or adjust administration due to toxicity, or to organ dysfunctions, etc. Conversely, the attending physician would also know to adjust treatment to higher levels if the clinical response were not adequate (in light of or precluding toxicity aspects). The magnitude of an administered dose in the management of the disorder of interest can vary with the severity of the condition to be treated and to the route of administration. The severity of the condition may, for example, be evaluated, in part, by standard prognostic evaluation methods. Further, the dose and perhaps dose frequency, can also vary according to circumstances, e.g. the age, body weight, and response of the individual patient. A program comparable to that discussed above also may be used in veterinary medicine.

Depending on the specific conditions being treated and the targeting method selected, such agents may be formulated and administered systemically or locally. Techniques for formulation and administration may be found in Alfonso and Gennaro (1995) and elsewhere in the art. Suitable routes may include, for example, oral, rectal, transdermal, vaginal, transmucosal, or intestinal administration; parenteral delivery, including intramuscular, subcutaneous, or intramedullary injections, as well as intraocular, intrathecal, intravenous, or intraperitoneal administration.

For injection or other routes, agents of the invention may be formulated in aqueous solutions, preferably in physiologically compatible buffers such as Hanks' solution, Ringer's solution, water for injection, physiological saline buffer, or other solution. For transmucosal administration, penetrants appropriate to the barrier to be permeated can be used in the formulation. Such penetrants are generally known in the art.

Use of pharmaceutically acceptable carriers to formulate the compounds herein disclosed for the practice of the invention into dosages suitable for systemic or other administration is within the scope of the invention. With proper choice of carrier and suitable manufacturing practice, the compositions of the present invention, in particular those formulated as solutions, may be administered parenterally, such as by intravenous injection, or other routes. Appropriate compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, elixirs, solutions, suspensions and the like, e.g. for oral ingestion by a patient to be treated. For other routes, formulations can be prepared for creams, ointments, lotions, and the like.

Agents intended to be administered intracellularly may be administered using techniques well known to those of ordinary skill in the art. For example, such agents may be encapsulated into liposomes, other membrane translocation facilitating moieties, or other targeting moieties; then administered as described above. Liposomes can include spherical lipid bilayers with aqueous interiors. All molecules present in an aqueous solution at the time of liposome formation can be incorporated into the aqueous interior. The liposomal contents are both protected from the external microenvironment and, because liposomes fuse with cell membranes, are efficiently delivered into the cell cytoplasm. Additionally, due to hydrophobicity attributes, small organic molecules may be directly administered intracellularly.

Pharmaceutical compositions suitable for use in the present invention include compositions wherein the active ingredients are contained in an effective amount to achieve the intended purpose. Determination of the effective amounts is well within the capability of those skilled in the art, especially in light of the disclosure provided herein and other information in the art.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions, including those formulated for delayed release or only to be released when the pharmaceutical reaches the small or large intestine.

The pharmaceutical compositions of the present invention may be manufactured in a manner that is itself known, e.g., by means of conventional mixing, dissolving, suspending, granulating, dragee-making, levitating, emulsifying, encapsulating, entrapping, lyophilizing, and other processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are optionally provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

All references throughout this application, for example patent documents including issued or granted patents or equivalents; patent application publications; unpublished patent applications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference). In general the terms and phrases used herein have their art-recognized meaning, which can be found by reference to standard texts, journal references and contexts known to those skilled in the art.

When a group of substituents is disclosed herein, it is understood that all individual members of that group and all subgroups, including any isomers and enantiomers of the group members, are disclosed separately. When a Markush group or other grouping is used herein, all individual members of the group and all combinations and subcombinations possible of the group are intended to be individually included in the disclosure. It is intended that any one or more members of any Markush group or listing provided in the specification can be excluded from the invention if desired. When a compound is described herein such that a particular isomer or enantiomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Specific names of compounds are intended to be exemplary, as it is known that one of ordinary skill in the art can name the same compounds differently.

Molecules disclosed herein may contain one or more ionizable groups [groups from which a proton can be removed (e.g., —OH, —COOH, etc.) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. For example, in general any anions can be employed in the formation of salts of compounds herein; e.g. halide, sulfate, carboxylate, acetate, phosphate, nitrate, trifluoroacetate, glycolate, pyruvate, oxalate, malate, succinate, fumarate, tartarate, citrate, benzoate, methanesulfonate, ethanesulfonate, p-toluenesulfonate, salicylate and others.

Compounds of the present invention, and salts or esters thereof, may exist in their tautomeric form, in which hydrogen atoms are transposed to other parts of the molecules and the chemical bonds between the atoms of the molecules are consequently rearranged. It should be understood that all tautomeric forms, insofar as they may exist, are included within the invention. Additionally, the compounds may have trans and cis isomers and may contain one or more chiral centers, therefore existing in enantiomeric and diastereomeric forms. The invention can encompass all such isomers, individual enantiomers, as well as mixtures of cis and trans isomers, mixtures of diastereomers; non-racemic and racemic mixtures of enantiomers (optical isomers); and the foregoing mixtures enriched for one or more forms; except as stated otherwise herein. When no specific mention is made of the configuration (cis, trans or R or S) of a compound (or of an asymmetric carbon), then any one of the isomers or a mixture of more than one isomer is intended. The processes for preparation can use racemates, enantiomers, or diastereomers as starting materials. When enantiomeric or diastereomeric products are prepared, they can be separated by conventional methods, for example, by chromatographic or fractional crystallization. The inventive compounds may be in the free or hydrate form.

Every formulation or combination of components described or exemplified herein can be used to practice the invention, unless otherwise stated. Whenever a range is described in the present application, for example, a temperature range, a time range, or a composition or concentration range, all intermediate ranges and subranges, as well as all individual values included in the ranges given are intended to be included in the disclosure.

Information in any references disclosed herein can in some cases indicate the state of the art, for example for patent documents as of their effective filing dates; it is intended that such information can be employed herein, if needed, to exclude specific embodiments that are actually found to be in the prior art. For example, when a compound is disclosed and/or claimed, it should be understood that compounds qualifying as prior art with regard to the present invention, including compounds for which an enabling disclosure is provided in the references, are not intended to be included in the composition of matter claims herein.

Some references provided herein are incorporated by reference to provide details concerning sources of starting materials, additional starting materials, additional reagents, additional methods of synthesis, additional methods of analysis, assay methods, and additional uses of the invention. One of ordinary skill in the art will appreciate that starting materials, reagents, solid substrates, synthetic methods, purification methods, and analytical methods other than those specifically exemplified can be employed in the practice of the invention based on knowledge in the art and without resort to undue experimentation.

Where the terms “comprise”, “comprises”, “comprised”, or “comprising” are used herein, they are to be interpreted as specifying the presence of the stated features, integers, steps, or components referred to, but not to preclude the presence or addition of one or more other feature, integer, step, component, or group thereof. Separate embodiments of the invention are also intended to be encompassed wherein the terms “comprising” or “comprise(s)” or “comprised” are optionally replaced with the terms, analogous in grammar, e.g.; “consisting/consist(s)” or “consisting essentially of/consist(s) essentially of” to thereby describe further embodiments that are not necessarily coextensive. For clarification, as used herein “comprising” is synonymous with “having,” “including,” “containing,” or “characterized by,” and is inclusive or open-ended and does not exclude additional, unrecited elements or method steps. As used herein, “consisting of” excludes any element, step, component, or ingredient not specified in the claim element. As used herein, “consisting essentially of” does not exclude materials or steps that do not materially affect the basic and novel characteristics of the claim (e.g., not affecting an active ingredient). In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein. It is recognized that regardless of the ultimate correctness of any mechanistic explanation or hypothesis believed or disclosed herein, an embodiment of the invention can nonetheless be operative and useful.

The invention has been described with reference to various specific and preferred embodiments and techniques. However, it should be understood that many variations and modifications may be made while remaining within the spirit and scope of the invention. It will be appreciated by one of ordinary skill in the art that compositions, methods, devices, device elements, materials, optional features, procedures and techniques other than those specifically described herein can be applied to the practice of the invention as broadly disclosed herein without resort to undue experimentation. All art-known functional equivalents of compositions, methods, devices, device elements, materials, procedures and techniques described herein; and portions thereof; are intended to be encompassed by this invention. This invention is not to be limited by the embodiments disclosed, including any shown in the drawings or exemplified in the specification, which are given by way of example or illustration and not of limitation. The scope of the invention shall be limited only by the claims.
1. Bürkle, A. (2001). PARP-1: a regulator of genomic stability linked with mammalian longevity. Chembiochem 2, 725-728.
2. Meyer-Ficca, M. L., Meyer, R. G., Coyle, D. L., Jacobson, E. L., & Jacobson, M. K. (2004). Human poly(ADP-ribose) glycohydrolase is expressed in alternative splice variants yielding isoforms that localize to different cell compartments. Exp Cell Res 297, 521-532.
3. Davidovic, L., Vodenicharov, M., Affar, E. B., & Poirier, G. G. (2001). Importance of poly(ADP-ribose) glycohydrolase in the control of poly(ADP-ribose) metabolism. Exp Cell Res 268, 7-13.
4. Scovassi, A. I., & Poirier, G. G. (1999). Poly(ADP-ribosylation) and apoptosis. Mol Cell Biochem 199, 125-137.
5. Lazenik, Y. A., Kaufmann, S. H., Desnoyers, S., Poirier, G. G., & Earnshaw, W. V. (1994). Cleavage of poly(ADP-ribose) polymerase by a proteinase with properties like ICE. Nature 371, 346-347.
6. Putt, K. S., Beilman, G. J., & Hergenrother, P. J. (2005). Direct quantitation of poly(ADP-ribose) polymerase (PARP) activity as a means to distinguish necrotic and apoptotic death in cell and tissue samples. Chembiochem 6, 53-55.
7. Pieper, A. A., Verma, A., Zhang, J., & Snyder, S. H. (1999). Poly (ADP-ribose) polymerase, nitric oxide and cell death. Trends Pharmacol Sci 20, 171-181.
8. Dantzer, F., de La Rubia, G., Menissier-De Murcia, J., Hostomsky, Z., de Murcia, G., & Schreiber, V. (2000). Base excision repair is impaired in mammalian cells lacking poly(ADP-ribose) polymerase-1. Biochemistry 39, 7559-7569.
9. Shall, S., & de Murcia, G. (2000). Poly(ADP-ribose) polymerase-1: What have we learned from the deficient mouse model? Mutat Res 460, 1-15.
10. Hassa, P. O., & Hottiger, M. O. (1999). A role of poly (ADP-ribose) polymerase in NF-□□ transcriptional activation. Biol Chem 380, 953-959.
11. Ullrich, O., Reinheckel, T., Sitte, N., Hass, R., Grune, T., & Davies, K. J. (1999). Poly-ADP ribose polymerase activates nuclear proteasome to degrade oxidatively damaged histones. Proc. Nat. Acad. Sci. USA 96, 6223-6228.
12. Smith, S., Giriat, I., Schmitt, A., & de Lange, T. (1998). Tankyrase, a poly(ADP-ribose) polymerase at human telomeres. Science 282, 1484-1487.
13. Chang, P., Jacobson, M. K., & Mitchison, T. J. (2004). Poly(ADP-ribose) is required for spindle assembly and structure. Nature 432, 645-649.
14. Koh, D. W., Lawler, A. M.; Poitras, M. F.; Sasaki, M.; Wattler, S.; Nehls, M. C.; Stöger, T.; Poirer, G. G.; Dawson, V. L.; Dawson, T. M. (2004). Failure to degrade poly(ADP-ribose) causes increased sensitivity to cytotoxicity and early embryonic lethality. Proc Nat Acad Sci USA 101, 17699-17704.
15. Berger, N. A. (1985). Poly(ADP-ribose) in the cellular response to DNA damage. Radiat Res 4-15.
16. Li, J.-H., Ferraris, D. V., Kletzly, P. W., Li, W., Wang, E. Y., Xing, A. D., Xu, W., & Zhang, J. (2001). Symmetrically disubstituted aromatic compounds and pharmaceutical compositions for inhibiting poly (ADP-ribose) glycohydrolase, and methods for their use. U.S. Pat. No. 6,635,786.
17. Ying, W. H., Sevigny, M. B., Chen, Y. M., & Swanson, R. A. (2001) Poly(ADP-ribose) glycohydrolase mediates oxidative and excitotoxic neuronal death. Proc Nat Acad Sci USA 98, 12227-12232.
18. Hanai, S., Kanai, M., Ohashi, S., Okamoto, K., Yamada, M., Takahashi, H., & Miwa, M. (2004). Loss of poly(ADP-ribose) glycohydrolase causes progressive neurodegeneration in Drosophila melanogaster. Proc Nat/Acad Sci USA 101, 82-86.
19. Virag, L., & Szabo, C. (2002). The therapeutic potential of poly(ADP-ribose) polymerase inhibitors. Pharmacol Rev 54, 375-429.
20. Wilson, G. L., Patton, N. J., McCord, J. M., Mullins, D. W., & Mossman, B. T. (1984). Mechanisms of streptozotocin- and alloxan-induced damage in rat B cells. Diabetologia 27, 587-591.
21. Cantoni, O., Sestili, P., Spadoni, G., Balsamini, C., Cucchiarini, L., & Cattabeni, F. (1987). Analogues of benzamide containing a sulfur atom as poly(ADP-ribose) transferase inhibitors. Biochem Int 15, 329-337.
22. Szabo, C., Virag, L., Cuzzocrea, S., Scott, G. S., Hake, P., O'Connor, M. P., Zingarelli, B., Salzman, A., & Kun, E. (1998). Protection against peroxynitrite-induced fibroblast injury and arthritis development by inhibition of poly(ADP-ribose) synthase. Proc Nat/Acad Sci USA 95, 3867-3872.
23. Pion, E., Bombarda, E., Stiegler, P., Ullmann, G. M., Mely, Y., de Murcia, G., & Gerard, D. (2003). Poly(ADP-ribose) polymerase-1 dimerizes at a 5′ recessed DNA end in vitro: a fluorescence study. Biochemistry 43, 12409-12417.
24. Smith, S. (2001). The world according to PARP. Trends Biochem. Sci. 26, 174-179.
25. Perkins, E., Sun, D., Nguyen, A., Tulac, S., Francesco, M., Tavana, H., Nguyen, H., Tugendreich, S., Barthmaier, P., Couto, J., Yeh, E., Thode, S.; Jarnagin, K., Jain, A., Morgans, D., & Melese, T. (2001). Novel inhibitors of poly(ADP-ribose) polymerase/PARP1 and PARP2 identified using a cell-based screen in yeast. Cancer Res 61, 4175-4183.
26. Tentori, L., Portarena, I., & Graziani, G. (2002). Potential clinical applications of poly(ADP-ribose) polymerase inhibitors. Pharmacol Res 45, 73-85.
27. Southan, G. J., & Szabo, C. (2003). Poly(ADP-ribose) polymerase inhibitors. Curr Med Chem 10, 321-340.
28. Tsutsumi, M., Masutani, M., Nozaki, T., Kusuoka, O., Tsujiuchi, T., Nakagama, H., Suzuki, H., Konishi, Y., & Sugimura, T. (2001). Increased susceptibility of poly(ADP-ribose) polymerase-1 knockout mice to nitrosamine carcinogenicity. Carcinogenesis 22, 1-3.
29. Lu, X. C. M., Massuda, E., Lin, Q., Li, W. X., Li, J. H., & Zhang, J. (2003). Post-treatment with a novel PARG inhibitor reduces infarct in cerebral ischemia in the rat. Brain Res 978, 99-103.
30. Milam, K. M., & Cleaver, J. E. (1984). Inhibitors of poly(adenosine diphosphate-ribose) synthesis: effect on other metabolic processes. Science 223, 589-591.
31. Hatakeyama, K., Nemoto, Y., Ueda, K., & Hayaishi, O. (1986). Purification and characterization of poly(ADP-ribose) glycohydrolase. Different modes of action on large and small poly(ADP-ribose). J Biol Chem 1986, 261, 14902-14911.
32. Rapizzi, E., Fossati, S., Moroni, F., & Chiarugi, A. (2004). Inhibition of poly(ADP-ribose) glycohydrolase by gallotannin selectively up-regulates expression of proinflammatory genes. Mol Pharm 2004, 66, 890-898.
33. Bonicalzi, M. E., Vodenicharov, M., Coulombe, M., Gagne, J. P., & Poirier, G. G. (2003). Alteration of poly(ADP-ribose) glycohydrolase nucleocytoplasmic shuttling characteristics upon cleavage by apoptotic proteases. Biol Cell 95, 635-644.
34. Lin, W. S., Ame, J. C., Aboulela, N., Jacobson, E. L., & Jacobson, M. K. (1997). Isolation and characterization of the cdna encoding bovine poly(adp-ribose) glycohydrolase. J Biol Chem 272, 11895-11901.
35. Shimokawa, T., Masutani, M., Nagasawa, S., Nozaki, T., Ikota, N., Aoki, Y., Nakagama, H., & Sugimura, T. (1999). Isolation and Cloning of rat poly(ADP-ribose) glycohydrolase: presence of a potential nuclear export signal conserved in mammalian orthologs. J Biochem 126, 748-755.
36. Affar, E. B., Germain, M., Winstall, E., Vodenicharov, M., Shah, R. G., Salvesen, G. S., & Poirier, G. G. (2001). Caspase-3-mediated processing of poly(ADP-ribose) glycohydrolase during apoptosis. J Biol Chem 276, 2935-2942.
37. Alvarez-Gonzalez, R., & Althaus, F. R. (1989). Poly(ADP-ribose) catabolism in mammalian cells exposed to DNA-damaging agents. Mutat Res 218, 67-74.
38. Brochu, G., Duchaine, C., Thibeault, L., Lagueux, J., Shah, G. M., & Poirier, G. G. (1994). Mode of action of poly(ADP-ribose) glycohydrolase. Biochim Biophys Acta—Gene Structure & Expression 1219, 342-350.
39. Burzio, L. O., Riquelme, P. T., Ohtsuka, E., & Koide, S. S. (1976). Evidence for two variants of poly(ADP-ribose) glycohydrolase in rat testis. Arch Biochem Biophys 173, 306-331.
40. Ramsinghani, S., Koh, D. W., Amé, J.-C., Strohm, M., Jacobson, M. K., & Slama, J. T. (1998). Syntheses of photoactive analogues of adenosine diphosphate (hydroxymethyl)pyrrolidinediol and photoaffinity labeling of poly(ADP-ribose) glycohydrolase. Biochemistry 37, 7801-7812.
41. Minaga, T., & Kun, E. (1983). Spectral analysis of the conformation of polyadenosine diphosphoribose. Evidence indicating secondary structure. J Biol Chem 258, 725-730.
42. Koh, D. W., Patel, C. N., Ramsinghani, S., Slama, J. T., Oliveira, M. A., & Jacobson, M. K. (2003). Identification of an inhibitor binding site of poly(ADP-ribose) glycohydrolase Biochemistry 42, 4855-4863.
43. Koh, D. W., Coyle, D. L., Mehta, N., Ramsinghani, S., Kim, H., Slama, J. T., & Jacobson, M. K. (2003). SAR analysis of adenosine diphosphate (hydroxymethyl)pyrrolidinediol inhibition of poly(ADP-ribose) glycohydrolase. J Med Chem 46, 4322-4332.
44. Panda, S., Poirer, G. G., & Kay, S. A. (2002). tej defines a role for poly(ADP-ribosyl)ation in establishing period length of the Arabidopsis circadian oscillator. Dev Cell 3, 51-61.
45. Menegazzi, M., Grassi-Zucconi, G., Carcerero De Prati, A., Ogura, T., Poltronieri, P., Nyunoya, H., Shiratori-Nyunoya, Y., Miwa, M., & Suzuki, H. (1991). Differential expression of poly(ADP-ribose) polymerase and DNA polymerase beta in rat tissues. Exp Cell Res 197, 66-94.
46. Cortes, U., Tong, W. M., Coyle, D. L., Meyer-Ficca, M. L., Meyer, R. G., Petrilli, V., Herceg, Z., Jacobson, E. L., Jacobson, M. K., & Wang, Z. Q. (2004). Depletion of the 110-kilodalton isoform of poly(ADP-ribose) glycohydrolase increases sensitivity to genotoxic and endotoxic stress in mice. Mol Cell Biol 24, 7163-7178.
47. Miwa, M., & Sugimura, T. (1971). Splitting of the ribose-ribose linkage of poly(adenosine diphosphate-ribose) by calf thymus extract. J Biol Chem 246, 6362-6364.
48. Brochu, G., Shah, G. M., & Poirier, G. G. (1994). Purification of poly(adp-ribose) glycohydrolase and detection of its isoforms by a zymogram following one- or two-dimensional electrophoresis. Anal Biochem 218, 265-272.
49. Thomassin, H., Jacobson, M. K., Guay, J.; Verreault, A., Aboul-ela, N., Menard, L., & Poirier, G. G. (1990). An affinity matrix for the purification of poly(ADP-ribose) glycohydrolase. Nucl Acids Res 18, 4691-4694.
50. Shah, G. M., Poirer, D., Duchaine, C., Brochu, G., Desnoyers, S., Lagueux, J., Verreault, A., Hoflack, J. C., Kirkland, J. B., & Poirer, G. G. (1995). Methods for biochemical study of poly(ADP-ribose) metabolism in vitro and in vivo. Anal Biochem 227, 1-13.
51. Winstall, E., Affar, E. B., Shah, R., Bourassa, S., Scovassi, A. I., & Poirier, G. G. (1999). Poly(ADP-ribose) glycohydrolase is present and active in mammalian cells as a 110-kDa protein. Exp Cell Res 246, 395-398.
52. Affar, E. B., Germain, M., Winstall, E., Vodenicharov, M., Shah, R. G., Salvesen, G. S., & Poirer, G. G. (2001). Caspase-3-mediated processing of poly(ADP-ribose) glycohydrolase during apoptosis. J Biol Chem 276, 2935-2942.
53. Maruta, H., Inageda, K., Aoki, T., Nishina, H., & Tanuma, S. (1991). Characterization of two forms of poly(ADP-ribose) glycohydrolase in guinea pig liver. Biochemistry 30, 5907-5912.
54. Ménard, L., & Poirier, G. G. (1987). Rapid assay of poly(ADP-ribose)glycohydrolase. Biochem Cell Biol 65, 668-673.
55. Pacheco-Rodriguez, G., & Alvarez-Gonzalez, R. (1999). Measurement of poly(ADP-ribose) glycohydrolase activity by high resolution polyacrylamide gel electrophoresis: Specific inhibition by histones and nuclear matrix proteins. Mol Cell Biochem 193, 13-18.
56. Masutani, M., Shimokawa, T., Igarashi, M., Hamada, M., Shibata, A., Oami, S., Nozaki, T., Nakagama, H., Sugimura, T., Takeuchi, T., & Hori, M. (2002). Inhibition of poly(ADP-ribose) glycohydrolase activity by cyclic peptide antibiotics containing piperazic acid residues. Proc Japan Acad Ser B, 78, 15-17.
57. Slama, J. T., Aboul-Ela, N., & Jacobson, M. K. (1995). Specific inhibition of poly(ADP-ribose) glycohydrolase by adenosine diphosphate hydroxymethyl)pyrrolidinediol. J Med Chem 38, 4332-4336.
58. Maruta, H., Matsumura, N., & Tanuma, S. (1997). Role of (ADP-ribose) catabolism in DNA repair. Biochem Biophys Res Comm 236, 265-269.
59. Putt, K. S., & Hergenrother, P. J. (2004). A nonradiometric, high-throughput assay for poly(ADP-ribose) glycohydrolase (PARG): application to inhibitor identification and evaluation. Anal Biochem 333, 256-264.
60. Kai, M., Tamura, K., Yamaguchi, M., & Ohkura, Y. (1985). Aromatic amidines as fluorogenic reagents for reducing carbohydrates. Anal Sci 1, 255-263.
61. Coquet, A., Veuthey, J. L., & Haerdi, W. (1991). Selective post-column fluorogenic reaction with benzamidine for trace level detection of reducing saccharides in liquid chromatography. J Chromatography 552, 255-263.
62. Kakita, H., Kamishima, H., Komiya, K., & Kato, Y. (2002). Simultaneous analysis of monosaccharides and oligosaccharides by high-performance liquid chromotography with post column fluorescence derivatization. J Chromatography 961, 77-82.
63. Tavassoli, M., Tavassoli, M. H., & Shall, S. (1985). Effect of DNA intercalators on poly(ADP-ribose) glycohydrolase activity. Biochim Biophys Acta 827, 228-234.
64. Bernardi, R., Rossi, L., Poirer, G. G., & Scovassi, A. I. (1997). Analysis of poly(ADP-ribose) glycohydrolase activity in nuclear extracts from mammalian cells. Biochim Biophys Acta 1338, 60-68.
65. Tsai, Y.-J., Aoki, T., Maruta, H., Abe, H., Sakagami, H., Hatano, H., Okuda, T.; & Tanurna, S. (1992). Mouse mammary tumor virus gene expression is suppressed by oligomeric ellagitannins, novel inhibitors of poly(ADP-ribose) glycohydrolase. J Biol Chem 267, 14436-14442.
66. Aoki, K., Nishimura, K., Abe, H., Maruta, H., Sakagarni, H., Hatano, T., Okuda, T., Yoshida, T., Tsai, Y.-J., Uchiumi, F., & Tanuma, S-I. (1993). Novel inhibitors of poly(ADP-ribose) glycohydrolase. Biochim Biophys Acta 1158, 251-256.
67. Keil, C., Petermann, E., & Oei, S. L. (2004). Tannins elevate the level of poly(ADP-ribose) in HeLa cell extracts. Arch Biochem Biophys 425, 115-121.
68. Genovese, T., Di Paola, R., Catalano, P., Li, J. H., Xu, W. Z., Massuda, E., Caputi, A. P., Zhang, J., & Cuzzocrea, S. (2004). Treatment with a novel poly (ADP-ribose) glycohydrolase inhibitor reduces development of septic shock-like syndrome induced by zymosan in mice. Crit. Care Med. 32, 1365-1374.
69. Hassa, P. O., & Hottiger, M. O. (2002). The functional role of poly(ADP-ribose)polymerase 1 as novel coactivator of NF-kappaB in inflammatory disorders. Cell Mol Life Sci 59, 1534-1553
70. Lefer, A. M., & Lefer, D. J. (1993). Pharmacology of the endothelium in ischemia-reperfusion and circulatory shock. Annu Rev Pharmacol Toxicol 33, 71-90.
71. Oei, S. L., & Ziegler, M. (2000). ATP for the DNA Ligation Step in Base Excision Repair Is Generated from Poly(ADP-ribose). J Biol Chem 275, 23234-23239.
72. Di Meglio, S., Tramontano, F., Cimmino, G., Jones, R., & Quesada, P. (2004). Dual role for poly(ADP-ribose)polymerase-1 and -2 and poly(ADP-ribose)glycohydrolase as DNA-repair and pro-apoptotic factors in rat germinal cells exposed to nitric oxide donors. Biochim. Biophys. Acta—Mol Cell Res 1692, 35-44.
73. Masson, M., Niedergang, V., Schreiber, S., Muller, J., Menissier-de Murcia, G., & de Murcia, G. (1998). XRCC1 is specifically associated with poly(ADP-ribsoe) polymerase and negatively regulates its activity following DNA damage. Mol Cell Biol 18, 3563-3571.
74. Ziegler, M., & Oei, S. L. (1999). Poly(ADP-ribosyl)ation stimulates DNA repair and silence transcription. BioEssays 23, 543-547.
75. Lautier, D., Laguexu, J., Thibodeau, J., Menard, L., & Poirier, G. G. (1993). Molecular and biochemical features of polyADP(ribose) metabolism. Mol Cell Biochem 122, 171-193.
76. D'Amours, D., Desnoyers, S., D'Silva, I., & Poirier, G. G. (1999). Biochem J 342, 249-268.
77. Zahradka, P., & Ebisuzaki, K. (1982). A shuttle mechanism for DNA-protein interactions. The regulation of poly(ADP-ribose) polymerase. Eur J Biochem 127, 579-585.
78. Ying, W. H.; & Swanson, R. A. (2000). The poly(ADP-ribose) glycohydrolase inhibitor gallotannin blocks oxidative astrocyte death. Neuroreport 11, 1385-1388.
79. Tanaka, Y., Yoshihara, K., Itaya, A., Kamiya, T., & Koide, S. S. (1984). Mechanism of the inhibition of Ca2+, Mg2+-dependent endonuclease of bull seminal plasma induced by ADP-ribosylation. J Biol Chem 259, 6579-6585.
80. Yakovlev, A. G., Wang, G., Stoica, B. A., Boulares, H. A., Spoonde, A. Y., Yoshihara, K., & Smulson, M. E. (2000). A Role of the Ca²⁺/Mg²⁺-dependent endonuclease in apoptosis and its inhibition by poly(ADP-ribose) polymerase. J Biol Chem 275, 21302-21308.
81. Dumitriu, I. E., Voll, R. E., Kolowos, W., Gaipl, U.S., Heyder, P., Kalden, J. R., & Herrmann, M. (2004). UV irradiation inhibits ABC transporters via generation of ADP-ribose by concerted action of poly(ADP-ribose) polymerase-1 and glycohydrolase. Cell Death Differ 11, 314-320.
82. Deliconstantinos, G., Villiotou, V., & Stavrides, J. C. (1996). Increase of particulate nitric oxide synthase activity and peroxynitrite synthesis in UVB-irradiated keratinocyte membranes. Biochem J 320, 997-1003.
83. Cole, S. P., Bhardwaj, G., Gerlach, J. H., Mackie, J. E., Grant, C. E., Almquist, K. C., Stewart, A. J., Kurz, E. U., Duncan, A. M., & Deeley, R. G. (1992). Overexpression of a transporter gene in a multi-drug resistant human lung cancer cell line. Science 258, 1650-1654.
84. de Murcia, G., Huletsky, A., LAmarre, D., Gaudreau, A., Poujet, J., Daune, M., & Poirier, G. G. (1986). Modulation of chromatin superstructure induced by poly(ADP-ribose) synthesis and degredation. Proc Natl Acad Sci USA 261, 7011-7017.
85. de Murcia, G., Huletsky, A., & Poirier, G. G. (1988). Modulation of chromatin structure by poly(ADP-ribosyl)ation. Biochem Cell Biol 66, 626-635.
86. Falsig, J., Christiansen, S. H., Feuerhahn, S., Bürkle, A., Oei, S. L., Keil, C., & Leist, M. (2004). Poly(ADP-ribose) glycohydrolase as a target for neuroprotective intervention: assessment of currently available pharmacological tools. Eur J of Pharmacol 497, 7-16.
87. Yokozawa, T., Chen, C. P., Dong, E., Tanaka, T., Nonaka, G. I., & Nishioka, I. (1998). Study on the inhibitory effect of tannins and flavonoids against the 1,1-diphenyl-2 picrylhydrazyl radical. Biochem Pharmacol 56, 213-222.
88. Bakondi, E., Bai, P., Szabó, E., Hunyadi, J., Gergely, P., Szabó, C., & Virag, L. (2002). Detection of poly(ADP-ribose) polymerase activation in oxidatively stressed cells and tissues using biotinylated NAD substrate. J Histochem Cytochem 50, 91-98.
89. Masutani, M., Nakagama, H., & Sugimura, T. (2003). Poly(ADP-ribose) and carcinogenesis. Genes, Chromosomes & Cancer 38, 339-348.
90. Tong, W.-M.; Cortes, U.; Wang, Z.-Q. “Poly(ADP-ribose) polymerase: a guardian angel protecting the genome and suppressing tumorigenesis” Biochim. Biophys. Acta 2001, 1552, 27-37.
91. Shimohama, S.; Sawada, H.; Kitamura, Y.; Taniguchi, T. “Disease model: Parkinson's disease” Trends Mol. Med. 2003, 9, 360-365.
92. Wang, H.; Shimoji, M.; Yu, S. W.; Dawson, T. M.; Dawson, V. L. “Apoptosis inducing factor and PARP-mediated injury in the MPTP mouse model of Parkinson's disease.” Ann. N.Y. Acad. Sci. 2003, 991, 132-199.
93. Yu, S.-W.; Wang, H.; Poitras, M. F.; Coombs, C.; Bowers, W. J.; Federoff, H. J.; Poirier, G. G.; Dawson, T. M.; Dawson, V. L. “Mediation of poly(ADP-ribose) polymerase-1-dependent cell death by apoptosis-inducing factor” Science 2002, 297, 259-263.
94. Mandir, A. S.; Przedborski, S.; Jackson-Lewis, V.; Wang, Z.-Q.; Simbulan-Rosenthal, C. M.; Smulson, M. E.; Hoffman, B. E.; Guastella, D. B.; Dawson, V. L. “Poly(ADP-ribose) polymerase activation mediates 1-methyl-4-phenyl-1,2,3,6-tetrahydropyridine (MPTP)-induced parkinsonism” Proc. Nat. Acad. Sci. 1999, 96, 5774-5779.
95. Cosi, C. “New inhibitors of poly(ADP-ribose) polymerase and their potential therapeutic targets” Expert Opin. Ther. Patents 2002, 12, 1047-1070.
96. Cosi, C., et al. “Poly(ADP-ribose) polymerase (PARP) revisited. A new role for an old enzyme: PARP involvement in neurodegeneration and PARP inhibitors as possible neuroprotective agents.” Ann. N.Y. Acad. Sci. 1997, 825, 366-379.
97. Cosi, C.; Colpaert, F.; Koek, W.; Degryse, A.; Marien, M. “Poly(ADP-ribose) polymerase inhibitors protect against MPTP-induced depletions of striatal dopamine and cortical noradrenaline in C57B1/6 mice.” Brain Res. 1996, 729, 264-269.
98. Cosi, C.; Marien, M. “Implication of poly (ADP-ribose) polymerase (PARP) in neurodegeneration and brain energy metabolism. Decreases in mouse brain NAD+ and ATP caused by MPTP are prevented by the PARP inhibitor benzamide” Ann. N.Y. Acad. Sci. 1999, 890, 227-239.
99. Cosi, C.; Marien, M. “Decreases in mouse brain NAD+ and ATP induced by 1-methyl-4-phenyl-1,2,3,6-tetrahydropyridine (MPTP): prevention by the poly(ADP-ribose) polymerase inhibitor, benzamide.” Brain Res. 1998, 809, 58-67.
100. Nottbohm, A. C.; Hergenrother, P. J.; chapter in Drug Discover Research in the Post Genomics Era (Huang, Z., Ed.), Wiley (submitted).
101. Kitamura Y, K. T., Kakimura J I, Matsuoka Y, Kohno Y, Nomura Y, Taniguchi T. “Protective Effects of the Antiparkinsonian Drugs Talipexole and Pramipexole against 1-Methyl-4-phenylpyridinium-Induced Apoptotic Death in Human Neuroblastoma SH-SY5Y Cells” Mol Pharmacol 1998, 54, 1046-1054

We claim:

1. A method of treating a neurodegenerative condition in a patient comprising administering a compound having a chemical structure FX1: 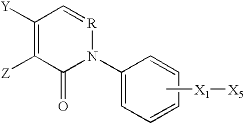
wherein R is N;
Y is NR₁R₂, of the structure: 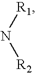
halide or acyl, wherein R₁ and R₂ are independently alkyl groups ranging from between one and about ten carbons inclusive, hydrogen, aryl, alkyl aryl, substituted alkyl, where one or more substituents are halides, or R₁ and R₂ can be alkyl groups where two alkyl groups form a cyclic moiety having 5, 6, 7 or 8 ring members where one ring carbon can be O;
Z is NR₄R₅, of the structure: 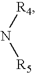
halide, or a group that is converted under physiological conditions to NH₂; wherein R₄ and R₅ independently can have values of R₁ and R₂, or 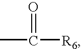
where R₆ is H or small alkyl; and X₁-X₅ independently, =hydrogen, halide, alkyl, alkylhalide, OH, OR₇, where R₇ is alkyl.

2. The method of claim 1, wherein the structure is FX4:

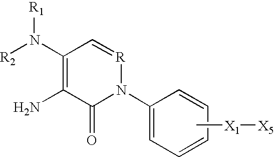

3. The method of claim 1, wherein the structure is PIP-1:

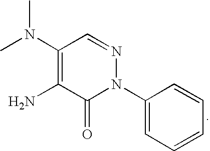

4. The method of claim 1, wherein the compound is selected from the group consisting of:

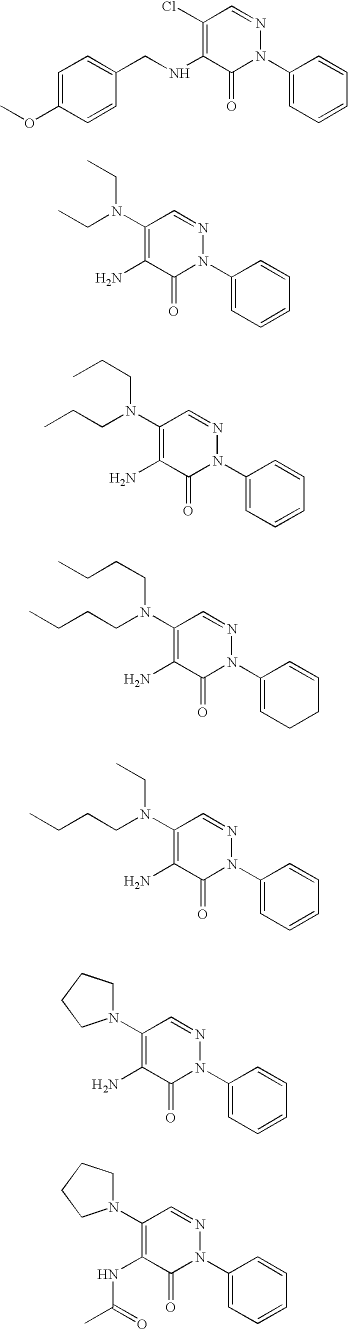

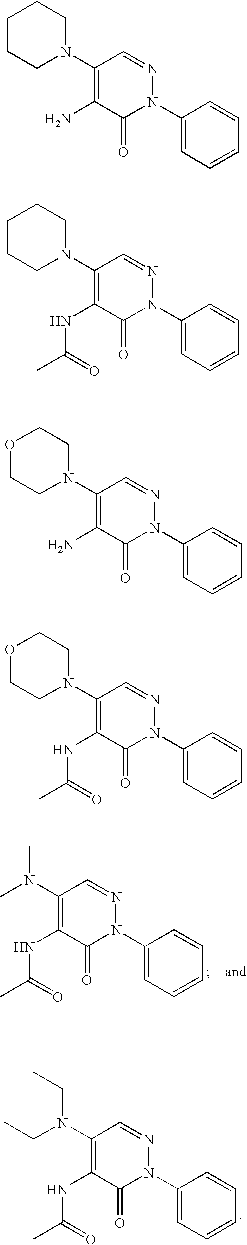

5. The method of claim 4, wherein the compound is selected from the group consisting of:

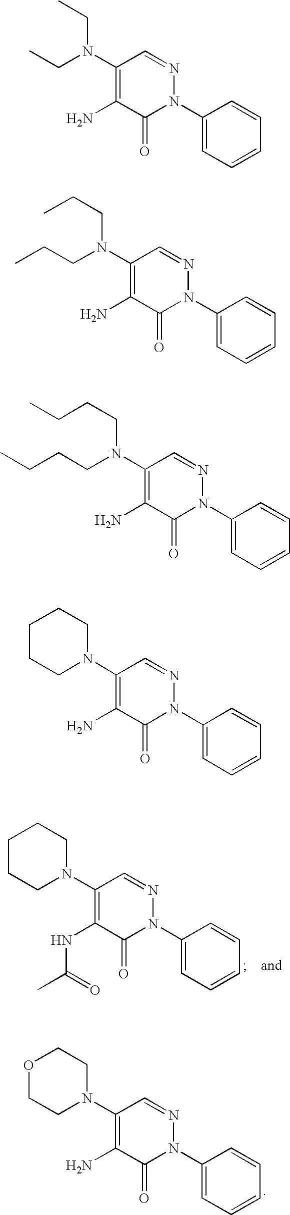

6. The method of claim 4, wherein the compound is selected from the group consisting of:

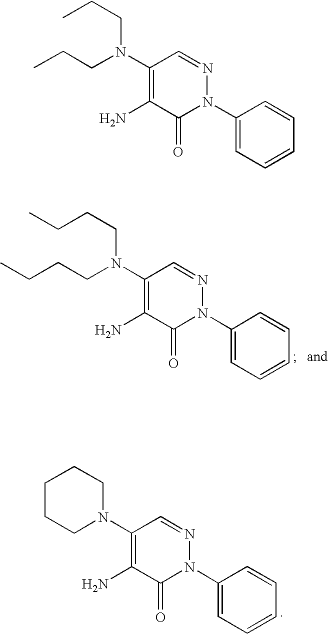

7. The method of claim 1, wherein said neurodegenerative condition is in a Parkinson's patient.

8. The method of claim 1, wherein said administered compound selectively inhibits PARG in a target cell in said patient, wherein said target cell is a neuronal cell or neurodegenerative cell.

9. The method of claim 8, wherein said compound does not substantially inhibit a PARP molecule in said target cell.

10. The method of claim 8, wherein said compound does not substantially inhibit a PARP-1 molecule in said target cell.

11. The method of claim 8, wherein said target cell is in a Parkinson's patient.

12. The method of claim 1, wherein said neurodegenerative condition is from an ischemic event.

13. A method of selectively inhibiting PARG in a target cell comprising administering to said target cell a compound having a chemical structure FX1: 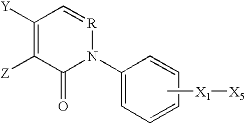
wherein R is N;
Y is NR₁R₂, of the structure: 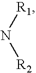
halide or acyl, wherein R₁ and R₂ are independently alkyl groups ranging from between one and about ten carbons inclusive, hydrogen, aryl, alkyl aryl, substituted alkyl, where one or more substituents are halides, or R₁ and R₂ can be alkyl groups where two alkyl groups form a cyclic moiety having 5, 6, 7 or 8 ring members where one ring carbon can be O;
Z is NR₄R₅, of the structure: 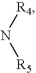
halide, or a group that is converted under physiological conditions to NH₂; wherein R₄ and R₅ independently can have values of R₁ and R₂, or 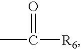
where R₆ is H or small alkyl; and X₁-X₅ independently, =hydrogen, halide, alkyl, alkylhalide, OH, OR₇, where R₇ is alkyl.

14. The method of claim 13, wherein said compound does not substantially inhibit a PARP molecule in said target cell.

15. The method of claim 13, wherein said target cell is a neuronal cell or a neurodegenerative cell.

16. The method of claim 15, wherein said target cell is in a Parkinson's patient.

17. The method of claim 15, wherein said target cell is an ischemic cell.

18. The method of claim 13, wherein the compound has structure:

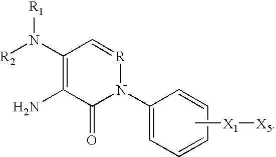

19. The method of claim 13, wherein the compound has structure: [chemical structure]
